(19) United States
(12) Patent Application Publication
(10) Pub. No.: US 2026/0232838 A1
(43) Pub. Date: Aug. 13, 2026

(54) SYNTHETIC MELANIN NANOPARTICLES FOR PROTECTION OF CELL-FREE REACTIONS

(71) Applicant: Northwestern University, Evanston, IL (US)

(72) Inventors: Nathan C. GIANNESCHI, Evanston, IL (US); Lauren M. IRIE, Evanston, IL (US); Dylan M. BROWN, Evanston, IL (US); Julius B. LUCKS, Evanston, IL (US)

(73) Assignee: Northwestern University, Evanston, IL (US)

(21) Appl. No.: 19/533,146

(22) Filed: Feb. 7, 2026

Related U.S. Application Data
(60) Provisional application No. 63/755,965, filed on Feb. 7, 2025.

(51) Int. Cl. A61K 47/69; A61P 39/06

(52) U.S. Cl. CPC A61K 47/6933; A61P 39/06

(57) ABSTRACT

Provided herein are synthetic melanin nanoparticles and methods useful for protecting cell-free reactions from prolonged solar and UV exposure.

### CROSS-REFERENCE TO RELATED APPLICATIONS

This application claims the benefit of and priority to U.S. Provisional Patent Application No. 63/755,965, filed Feb. 7, 2025, which is hereby incorporated by reference in its entirety.

### STATEMENT REGARDING FEDERALLY SPONSORED RESEARCH OR DEVELOPMENT

This invention was made with government support under W52P1J-21-9-3023 awarded by the Other Agency, and W911NF-22-2-0246 awarded by the Army Research Laboratory-Army Research Office. The government has certain rights in the invention.

### BACKGROUND OF THE INVENTION

Cell-free expression systems, such as E. coli-based cell-free expression systems, have been applied for point-of-use technologies such as antibody and peptide production as well as for rapid biosensors for environmental water quality monitoring. These systems are compatible with freeze drying, and previous studies have assessed additives for lyoprotection and long-term storage. Ultra violet (UV) light is a commonly known sterilization technique, however, it is widely known that UV exposure can have damaging biological effects. There is an opportunity to expand cell-free additives for the prevention of biological damage caused by UV exposure events, which has not yet been characterized for certain cell-free expression systems, such as E. coli cell-free expression systems.

Thus, there remains a need in the art for protection of cell-free reactions.

### SUMMARY OF THE INVENTION

Aspects disclosed herein include an additive, for example for protecting a cell-free reaction or cell-free reaction environment from damage, wherein the additive comprises a synthetic melanin nanoparticle, wherein the synthetic melanin nanoparticle comprises a synthetic melanin oligomer or a synthetic melanin polymer, or a combination of a synthetic melanin oligomer and a synthetic melanin polymer.

Also disclosed herein is a method for protecting a cell-free reaction from damage, the method comprising contacting a cell-free reaction, or an environment thereof, with an additive to generate a cell-free reaction mixture, wherein the additive comprises a synthetic melanin nanoparticle, wherein the synthetic melanin nanoparticle comprises a synthetic melanin oligomer or a synthetic melanin polymer, or a combination of a synthetic melanin oligomer and a synthetic melanin polymer.

Without wishing to be bound by any particular theory, there may be discussion herein of beliefs or understandings of underlying principles relating to the devices and methods disclosed herein. It is recognized that regardless of the ultimate correctness of any mechanistic explanation or hypothesis, an embodiment of the invention can nonetheless be operative and useful.

### BRIEF DESCRIPTION OF THE DRAWINGS

FIGS. 1A-1F: Characterization of example synthetic melanin nanoparticles. STEM SE micrograph images of AMNP-1 (FIG. 1A); AMNP-2 (FIG. 1B); PDA (FIG. 1C); DLS (FIG. 1D); UV-Vis absorption spectra (FIG. 1E); ABTS radical scavenging assay results (FIG. 1F). Scale bar for FIGS. 1A-1C=500 μm.

FIGS. 2A-2F: Example of effect of melanin derivative nanoparticles on cell-free gene expression. FIG. 2A: Melanin nanoparticles AMNP-1, AMNP-2, and PDA in 10 mg/mL solution. FIG. 2B: Schematic of cell-free sfGFP expression in the presence of increasing concentrations of melanin nanoparticles for highly processive T7 RNAP-based gene expression and native E. coli RNAP gene expression. FIG. 2C: pT7-sfGFP expression (MEF (μM FITC)) across multiple concentrations (0, 0.25, 0.5, 1, 2, and 3 mg/ml) of melanin nanoparticles. FIG. 2D: Representative images of the pT7-sfGFP cell-free reactions after 20 h at 30° C. before (pre-spin—rows 1 and 2) and after centrifugation (post-spin-rows 3 and 4) and in light (rows 1 and 3) or under UV imager (rows 2 and 4). FIG. 2E: pJ23119-sfGFP expression (MEF (UM FITC)) across multiple melanin nanoparticle concentrations (0, 0.25, 0.5, 1, 2, and 3 mg/mL). FIG. 2F: Representative images of the pJ23119-sfGFP cell-free reactions after 20 h at 30° C. before (pre-spin-rows 1 and 2) and after centrifugation (post-spin-rows 3 and 4) and in light (rows 1 and 3) or under UV imager (rows 2 and 4). For FIGS. 2C-2F, All reactions were made to 20 UL in polymerase chain reaction (PCR) tubes, contained 20 nM pT7-sfGFP or pJ23119-sfGFP DNA, and were incubated for 20 h at 30° C. Fluorescence data were calibrated to mean equivalent fluorescence (MEF (UM FITC)). For each of FIGS. 2D and 2F, each set of 6 tubes shows different melanin nanoparticles concentration conditions: 0, 0.25, 0.5, 1, 2, 3 mg/mL (left to right).

FIGS. 3A-3C: Melanin nanoparticles can protect cell-free reactions from otherwise deleterious light exposure events. FIG. 3A: Schematic of example experimental workflow for assessing protection against UVB or solar lamp exposure. FIG. 3B: Heatmaps for UVB and solar lamp protection from 2.1, and 0.5 mg/mL concentrations with pT7 sfGFP expression. Heatmaps show normalized expression to the no nanoparticle, no light control for a given nanoparticle condition. FIG. 3C: Heatmaps for UVB and solar lamp protection imparted by melanin nanoparticles at 2, 1, and 0.5 mg/mL concentrations for pJ23119 sfGFP expression. Heatmaps show average normalized expression for UVB and solar lamp protection over a range of exposure times (0, 15, 30, or 60 min) for nanoparticle concentrations of 2, 1, and 0.5 mg/mL with pT7-sfGFP expression (FIG. 3B) or pJ23119-sfGFP expression (FIG. 3C). Normalized expression was calculated as the expression of the exposed reactions divided by no light control (t=0 min) for each row (individual nanoparticle condition). Heatmaps of FIGS. 3B and 3C show average normalized expression (three replicates) with respect to the FITC-calibrated GFP expression in the no light exposure control (t=0 min) for each given nanoparticle condition. All reaction volumes were 20 μL in PCR tubes, contained 20 nM pT7-sfGFP or pJ23119-sfGFP DNA, and were incubated for 20 h at 30° C. post light exposure.

FIGS. 4A-4C: Tuning DNA concentration can improve reaction efficacy in conjunction with the protective effects of melanin nanoparticles for cell-free expression systems. FIG. 4A: Schematic showing the approach for UVB exposure over different DNA concentrations. FIG. 4B: pT7 expression of sf-GFP for 10 and 30 nM DNA template concentrations with AMNP-1, AMNP-2 and PDA particles over a range of UVB exposure times. FIG. 4C: pJ23119 expression of sfGFP for 10 and 30 nM DNA template concentrations with AMNP-1 and 2, and PDA particles over a range of UVB exposure times. Heatmaps show average normalized expression (three replicates) with respect to the FITC-calibrated GFP expression in the no light exposure control (t=0 min) for each given nanoparticle condition. Reaction volumes were all 20 μL in PCR tubes, contained 1 mg/mL nanoparticles, and were incubated for 20 h at 30° C. post light exposure.

FIGS. 5A-5C: Melanin nanoparticles can protect lyophilized reactions from strong UVB exposure. FIG. 5A: Schematic of experimental process to determine lyophilization compatibility and protection with melanin nanoparticle additives. FIG. 5B: Average normalized pT7 expression of sfGFP post-lyophilization and UVB or solar lamp exposure over a range of exposure times. FIG. 5C: Average normalized pJ23119-sfGFP expression post-lyophilization and UVB or solar lamp exposure. Heatmaps show average normalized expression (three replicates) with respect to the FITC-calibrated GFP expression in the no light exposure control (t=0 min) for each given nanoparticle condition. Reaction volumes were all 20 μL in PCR tubes, contained 0 or 1 mg/mL nanoparticles and 20 nM pT7-sfGFP or pJ23119-sfGFP DNA. Reactions were then lyophilized for 16 h, exposed to UVB or solar lamp light conditions on a cooling plate to 4° C., and then incubated for 20 h at 30° C. post light exposure.

FIGS. 6A-6B: Melanin nanoparticles can protect cell-free reactions from light exposure events. FITC calibrated expression values for UVB and solar lamp exposure experiments corresponding to FIGS. 3A-3C for pT7-sfGFP expression (FIG. 6A) and pJ23119-sfGFP (FIG. 6B). Heatmaps in FIG. 3B (pT7-sfGFP expression) and FIG. 3C (pJ23119-sfGFP expression) were calculated by using these values and normalizing to the FITC calibrated GFP expression at the t=0 min exposure time condition for each respective type of nanoparticle. All reaction volumes were 20 μL in PCR tubes, contained 20 nM pT7-sfGFP or pJ23119-sfGFP DNA, and were incubated for 20 h at 30° C. post light exposure. Fluorescence data calibrated to mean equivalent fluorescence (MEF).

FIGS. 7A-7B: Characterization of alternative radiation protectant additives. FIG. 7A: ABTS radical scavenging activity (%) for silica nanoparticles (SiNPs), zinc oxide (ZnO), L-ascorbic acid and resorcinol. FIG. 7B: UV-Vis absorption spectra (A.U.), for silica nanoparticles (SiNPs), zinc oxide (ZnO), L-ascorbic acid and resorcinol.

FIGS. 8A-8B: Characterization of metal oxide nanomaterials. SE-STEM micrographs of SiNPs (FIG. 8A) and ZnO nanopowder (FIG. 8B). Scale bar=500 nm for each of FIGS. 8A and 8B.

FIGS. 9A-9B: Inhibitory effects of alternative radical scavenging or light scattering materials on cell-free reactions. FIG. 9A: pJ23119-sfGFP expression of GFP in the presence of increasing concentrations of silica nanoparticles, resorcinol, zinc oxide nanopowder, or ascorbic acid. FIG. 9B: pT7-sfGFP expression in the presence of increasing concentrations (0, 0.25, 0.5, 1, 2, and 3 mg/mL) of the alternative protective additives. For pJ23119-sfGFP expression, higher concentrations demonstrated reduced expression activity. For pT7-sfGFP, other than ZnO nanopowder, the additives demonstrated a minimal inhibitory effect. In both cases, ZnO was observed to inhibit cell-free reactions at all concentrations tested. All reactions were made to a 20 μL volume in PCR tubes, contained 20 nM pT7-sfGFP or pJ23119-sfGFP DNA, and were incubated for 20 h at 30° C. Fluorescence data calibrated to mean equivalent fluorescence (MEF).

FIGS. 10A-10D: Alternative UV protectants do not shield cell-free reactions from the negative effects of UVB or Solar lamp exposure. FIG. 10A: Normalized reaction efficiency (%) post UVB and solar lamp exposure for pT7-sfGFP gene expression. FIG. 10B: Non-normalized FITC calibrated values for the data in FIG. 10A. FIG. 10C: Normalized reaction efficiency (%) post UVB and solar lamp exposure for pJ23119-sfGFP expression. FIG. 10D: Non-normalized FITC calibrated values for the data in FIG. 10C. Heatmaps in FIG. 10A and FIG. 10C are normalized to the FITC calibrated GFP expression at the t=0 min exposure, or the no UVB or solar lamp exposure condition, for each respective alternative radiation protectant. Reactions with alternative protectants are not protected against UVB or solar exposure in all conditions, showing that previously reported radical scavengers or light scattering particles are not functional for UV protection in cell-free reactions. All reactions were made to a 20 μL volume in PCR tubes, containing 20 nM pT7-sfGFP or pJ23119 DNA and were incubated for 20 h at 30° C. post light exposure. Fluorescence data calibrated to mean equivalent fluorescence (MEF).

FIGS. 11A-11B: FITC calibrated expression values for UVB exposure for gene expression using different DNA concentrations corresponding to FIGS. 4A-4C. FIG. 11A: pT7-sfGFP expression post exposure (corresponding to FIG. 4B) for 10 nM and 30 nM DNA concentrations. FIG. 11B: pJ23119-sfGFP expression post exposure (corresponding to FIG. 4C) for 10 nM and 30 nM DNA concentrations. Reaction volumes were all 20 UL in PCR tubes, containing 1 mg/mL nanoparticles and were incubating for 20 h at 30° C. post light exposure. Heatmaps in FIG. 4B (pT7-sfGFP expression) and FIG. 4C (pJ23119-sfGFP expression) were calculated by using these values and normalizing to the FITC calibrated GFP expression at the t=0 min exposure time condition for each respective type of nanoparticle. Fluorescence data calibrated to mean equivalent fluorescence (MEF).

FIGS. 12A-12B: FITC calibrated GFP expression from lyophilized reactions exposed to UVB or solar lamp exposure corresponding to FIGS. 5A-5C. FIG. 12A: pT7-sfGFP FITC calibrated expression values (corresponding to FIG. 5B). FIG. 12B: pJ23119-sfGFP FITC normalized expression values (corresponding to FIG. 5C). Reaction volumes were all 20 UL in PCR tubes, containing 0 or 1 mg/mL nanoparticles and 20 nM pT7-sfGFP or pJ23119-sfGFP DNA. Reactions were then lyophilized for 16 h, exposed to UVB or solar lamp light conditions on a cooling plate set to 4° C., and then incubated for 20 h at 30° C. post light exposure. Heatmaps in FIG. 5B (pT7-sfGFP expression) and FIG. 5C (pJ23119-sfGFP expression) were calculated by using these values and normalizing to the FITC calibrated GFP expression at the t=0 min exposure time condition for each respective type of nanoparticle. Fluorescence data calibrated to mean equivalent fluorescence (MEF).

FIG. 13: Additional schematic of example experimental workflow for assessing protection against UVB or solar lamp exposure.

FIGS. 14A-14C: Synthesis and characterization of NMNPs. FIG. 14A: SE-STEM micrographs of neo-melanin particles (NMNP) candidates. Scale bar=500 nm. Insets show optical image of NMNPs in water. FIG. 14B: UV-Vis spectra of NMNP candidates and p-1,8-DNH in water at 0.01 mg/mL. FIG. 14C: ABTS radical scavenging activity of NMNP candidates and p-1,8-DHN.

FIGS. 15A-15C: Compatibility of NMNPs with cell-free expression. FIG. 15A: Schematic of cell-free expression (CFE) reactions in the presence of 1 mg/mL NMNPs for pT7 RNAP and pJ23119 E. coli RNAP controlled sfGFP expression. FIG. 15B: Normalized fluorescence signal difference of NMNPs between nanoparticle removed overnight expression reactions and plate reader analyzed reactions to no nanoparticle (NP) controls. FIG. 15C: Overnight cell-free expression reaction endpoint fluorescence after nanoparticle removal for AMNP-1 and NMNPs 1 to 6 for pT7 and pJ23119 promoters. FIG. 15D: Images of NP containing reactions before incubation, after incubation, and after centrifugation under white light and a fluorescence visualizer device. FIG. 15E: Plate reader assay for reactions containing AMNP-1 and NMNPs 1 to 6 for pT7 and pJ23119 promoters. All reactions were made to 20 μL. Overnight reactions were incubated for 20 h at 30° C., spun down with centrifugation, then 5 μL were plated for analysis. 5 μL of bulk reactions were distributed for kinetic analysis on a plate reader and ran for 10 h at 30° C. with 5 minute read intervals.

FIGS. 16A-16I: Cell-free reactions with neo-allomelanins. FIG. 16A: Schematic of experimental workflow for assessing NMNP radiation protection of cell-free expression against UVB or solar lamp exposure with and without lyophilization. Heatmaps show normalized expression average over a range of exposure times (0, 15, 30 or 60 min) for No NP control, AMNP-1, NMNP-2 and NMNP-6, resorcinol, and ascorbic acid. FIG. 16B: pT7-sfGFP UVB exposure. FIG. 16C: pT7-sfGFP solar lamp exposure FIG. 16D: pJ23119-sfGFP UVB exposure. FIG. 16E: pJ23119-sfGFP solar lamp exposure. FIG. 16F: pT7-sfGFP UVB exposure post lyophilization. FIG. 16G: pT7-sfGFP solar lamp exposure post lyophilization. FIG. 16H: pJ23119-sfGFP UVB exposure post lyophilization. FIG. 16I: pJ23119-sfGFP solar lamp exposure post lyophilization. Normalized expression was calculated as the expression of the exposed reactions divided by no light control (t=0 min) for each row (individual nanoparticle condition). Heatmaps show average normalized expression (three replicates) with respect to the FITC calibrated GFP expression in the no light exposure control (t=0 min) for each given nanoparticle condition. All reaction volumes were 20 μL in PCR tubes, contained 20 nM pT7-sfGFP or pJ23119-sfGFP DNA, and were incubated for 20 h at 30° C. post light exposure.

FIGS. 17A-17C: Cell-free protection by neo-allomelanins. FIG. 17A: Schematic of an example experimental workflow for assessing NMNP radiation protection of tetR sensors. Inset of FIG. 17A provides a larger image of aTC. Heatmaps of FIGS. 17B and 17C show average normalized expression in the sensor “on” and “off” conditions over a range of exposure times (0, 15, 30 or 60 min) for NMNP-2, NMNP-5, and NMNP-6 under UVB (FIG. 17B) and solar lamp (FIG. 17C) exposure. Normalized expression was calculated as the expression of the exposed reactions divided by no light control (t=0 min) for each row (individual nanoparticle condition). Heatmaps show average normalized expression (three replicates) with respect to the FITC calibrated GFP expression in the no light exposure control (t=0 min) for each given nanoparticle condition. All reaction volumes were 20 μL in PCR tubes, contained 20 nM pT7-sfGFP or pJ23119-sfGFP DNA, and were incubated for 20 h at 30° C. post light exposure. 0 light condition over 0 light condition/normalized the on. Max fluorescence over t=20 h. Particles were centrifuged out in order to measure max fluorescence.

FIGS. 18A-18I: Polymerized DHN-monomers. UV-Vis absorption spectra and inset optical image of polymerized: 1,3-DHN (FIG. 18A); 1,4-DHN (FIG. 18B); 1,5-DHN (FIG. 18C); 1,6-DHN (FIG. 18D); 1,7-DHN (FIG. 18E); 1,8-DHN (FIG. 18F); 2,3-DHN (FIG. 18G); 2,6-DHN (FIG. 18H); and 2,7-DHN (FIG. 18I).

FIGS. 19A-19F: MALDI-MS spectra of NMNPs results for: NMNP-1 (FIG. 19A); NMNP-2 (FIG. 19B); NMNP-3 (FIG. 19C); NMNP-4 (FIG. 19D); NMNP-5 (FIG. 19E); and NMNP-6 (FIG. 19F).

FIG. 20: Thermogravimetric analysis (TGA) of NMNPs compared AMNP-1. TGA shows that AMNP-1 has much higher stability at higher temperatures compared to the NMNP library. This higher temperature stability implies a greater degree of intermolecular crosslinking and hence a larger pi-pi conjugated network, which gives AMNP-1 its characteristic dark, black pigment. In contrast, the lighter visible pigmentation of the NMNPs is likely due to its lesser degree of intermolecular crosslinking, which is reflected in its lower temperature stability compared to AMNP-1.

FIGS. 21A-21B: DLS and zeta potential measurements of candidate NMNPs. FIG. 21A: DLS and FIG. 21B: Zeta potential of NMNP-2, NMNP-5, and NMNP-6.

### STATEMENTS REGARDING CHEMICAL COMPOUNDS AND NOMENCLATURE

The term “amine compound” refers to a compound or molecule having one or more amine groups, where each amine group may be a primary, secondary, or tertiary amine group.

Wherein the term “alpha carbon” is used to describe an amine compound, the alpha carbon refers to a carbon closest to an N of an amine group. An alpha carbon that is a secondary carbon has one H directly bound to it (e.g., a secondary alpha carbon is bound to an N, two other carbons, and one H). An alpha carbon that is a tertiary carbon has no H directly bound to it (e.g., a tertiary alpha carbon is bound to an N and three other carbons).

The term “melanin” generally refers to one or more compounds or materials that function as a pigment, such as when internalized or taken up by a biological cell, for example. It is also noted that melanin is not necessarily taken up by cells. Melanin can be used for forming cell walls in fungi, for example, such as to provide rigidity, defense mechanisms, and more. In another illustrative example, melanin is used by birds, such as where melanin is organized in a matrix of keratin or similar type of biological material, where it can be organized into monolayers or multilayers to provide structural color, warmth, and more. A melanin compound or material may be, but is not limited to, a melanin monomer, a melanin oligomer, a melanin polymer, a melanin nanoparticle, a melanin layer (e.g., a melanin thin film or coating), or other melanin material, for example. For example, melanin nanoparticles internalized by a biological cell function as a pigment in the cell.

The terms “artificial melanin” and “synthetic melanin” are used interchangeably herein and refer to one or more melanin compounds, molecules, or materials, such as melanin monomers, melanin oligomers, or melanin nanoparticles, that are synthesized and are at least partially, or in some embodiments preferably entirely, not derived from or not extracted from a natural source, such as a biological source, a living organism, or a once living organism. The terms “synthetic” and “artificial” are used interchangeably herein when referring to a melanin or a material comprising a melanin. The terms “synthetic melanin nanoparticles” and “artificial melanin nanoparticles” are used interchangeably herein, and are intended to have the same meaning throughout the present disclosure, and refer to nanoparticles formed of artificial melanin, such as artificial melanin monomers and/or artificial melanin oligomers. The terms “synthetic melanin thin film” and “artificial melanin thin film” are used interchangeably herein, and are intended to have the same meaning throughout the present disclosure, and refer to a thin film formed of artificial melanin, such as artificial melanin monomers and/or artificial melanin oligomers. The terms “synthetic melanin layer” and “artificial melanin layer” are used interchangeably herein, and are intended to have the same meaning throughout the present disclosure, and refer to a layer formed of artificial melanin, such as artificial melanin monomers and/or artificial melanin oligomers. An artificial melanin nanoparticle, artificial melanin thin film, artificial melanin layer, and any compound, material, or formulation comprising any of these, comprises artificial melanin monomers, artificial melanin oligomers, and/or artificial melanin polymers. Optionally, an artificial melanin nanoparticle, artificial melanin thin film, artificial melanin layer, and any compound, material, or formulation comprising any of these, consists of or consists essentially of artificial melanin, such as artificial melanin monomers, artificial melanin oligomers, and/or artificial melanin polymers. Optionally, an artificial melanin nanoparticle, artificial melanin thin film, artificial melanin layer, and any compound, material, or formulation comprising any of these, is free (or substantially free) of artificial melanin monomers and comprises artificial melanin oligomers and/or artificial melanin polymers. Optionally, each artificial melanin monomer, artificial melanin oligomer, and artificial melanin polymer of an artificial melanin nanoparticle, artificial melanin thin film, artificial melanin layer, and any compound, material, or formulation comprising any of these, is not bound to, conjugated to, attached to, coated by, encompassed by or chemically otherwise associated with a natural or biological proteinaceous lipid. A natural or biological proteinaceous lipid refers to a naturally or biologically derived lipid or a lipid extracted from a natural or biological source, such as a once living organism, said lipid comprising one or more proteins such as the lipid (plasma) membrane of a melanocyte or melanosome). Optionally, each artificial melanin monomer, artificial melanin oligomer, and artificial melanin polymer of an artificial melanin nanoparticle, artificial melanin thin film, artificial melanin layer, and any compound, material, or formulation comprising any of these, is not bound to, conjugated to, attached to, coated by, encompassed by or otherwise chemically associated with a natural or biological lipid (e.g. a lipid bilayer, lipid membrane or phospholipid compound). A natural or biological lipid refers to a naturally or biologically derived lipid or a lipid extracted from a natural or biological source, such as a once living organism. Optionally, any artificial melanin monomer, artificial melanin oligomer, and artificial melanin polymer of an artificial melanin nanoparticle, artificial melanin thin film, artificial melanin layer, and any compound, material, or formulation comprising any of these, is bound to, conjugated to, attached to, coated by, encompassed by, and/or otherwise associated with a synthetic or artificial lipid or with a synthetic or artificial phospholipid. A synthetic or artificial lipid refers to a synthesized lipid that is not derived from or is not extracted from a natural or biological source, such as a once living organism.

The term “artificial melanin precursor” refers to a compound or material that can form an artificial melanin material after a chemical reaction, such as after a chemical reaction with an oxidation agent. An artificial melanin precursor can be, but is not necessarily, itself a melanin. For example, an artificial melanin precursor can be, but is not necessarily, a melanin monomer. For example, contacting artificial melanin precursors such as melanin monomers with an oxidizing agent can result in oxidative oligomerization (or, polymerization) among the artificial melanin precursors thereby forming artificial melanin material(s).

The term “selenomelanin” refers to melanin comprising selenium. For example, a selenomelanin material comprises selenium. Optionally, a chemical formula of a selenomelanin material comprises selenium (e.g., at least one selenium atom).

In certain embodiments, the term “pheomelanin” refers to a melanin whose chemical formula comprises at least one substituted or unsubstituted benzothiazine, at least one substituted or unsubstituted benzothiazole, at least one substituted or unsubstituted benzoselenazole, at least one substituted or unsubstituted benzoselenazine, at least one derivative of any of these, or any combination of these. In certain embodiments, the term pheomelanin refers to a melanin made from L-DOPA and cysteine, whose chemical formula comprises at least one substituted or unsubstituted benzothiazine, at least one substituted or unsubstituted benzothiazole, at least one substituted or unsubstituted benzoselenazole, at least one substituted or unsubstituted benzoselenazine, at least one derivative of any of these, or any combination of these. In certain embodiments, a selenium pheomelanin refers to a melanin whose chemical formula comprises at least one substituted or unsubstituted benzoselenazole, at least one substituted or unsubstituted benzoselenazine, at least one derivative of any of these, or any combination of these.

In certain embodiments, the term eumelanin refers to a melanin whose chemical formula comprises at least one dihydoxyindole (DHI) (e.g., 5,6-dihydroxyindole), at least one dihydroxyindole-2-carboxylic acid (DHICA) (e.g., 5,6-dihydroxyindole-2-carboxylic acid), or a combination of these.

The term “aging”, when used in reference to artificial melanin nanoparticles herein, refers to a process by which synthesized and isolated artificial melanin nanoparticles oxidize, and optionally further darker, over time during exposure to oxygen, such due to exposure to air. Isolated artificial melanin nanoparticles can be artificial melanin nanoparticles that are purified, such as by centrifugation, and re-dispersed in water, such as ultrapure water, or optionally another solvent or solvent solution. For example, artificial melanin nanoparticles may age if the particles are dispersed in water and are stored in a vial with the vial's top on (closed) and with the top not being opened for some extended period of time, because there is residual oxygen in the container. The aging process can alter certain properties or characteristics of artificial melanin nanoparticles, such as increasing solubility in organic solvent or decreasing toxicity to certain living biological cells. For example, without wishing to be bound by any particular theory, in some embodiments, freshly synthesized artificial melanin nanoparticles can be dynamic and shed monomers or oligomers into a cell when internalized by the cell. For example, without wishing to be bound by any particular theory, in some embodiments, freshly synthesized artificial melanin nanoparticles can be dynamic and have surface chemistry oxidation state that is not optimal for living cells when internalized by cells. For example, without wishing to be bound by any particular theory, in some embodiments, the aging process can lead to more crosslinking or otherwise chemical association between melanin compounds (monomers, oligomers) in the artificial melanin nanoparticles, potentially leading to reduced cytotoxicity, such as due to reduced shedding of melanin compounds into the cell and/or altering or stabilizing of the particles' surface chemistry.

The term “size characteristic” refers to a property, or set of properties, of a particle that directly or indirectly relates to a size attribute. According to some embodiments, a size characteristic corresponds to an empirically-derived size characteristic of a particle(s) being detected, such as a size characteristic based on, determined by, or corresponding to data from any technique or instrument that may be used to determine a particle size, such as electron microscope (e.g., SEM and TEM) or a light scattering technique (e.g., DLS). For example, a size characteristic can correspond to a spherical particle exhibiting similar or substantially same properties, such as aerodynamic, hydrodynamic, optical, and/or electrical properties, as the particle(s) being detected). According to some embodiments, a size characteristic corresponds to a physical dimension, such as a cross-sectional size (e.g., length, width, thickness, or diameter).

The term “particles” refers to small solid objects that may be dispersed and/or suspended in a fluid (e.g., liquid). For example, a slurry, a dispersion, and a suspension each include particles in a fluid. The terms “particle” and “particulate” may be used interchangeably. An exemplary particle is an artificial melanin nanoparticle. A plurality of particles may be associated together to form an agglomerate of particles. Generally, the term “particle”, such as “nanoparticle” or “melanin nanoparticle”, refers to an individual particle rather than to an agglomerate of such individual particles.

The term “dispersed” refers to species, such as particles, in a fluid forming a dispersion. As used herein, the term “dispersion” broadly refers to a mixture of one or more chemical species, such as particles, in a fluid, such as the art-recognized meaning of solution, dispersion, and/or suspension. The chemical species, such as particles, dispersed in a dispersion can be referred as a dispersed species. Optionally, a dispersion is a mixture of particles, such as artificial melanin particles, in a liquid, such as a solvent. Optionally, but not necessarily, a dispersion is a homogeneous mixture. In the context of a dispersion, the term “homogeneous” refers to a liquid mixture that appears uniform to the naked eye. In contrast, a heterogenous liquid mixture includes particles that are precipitated from or suspended in the liquid mixture and are large enough to be distinctly identifiable by the naked eye in the liquid mixture. A heterogeneous liquid mixture includes, for example, sedimented and/or sedimenting particles. Optionally, but not necessarily, the term “dispersion” is broadly intended to include solutions and dispersions, such as colloids, which are not heterogenous liquid mixtures. Optionally, but not necessarily, a dispersion is a microscopically homogenous, or uniform, mixture of particles in a liquid, such as a solvent. Optionally, but not necessarily, a dispersion is thermodynamically favored remain stably dispersed or is thermodynamically favored to segregate by sedimentation but wherein sedimentation is kinetically slowed or prevented. Particles, of a dispersion, that are characterized as stably dispersed remain dispersed in the dispersion and do not sediment or precipitate out of the liquid, of the dispersion, for at least 5 hours, preferably at least 12 hours, preferably at least 24 hours, and more preferably at least 1 week, under normal temperature and pressure (NTP) and exposure to air. In embodiments, particles that are not or cannot be dispersed in a fluid refer to particles that form precipitates or sediments upon being mixed in the fluid.

The term “size stable” refers to stability of particles in a dispersion with respect to a size characteristic of said particles. Optionally, particles in a dispersion characterized as size stable are characterized by a size characteristic being within 50%, within 40%, within 30%, preferably within 20%, more preferably within 15%, still more preferably within 10%, further more preferably within 5%, or equivalent to a reference or initial size characteristic, under given conditions and optionally for a given time. For example, nanoparticles of a dispersion characterized as size-stable in the dispersion having a pH of at least 11, with respect to an average size of the nanoparticle in the dispersion having a pH of 7, have an average size in the pH 11 dispersion that is within 50%, within 40%, within 30%, preferably within 20%, more preferably within 15%, still more preferably within 10%, further more preferably within 5%, or equivalent to an average size of the otherwise equivalent nanoparticles in the otherwise equivalent dispersion having a pH of 7. Optionally, but not necessarily, nanoparticles characterized as size stable as so size stable for time that is at least 1 hour to 5 hours, preferably at least 5 hours to 12 hours, more preferably at least 12 hours to 1 week, still more preferably at least 1 week.

The term “strong oxidizing agent” refers to a substance (e.g., compound, molecule, material) having a greater ability for subtracting, removing, or accepting one or more electron from another other substance compared to oxygen gas, including oxygen gas dissolved in a solution. The greater ability may be due to thermodynamic, kinetic, and/or electrochemical characteristics thereof. Optionally, a strong oxidizing agent has a greater or more positive standard electrode potential than O₂.

The term “U” in a unit of concentration, such as “U/mL”, refers to “unit of activity” and is a known term of art referring to enzyme catalytic activity. A unit “U” refers to the amount of enzyme that catalyzes the conversion of 1 micromole (μmole) of a substrate per minute. Thus, 1 enzyme unit (U)=1 μmol/min, where μmol refers to the amount of substrate converted. Because each enzyme has a unique substrate, a unit of activity is different for one enzyme versus another.

The term “structural color” refers to the generation of color due to interference of visible light structural features, such as a film or layer or a microstructured surface. A layer of melanin nanoparticles may exhibit color due to interference of visible light with the microstructure of the layer, rather than solely due to pigmentation. Without wishing to be bound by any particular theory, the effect of structural color can enable a spectrum on non-fading, non-photobleaching colors which can be iridescent or non-iridescent. Without wishing to be bound by any particular theory, high refractive index of melanin and synthetic melanin, and its broadband absorption across the visible spectrum allows it to interact with light in such a way that a multitude of colors are produced.

The term “peak size” size refers to the statistical mode, or peak frequency, of a particle size distribution, or the particle size most commonly found in the particle size distribution. A particle size distribution can be measured using dynamic light scattering, for example.

The term “sphere” as used herein, in the usual and customary sense, refers to a round or substantially round geometrical object in three-dimensional space that is substantially the surface of a completely round ball, analogous to a circular object in two dimensions. A sphere may be defined mathematically as the set of points that are all at the same or substantially all at the same distance r from a given point, but in three-dimensional space, where r is the radius of the mathematical ball and the given point is the center or substantially the center of the mathematical ball. In embodiments, the longest straight line through the ball, connecting two points of the sphere, passes through the center and its length is thus twice the radius; it is a diameter of the ball. A nanosphere is a nanoparticle having a radius of less than 1 μm.

The phrases “ultraviolet induced damage”, “UV induced damage”, “damage at least partially caused by an exposure to UV irradiation”, and like terms and phrases, as used interchangeably herein refer, in the usual and customary sense, to chemical changes attending irradiation of light of sufficient energy. UV induced damage can include scission of nucleic acids (e.g., DNA or RNA), and breaking of bonds in proteins, lipids, and other physiological molecules. For example, the damage can be damage resulting from reactive oxygen species (ROS).

The terms “reactive oxygen species” and “ROS” as used interchangeably herein refer, in the usual and customary sense, to transient species, typically formed during exposure to radiation (e.g., UV irradiation) capable of inducing oxidative decomposition.

The terms “cell” and “biological cell” are used interchangeably are refer to a cell carrying out metabolic or other function sufficient to preserve or replicate its genomic DNA. A cell can be identified by well-known methods in the art including, for example, presence of an intact membrane, staining by a particular dye, ability to produce progeny or, in the case of a gamete, ability to combine with a second gamete to produce a viable offspring. Cells may include prokaryotic and eukaryotic cells. Prokaryotic cells include but are not limited to bacteria. Eukaryotic cells include but are not limited to yeast cells and cells derived from plants and animals, for example mammalian, insect (e.g., Spodoptera) and human cells. A “viable cell” is a living biological cell.

The term “self-assembly” refers to a process in which individual elements assemble into a network or organized structure without external direction. In an embodiment, self-assembly leads to a decrease in entropy of a system. In an embodiment, self-assembly may be induced, or initiated, via contacting or reacting the individual elements, optionally at a certain critical concentration, and/or via temperature and/or via pressure. A “self-assembled structure” is a structure or network formed by self-assembly. In an embodiment, self-assembly is a polymer crystallization process. The Gibbs free energy of the self-assembled structure is lower than of the sum of the individual components in their non-organized arrangement prior to self-assembly under otherwise identical conditions (e.g., temperature and pressure). In an embodiment, entropy of a self-assembled structure is lower than that of the sum of the individual components in their non-organized arrangement prior to self-assembly under otherwise identical conditions (e.g., temperature and pressure). For example, artificial melanin nanoparticles of this disclosure can form by self-assembly of a plurality of oligomers and/or melanin monomers. For example, structures or layers (e.g., films) for artificial melanin nanoparticles may form by self-assembly, such as structures or layers formed of artificial melanin nanoparticles and exhibiting structural color.

The term “substantially” refers to a property, condition, or value that is within 20%, 10%, within 5%, within 1%, optionally within 0.1%, or is equivalent to a reference property, condition, or value. The term “substantially equal”, “substantially equivalent”, or “substantially unchanged”, when used in conjunction with a reference value describing a property or condition, refers to a value that is within 20%, within 10%, optionally within 5%, optionally within 1%, optionally within 0.1%, or optionally is equivalent to the provided reference value. For example, a diameter is substantially equal to 100 nm (or, “is substantially 100 nm”) if the value of the diameter is within 20%, optionally within 10%, optionally within 5%, optionally within 1%, within 0.1%, or optionally equal to 100 nm. The term “substantially greater”, when used in conjunction with a reference value describing a property or condition, refers to a value that is at least 1%, optionally at least 5%, optionally at least 10%, or optionally at least 20% greater than the provided reference value. The term “substantially less”, when used in conjunction with a reference value describing a property or condition, refers to a value that is at least 1%, optionally at least 5%, optionally at least 10%, or optionally at least 20% less than the provided reference value.

The terms “keratinocyte” and “keratinocytes” as used herein, refer to the predominant cell type in the epidermis, the outermost layer of the skin, constituting the majority (e.g., 90%-95%) of the cells found there. Keratinocytes are found in the deepest basal layer of the stratified epithelium that comprises the epidermis, and are sometimes referred to as basal cells or basal keratinocytes. Keratinocytes are maintained at various stages of differentiation in the epidermis and are responsible for forming tight junctions with the nerves of the skin. They also keep Langerhans cells of the epidermis and lymphocytes of the dermis in place. Keratinocytes contribute to protecting the body from UV radiation by taking up melanosomes. Keratinocytes contribute to protecting the body from UV radiation by taking up melanosomes, vesicles containing the endogenous photoprotectant melanin, from epidermal melanocytes. Each melanocyte in the epidermis has several dendrites that stretch out to connect it with many keratinocytes. The melanin is then stored within keratinocytes and melanocytes in the perinuclear area as “supranuclear caps”, where it protects the DNA from UV-induced damage. In addition to their structural role, keratinocytes play a role in immune system function. The skin is the first line of defense and keratinocytes serve as a barrier between an organism and its environment. In addition to preventing toxins and pathogens from entering an organisms body, they prevent the loss of moisture, heat and other important constituents of the body. In addition to their physical role, keratinocytes serve a chemical immune role as immunomodulaters, responsible for secreting inhibitory cytokines in the absence of injury and stimulating inflammation and activating Langerhans cells in response to injury. Langerhans cells serve as antigen-presenting cells when there is a skin infection and are the first cells to process microbial antigens entering the body from a skin breach.

The terms “under conditions suitable to afford uptake”, “taken up” and “take up” as used herein, refer, in the usual and customary sense, to experimental conditions well known in the art which allow uptake (e.g., endocytosis) of a species into a cell. In some embodiments, the term “internalized” when referring to particles internalized in or by a biological cell, refers to particles taken up by the biological cell, such as by, but not limited to, formation of perinuclear caps.

The term “endocytosis” as used herein, refers to a form of active transport in which a cell transports molecules (such as proteins) into the cell by engulfing them in an energy-using process. Endocytosis includes pinocytosis and phagocytosis. Pinocytosis is a mode of endocytosis in which small particles are brought into the cell, forming an invagination, and then suspended within small vesicles. These pinocytotic vesicles subsequently fuse with lysosomes to hydrolyze (break down) the particles. Phagocytosis is the process by which a cell engulfs a solid particle to form an internal compartment known as a phagosome.

The terms “treating” or “treatment” as used herein, refers to any indicia of success in the treatment or amelioration of an injury, disease, pathology or condition, including any objective or subjective parameter such as abatement; remission; diminishing of symptoms or making the injury, pathology or condition more tolerable to the patient; slowing in the rate of degeneration or decline; making the final point of degeneration less debilitating; improving a patient's physical or mental well-being. The treatment or amelioration of symptoms can be based on objective or subjective parameters; including the results of a physical examination, neuropsychiatric exams, and/or a psychiatric evaluation. The term “treating,” and conjugations thereof, include prevention of an injury, pathology, condition, or disease.

The term “effective amount” as used herein, refers to an amount sufficient to accomplish a stated purpose (e.g. Achieve the effect for which it is administered, treat a disease, reduce one or more symptoms of a disease or condition, and the like). An example of an “effective amount” is an amount sufficient to contribute to the treatment, prevention, or reduction of a symptom or symptoms of a disease, which could also be referred to as a “therapeutically effective amount.” A “reduction” of a symptom or symptoms (and grammatical equivalents of this phrase) means decreasing of the severity or frequency of the symptom(s), or elimination of the symptom(s). A “prophylactically effective amount” of a drug is an amount of a drug that, when administered to a subject, will have the intended prophylactic effect, e.g., preventing or delaying the onset (or reoccurrence) of an injury, disease, pathology or condition, or reducing the likelihood of the onset (or reoccurrence) of an injury, disease, pathology, or condition, or their symptoms. The full prophylactic effect does not necessarily occur by administration of one dose, and may occur only after administration of a series of doses. Thus, a prophylactically effective amount may be administered in one or more administrations. The exact amounts will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

The term “administering” as used herein, refers to oral administration, administration as an inhaled aerosol or as an inhaled dry powder, suppository, topical contact, intravenous, parenteral, intraperitoneal, intramuscular, intralesional, intrathecal, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. By “co-administer” it is meant that a composition described herein is administered at the same time, just prior to, or just after the administration of one or more additional therapies, for example cancer therapies such as chemotherapy, hormonal therapy, radiotherapy, or immunotherapy. The compound of the invention can be administered alone or can be co-administered to the patient. Co-administration is meant to include simultaneous or sequential administration of the compound individually or in combination (more than one compound or agent). The compositions of the present invention can be delivered transdermally, by a topical route, formulated as applicator sticks, solutions, suspensions, emulsions, gels, creams, ointments, pastes, jellies, paints, powders, and aerosols. Oral preparations include tablets, pills, powder, dragees, capsules, liquids, lozenges, cachets, gels, syrups, slurries, suspensions, etc., suitable for ingestion by the patient. Solid form preparations include powders, tablets, pills, capsules, cachets, suppositories, and dispersible granules. Liquid form preparations include solutions, suspensions, and emulsions, for example, water or water/propylene glycol solutions. The compositions of the present invention may additionally include components to provide sustained release and/or comfort. Such components include high molecular weight, anionic mucomimetic polymers, gelling polysaccharides and finely-divided drug carrier substrates. These components are discussed in greater detail in U.S. Pat. Nos. 4,911,920; 5,403,841; 5,212,162; and 4,861,760. The entire contents of these patents are incorporated herein by reference in their entirety for all purposes. The compositions of the present invention can also be delivered as microspheres for slow release in the body. For example, microspheres can be administered via intradermal injection of drug-containing microspheres, which slowly release subcutaneously (see Rao, J Biomater Sci. Polym. Ed. 7:623-645, 1995; as biodegradable and injectable gel formulations (see, e.g., Gao Pharm. Res. 12:857-863, 1995); or, as microspheres for oral administration (see, e.g., Eyles, J Pharm. Pharmacol. 49:669-674, 1997). In another embodiment, the formulations of the compositions of the present invention can be delivered by the use of liposomes which fuse with the cellular membrane or are endocytosed, i.e., by employing receptor ligands attached to the liposome, that bind to surface membrane protein receptors of the cell resulting in endocytosis. By using liposomes, particularly where the liposome surface carries receptor ligands specific for target cells, or are otherwise preferentially directed to a specific organ, one can focus the delivery of the compositions of the present invention into the target cells in vivo. (See, e.g., Al-Muhammed, J. Microencapsul. 13:293306, 1996; Chonn, Curr. Opin. Biotechnol. 6:698-708, 1995; Qstio, Am. J Hasp. Pharm. 46:1576-1587, 1989).

The term “contacting” may include allowing two or more species to react, interact, and/or physically touch, wherein the two species may be, for example, a pharmaceutical composition as provided herein and a cell. In embodiments contacting includes, for example, allowing a pharmaceutical composition as described herein to interact with a cell or a patient.

The terms “analog” and “analogue” are used interchangeably and are used in accordance with their plain ordinary meaning within Chemistry and Biology and refers to a chemical compound that is structurally similar to another compound (i.e., a so-called “reference” compound) but differs in composition, e.g., in the replacement of one atom by an atom of a different element, or in the presence of a particular functional group, or the replacement of one functional group by another functional group, or the absolute stereochemistry of one or more chiral centers of the reference compound, including isomers thereof. Accordingly, an analog is a compound that is similar or comparable in function and appearance but not in structure or origin to a reference compound.

Except where otherwise specified, the term “molecular weight” refers to an average molecular weight. Except where otherwise specified, the term “average molecular weight,” refers to number-average molecular weight. Number average molecular weight is defined as the total weight of a sample volume divided by the number of molecules within the sample. As is customary and well known in the art, peak average molecular weight and weight average molecular weight may also be used to characterize the molecular weight of the distribution of polymers within a sample.

The term “weight-average molecular weight” (M_{w}) refers to the average molecular weight defined as the sum of the products of the molecular weight of each polymer molecule (Mᵢ) multiplied by its weight fraction (wᵢ): M_{w}=ΣwᵢMᵢ. As is customary and well known in the art, peak average molecular weight and number average molecular weight may also be used to characterize the molecular weight of the distribution of polymers within a sample.

The term “wt. %” or “wt %” refers to a weight percent, or a mass fraction represented as a percentage by mass. The term “at. %” or “at %” refers to an atomic percent, or an atomic ratio represented as a percentage of a type of atom with respect to total atoms in a given matter, such as a molecule, compound, material, nanoparticle, polymer, dispersion, etc.

The term “oligomerization” refers to a chemical process of converting a monomer or a mixture of monomers into an oligomer. The term “oxidative oligomerization” refers to a chemical process of oligomerization that includes chemical oxidation of one or more monomers to form an oligomer. An oligomerization is a polymerization process, wherein an oligomer is formed as a result of the polymerization.

As used herein, the term “polymer” refers to a molecule composed of repeating structural units connected by covalent chemical bonds often characterized by a number of repeating units, also referred to as base units (e.g., greater than or equal to 2 base units). As used herein, a term “polymer” is inclusive of an “oligomer” (i.e., an oligomer is a polymer; i.e., a polymer is optionally an oligomer). An “oligomer” refers to a molecule composed of repeating structural units, also referred to as base units, connected by covalent chemical bonds often characterized by a number of repeating units less such that the oligomer is a low molecular weight polymer. Optionally, but not necessarily, for example, an oligomer has equal to or less than 100 repeating units. Optionally, but not necessarily, for example, an oligomer has a lower molecular weight less than or equal to 10,000 Da. Oligomers may be the polymerization product of one or more monomer precursors. Polymerization of one or more monomers, or monomer precursors, resulting in formation of an oligomer may be referred to as oligomerization. An oligomer optionally includes 100 or less, 50 or less, 15 or less, 12 or less, 10 or less, or 5 or less repeating units (or, “base units”). An oligomer may be characterized has having a molecular weight of 10,000 Da or less, 5,000 Da or less, 1,000 Da or less, 500 Da or less, or 200 Da or less. A dimer, a trimer, a tetramer, or a pentamer is an oligomer having two, three, four, or five, respectively, repeating units, or base units. Polymers can have, for example, greater than 100 repeating units. Polymers can have, for example, a high molecular weight, such as greater than 10,000 Da, in some embodiments greater than or equal to 50,000 Da or greater than or equal to 100,000 Da. The term polymer includes homopolymers, or polymers consisting essentially of a single repeating monomer subunit. The term polymer also includes copolymers which are formed when two or more different types of monomers are linked in the same polymer. Copolymers may comprise two or more monomer subunits, and include random, block, brush, brush block, alternating, segmented, grafted, tapered and other architectures. Useful polymers include organic polymers or inorganic polymers that may be in amorphous, semi-amorphous, crystalline or semi-crystalline states. Polymer side chains capable of cross linking polymers (e.g., physical cross linking) may be useful for some applications.

An “oligomer” refers to a molecule composed of repeating structural units, also referred to as base units, connected by covalent chemical bonds often characterized by a number of repeating units less than that of a polymer (e.g., equal to or less than 100 repeating units) and a lower molecular weights (e.g. less than or equal to 10,000 Da) than polymers. Oligomers may be the polymerization product of one or more monomer precursors. Polymerization of one or more monomers, or monomer precursors, resulting in formation of an oligomer may be referred to as oligomerization. An oligomer optionally includes 100 or less, 50 or less, 15 or less, 12 or less, 10 or less, or 5 or less repeating units (or, “base units”). An oligomer may be characterized has having a molecular weight of 10,000 Da or less, 5,000 Da or less, 1,000 Da or less, 500 Da or less, or 200 Da or less. A dimer, a trimer, a tetramer, or a pentamer is an oligomer having two, three, four, or five, respectively, repeating units, or base units.

As used herein, the term “group” may refer to a functional group of a chemical compound. Groups of the present compounds refer to an atom or a collection of atoms that are a part of the compound. Groups of the present invention may be attached to other atoms of the compound via one or more covalent bonds. Groups may also be characterized with respect to their valence state. The present invention includes groups characterized as monovalent, divalent, trivalent, etc. valence states.

The term “moiety” refers to a group, such as a functional group, of a chemical compound or molecule. A moiety is a collection of atoms that are part of the chemical compound or molecule. The present invention includes moieties characterized as monovalent, divalent, trivalent, etc. valence states. Generally, but not necessarily, a moiety comprises more than one functional group.

As used herein, the term “substituted” refers to a compound wherein one or more hydrogens is replaced by another functional group, provided that the designated atom's normal valence is not exceeded. An exemplary substituent includes, but is not limited to: a halogen or halide, an alkyl, a cycloalkyl, an aryl, a heteroaryl, an acyl, an alkoxy, an alkenyl, an alkynyl, an alkylaryl, an arylene, a heteroarylene, an alkenylene, a cycloalkenylene, an alkynylene, a hydroxyl (—OH), a carbonyl (RCOR′), a sulfide (e.g., RSR′), a phosphate (ROP(═O)(OH)₂), an azo (RNNR′), a cyanate (ROCN), an amine (e.g., primary, secondary, or tertiary), an imine (RC(═NH)R′), a nitrile (RCN), a pyridinyl (or pyridyl), a diamine, a triamine, an azide, a diimine, a triimine, an amide, a diimide, or an ether (ROR′); where each of R and R′ is independently a hydrogen or a substituted or unsubstituted alkyl group, aryl group, alkenyl group, or a combination of these. Optional substituent functional groups are also described below. In some embodiments, the term substituted refers to a compound wherein each of more than one hydrogen is replaced by another functional group, such as a halogen group. For example, when the substituent is oxo (i.e., ═O), then two hydrogens on the atom are replaced. The substituent group can be any substituent group described herein. For example, substituent groups can include one or more of a hydroxyl, an amino (e.g., primary, secondary, or tertiary), an aldehyde, a carboxylic acid, an ester, an amide, a ketone, nitro, an urea, a guanidine, cyano, fluoroalkyl (e.g., trifluoromethane), halo (e.g., fluoro), aryl (e.g., phenyl), heterocyclyl or heterocyclic group (i.e., cyclic group, e.g., aromatic (e.g., heteroaryl) or non-aromatic where the cyclic group has one or more heteroatoms), oxo, or combinations thereof. Combinations of substituents and/or variables are permissible provided that the substitutions do not significantly adversely affect synthesis or use of the compound.

As used herein, the term “derivative” refers to a compound wherein an atom or functional group is replaced by another atom or functional group (e.g., a substituent function group as also described below), including, but not limited to: a hydrogen, a halogen or halide, an alkyl, a cycloalkyl, an aryl, a heteroaryl, an acyl, an alkoxy, an alkenyl, an alkynyl, an alkylaryl, an arylene, a heteroarylene, an alkenylene, a cycloalkenylene, an alkynylene, a hydroxyl (—OH), a carbonyl (RCOR′), a sulfide (e.g., RSR′), a phosphate (ROP(═O)(OH)₂), an azo (RNNR′), a cyanate (ROCN), an amine (e.g., primary, secondary, or tertiary), an imine (RC(═NH)R′), a nitrile (RCN), a pyridinyl (or pyridyl), a diamine, a triamine, an azide, a diimine, a triimine, an amide, a diimide, or an ether (ROR′); where each of R and R′ is independently a hydrogen or a substituted or unsubstituted alkyl group, aryl group, alkenyl group, or a combination of these. Optional substituent functional groups are also described below. Optionally, the term “derivative” refers to a compound wherein one or two atoms or functional groups are independently replaced by another atom or functional group. Optionally, the term derivative does not refer to or include replacement of a chalcogen atom (S, Se) that is a member of a heterocyclic group. Optionally, and unless otherwise stated, the term derivative does not refer to or include replacement of a chalcogen atom (S, Se) nor a N (nitrogen) where the chalcogen atom and the N are members same heterocyclic group. Optionally, but not necessarily, the term derivative does not include breaking a ring structure, replacement of a ring member, or removal of a ring member.

As is customary and well known in the art, hydrogen atoms in formula, are not always explicitly shown, for example, hydrogen atoms bonded to the carbon atoms of aromatic, heteroaromatic, and alicyclic rings are not always explicitly shown. The structures provided herein, for example in the context of the description of formula and schematics and structures in the drawings, are intended to convey to one of reasonable skill in the art the chemical composition of compounds of the methods and compositions of the invention, and as will be understood by one of skill in the art, the structures provided do not indicate the specific positions and/or orientations of atoms and the corresponding bond angles between atoms of these compounds.

As used herein, the terms “alkylene” and “alkylene group” are used synonymously and refer to a divalent group derived from an alkyl group as defined herein. The invention includes compounds having one or more alkylene groups. Alkylene groups in some compounds function as linking and/or spacer groups. Compounds of the invention may have substituted and/or unsubstituted C₁-C₂₀ alkylene, C₁-C₁₀ alkylene and C₁-C₅ alkylene groups, for example, as one or more linking groups (e.g. L¹-L⁶).

As used herein, the terms “cycloalkylene” and “cycloalkylene group” are used synonymously and refer to a divalent group derived from a cycloalkyl group as defined herein. The invention includes compounds having one or more cycloalkylene groups. Cycloalkyl groups in some compounds function as linking and/or spacer groups. Compounds of the invention may have substituted and/or unsubstituted C₃-C₂₀ cycloalkylene, C₃-C₁₀ cycloalkylene and C₃-C₅ cycloalkylene groups, for example, as one or more linking groups (e.g. L¹-L⁶).

As used herein, the terms “arylene” and “arylene group” are used synonymously and refer to a divalent group derived from an aryl group as defined herein. The invention includes compounds having one or more arylene groups. In some embodiments, an arylene is a divalent group derived from an aryl group by removal of hydrogen atoms from two intra-ring carbon atoms of an aromatic ring of the aryl group. Arylene groups in some compounds function as linking and/or spacer groups. Arylene groups in some compounds function as chromophore, fluorophore, aromatic antenna, dye and/or imaging groups. Compounds of the invention include substituted and/or unsubstituted C₃-C₃₀ arylene, C₃-C₂₀ arylene, C₃-C₁₀ arylene and C₁-C₅ arylene groups, for example, as one or more linking groups (e.g. L¹-L⁶).

As used herein, the terms “heteroarylene” and “heteroarylene group” are used synonymously and refer to a divalent group derived from a heteroaryl group as defined herein. The invention includes compounds having one or more heteroarylene groups. In an embodiment, a heteroarylene is a divalent group derived from a heteroaryl group by removal of hydrogen atoms from two intra-ring carbon atoms or intra-ring nitrogen atoms of a heteroaromatic or aromatic ring of the heteroaryl group. Heteroarylene groups in some compounds function as linking and/or spacer groups. Heteroarylene groups in some compounds function as chromophore, aromatic antenna, fluorophore, dye and/or imaging groups. Compounds of the invention include substituted and/or unsubstituted C₃-C₃₀ heteroarylene, C₃-C₂₀ heteroarylene, C₁-C₁₀ heteroarylene and C₃-C₅ heteroarylene groups, for example, as one or more linking groups (e.g. L¹-L⁶).

As used herein, the terms “alkenylene” and “alkenylene group” are used synonymously and refer to a divalent group derived from an alkenyl group as defined herein. The invention includes compounds having one or more alkenylene groups. Alkenylene groups in some compounds function as linking and/or spacer groups. Compounds of the invention include substituted and/or unsubstituted C₂-C₂₀ alkenylene, C₂-C₁₀ alkenylene and C₂-C₅ alkenylene groups, for example, as one or more linking groups (e.g. L¹-L⁶).

As used herein, the terms “cylcoalkenylene” and “cylcoalkenylene group” are used synonymously and refer to a divalent group derived from a cylcoalkenyl group as defined herein. The invention includes compounds having one or more cylcoalkenylene groups. Cycloalkenylene groups in some compounds function as linking and/or spacer groups. Compounds of the invention include substituted and/or unsubstituted C₃-C₂₀ cylcoalkenylene, C₃-C₁₀ cylcoalkenylene and C₃-C₅ cylcoalkenylene groups, for example, as one or more linking groups (e.g. L¹-L⁶).

As used herein, the terms “alkynylene” and “alkynylene group” are used synonymously and refer to a divalent group derived from an alkynyl group as defined herein. The invention includes compounds having one or more alkynylene groups. Alkynylene groups in some compounds function as linking and/or spacer groups. Compounds of the invention include substituted and/or unsubstituted C₂-C₂₀ alkynylene, C₂-C₁₀ alkynylene and C₂-C₅ alkynylene groups, for example, as one or more linking groups (e.g. L¹-L⁶).

As used herein, the term “halo” refers to a halogen group such as a fluoro (—F), chloro (—Cl), bromo (—Br), iodo (—I) or astato (—At).

The term “heterocyclic” refers to ring structures containing at least one other kind of atom, in addition to carbon, in the ring. Examples of such heteroatoms include nitrogen, oxygen and sulfur. Heterocyclic rings include heterocyclic alicyclic rings and heterocyclic aromatic rings. Examples of heterocyclic rings include, but are not limited to, pyrrolidinyl, piperidyl, imidazolidinyl, tetrahydrofuryl, tetrahydrothienyl, furyl, thienyl, pyridyl, quinolyl, isoquinolyl, pyridazinyl, pyrazinyl, indolyl, imidazolyl, oxazolyl, thiazolyl, pyrazolyl, pyridinyl, benzoxadiazolyl, benzothiadiazolyl, triazolyl and tetrazolyl groups. Atoms of heterocyclic rings can be bonded to a wide range of other atoms and functional groups, for example, provided as substituents.

The term “carbocyclic” refers to ring structures containing only carbon atoms in the ring. Carbon atoms of carbocyclic rings can be bonded to a wide range of other atoms and functional groups, for example, provided as substituents.

The term “alicyclic ring” refers to a ring, or plurality of fused rings, that is not an aromatic ring. Alicyclic rings include both carbocyclic and heterocyclic rings.

The term “aromatic ring” refers to a ring, or a plurality of fused rings, that includes at least one aromatic ring group. The term aromatic ring includes aromatic rings comprising carbon, hydrogen and heteroatoms. Aromatic ring includes carbocyclic and heterocyclic aromatic rings. Aromatic rings are components of aryl groups.

The term “fused ring” or “fused ring structure” refers to a plurality of alicyclic and/or aromatic rings provided in a fused ring configuration, such as fused rings that share at least two intra ring carbon atoms and/or heteroatoms.

As used herein, the term “alkoxyalkyl” refers to a substituent of the formula alkyl-O-alkyl.

As used herein, the term “polyhydroxyalkyl” refers to a substituent having from 2 to 12 carbon atoms and from 2 to 5 hydroxyl groups, such as the 2,3-dihydroxypropyl, 2,3,4-trihydroxybutyl or 2,3,4,5-tetrahydroxypentyl residue.

As used herein, the term “polyalkoxyalkyl” refers to a substituent of the formula alkyl-(alkoxy) n-alkoxy wherein n is an integer from 1 to 10, preferably 1 to 4, and more preferably for some embodiments 1 to 3.

Amino acids include glycine, alanine, valine, leucine, isoleucine, methionine, proline, phenylalanine, tryptophan, asparagine, glutamine, glycine, serine, threonine, serine, rhreonine, asparagine, glutamine, tyrosine, cysteine, lysine, arginine, histidine, aspartic acid and glutamic acid. As used herein, reference to “a side chain residue of a natural α-amino acid” specifically includes the side chains of the above-referenced amino acids. Peptides and peptide moieties, as used and described herein, comprise two or more amino acid groups connected via peptide bonds.

Amino acids and amino acid groups refer to naturally-occurring amino acids, unnatural (non-naturally occurring) amino acids, and/or combinations of these. Naturally-occurring amino acids are those encoded by the genetic code, as well as those amino acids that are later modified, e.g., hydroxyproline, γ-carboxyglutamate, and O-phosphoserine. Naturally-occurring α-amino acids include, without limitation, alanine (Ala), cysteine (Cys), aspartic acid (Asp), glutamic acid (Glu), phenylalanine (Phe), glycine (Gly), histidine (His), isoleucine (Ile), arginine (Arg), lysine (Lys), leucine (Leu), methionine (Met), asparagine (Asn), proline (Pro), glutamine (Gln), serine (Ser), threonine (Thr), valine (Val), tryptophan (Trp), tyrosine (Tyr), and combinations thereof. Stereoisomers of a naturally-occurring α-amino acids include, without limitation, D-alanine (D-Ala), D-cysteine (D-Cys), D-aspartic acid (D-Asp), D-glutamic acid (D-Glu), D-phenylalanine (D-Phe), D-histidine (D-His), D-isoleucine (D-Ile), D-arginine (D-Arg), D-lysine (D-Lys), D-leucine (D-Leu), D-methionine (D-Met), D-asparagine (D-Asn), D-proline (D-Pro), D-glutamine (D-Gln), D-serine (D-Ser), D-threonine (D-Thr), D-valine (D-Val), D-tryptophan (D-Trp), D-tyrosine (D-Tyr), and combinations thereof.

Unnatural (non-naturally occurring) amino acids include, without limitation, amino acid analogs, amino acid mimetics, synthetic amino acids, N-substituted glycines, and N-methyl amino acids in either the L- or D-configuration that function in a manner similar to the naturally-occurring amino acids. For example, “amino acid analogs” can be unnatural amino acids that have the same basic chemical structure as naturally-occurring amino acids (i.e., a carbon that is bonded to a hydrogen, a carboxyl group, an amino group) but have modified side-chain groups or modified peptide backbones, e.g., homoserine, norleucine, methionine sulfoxide, methionine methyl sulfonium. “Amino acid mimetics” refer to chemical compounds that have a structure that is different from the general chemical structure of an amino acid, but that functions in a manner similar to a naturally-occurring amino acid. Amino acids may be referred to herein by either the commonly known three letter symbols or by the one-letter symbols recommended by the IUPAC-IUB Biochemical Nomenclature Commission.

The terms “monomer unit,” “repeating monomer unit,” “repeating unit,” and “polymerized monomer” can be used interchangeably and refer to a monomeric portion of a polymer described herein which is derived from or is a product of polymerization of one individual “monomer” or “polymerizable monomer.” Each individual monomer unit of a polymer is derived from or is a product of polymerization of one polymerizable monomer. Each individual “monomer unit” or “repeating unit” of a polymer comprises one (polymerized) polymer backbone group. For example, in a polymer that comprises monomer units X and Y arranged as X—Y—X—Y—X—Y—X—Y (where each X is identical to each other X and each Y is identical to each other Y), each X and each Y is independently can be referred to as a repeating unit or monomer unit.

Alkyl groups include straight-chain, branched and cyclic alkyl groups. Alkyl groups include those having from 1 to 30 carbon atoms. Alkyl groups include small alkyl groups having 1 to 3 carbon atoms. Alkyl groups include medium length alkyl groups having from 4-10 carbon atoms. Alkyl groups include long alkyl groups having more than 10 carbon atoms, particularly those having 10-30 carbon atoms. The term cycloalkyl specifically refers to an alky group having a ring structure such as ring structure comprising 3-30 carbon atoms, optionally 3-20 carbon atoms and optionally 2-10 carbon atoms, including an alkyl group having one or more rings. Cycloalkyl groups include those having a 3-, 4-, 5-, 6-, 7-, 8-, 9- or 10-member carbon ring(s) and particularly those having a 3-, 4-, 5-, 6-, 7-, or 8-member ring(s). The carbon rings in cycloalkyl groups can also carry alkyl groups. Cycloalkyl groups can include bicyclic and tricycloalkyl groups. Alkyl groups are optionally substituted. Substituted alkyl groups include among others those which are substituted with aryl groups, which in turn can be optionally substituted. Specific alkyl groups include methyl, ethyl, n-propyl, iso-propyl, cyclopropyl, n-butyl, s-butyl, t-butyl, cyclobutyl, n-pentyl, branched-pentyl, cyclopentyl, n-hexyl, branched hexyl, and cyclohexyl groups, all of which are optionally substituted. Substituted alkyl groups include fully halogenated or semihalogenated alkyl groups, such as alkyl groups having one or more hydrogens replaced with one or more fluorine atoms, chlorine atoms, bromine atoms and/or iodine atoms. Substituted alkyl groups include fully fluorinated or semifluorinated alkyl groups, such as alkyl groups having one or more hydrogens replaced with one or more fluorine atoms. An alkoxy group is an alkyl group that has been modified by linkage to oxygen and can be represented by the formula R—O and can also be referred to as an alkyl ether group. Examples of alkoxy groups include, but are not limited to, methoxy, ethoxy, propoxy, butoxy and heptoxy. Alkoxy groups include substituted alkoxy groups wherein the alky portion of the groups is substituted as provided herein in connection with the description of alkyl groups. As used herein MeO— refers to CH₃O—. Compositions of some embodiments of the invention comprise alkyl groups as terminating groups, such as polymer backbone terminating groups and/or polymer side chain terminating groups. Substituted alkyl groups may include substitution to incorporate one or more silyl groups, for example wherein one or more carbons are replaced by Si.

Alkenyl groups include straight-chain, branched and cyclic alkenyl groups. Alkenyl groups include those having 1, 2 or more double bonds and those in which two or more of the double bonds are conjugated double bonds. Alkenyl groups include those having from 2 to 20 carbon atoms. Alkenyl groups include small alkenyl groups having 2 to 3 carbon atoms. Alkenyl groups include medium length alkenyl groups having from 4-10 carbon atoms. Alkenyl groups include long alkenyl groups having more than 10 carbon atoms, particularly those having 10-20 carbon atoms. Cycloalkenyl groups include those in which a double bond is in the ring or in an alkenyl group attached to a ring. The term cycloalkenyl specifically refers to an alkenyl group having a ring structure, including an alkenyl group having a 3-, 4-, 5-, 6-, 7-, 8-, 9- or 10-member carbon ring(s) and particularly those having a 3-, 4-, 5-, 6- or 7-member ring(s). The carbon rings in cycloalkenyl groups can also carry alkyl groups. Cycloalkenyl groups can include bicyclic and tricyclic alkenyl groups. Alkenyl groups are optionally substituted. Substituted alkenyl groups include among others those which are substituted with alkyl or aryl groups, which groups in turn can be optionally substituted. Specific alkenyl groups include ethenyl, prop-1-enyl, prop-2-enyl, cycloprop-1-enyl, but-1-enyl, but-2-enyl, cyclobut-1-enyl, cyclobut-2-enyl, pent-1-enyl, pent-2-enyl, branched pentenyl, cyclopent-1-enyl, hex-1-enyl, branched hexenyl, cyclohexenyl, all of which are optionally substituted. Substituted alkenyl groups include fully halogenated or semihalogenated alkenyl groups, such as alkenyl groups having one or more hydrogens replaced with one or more fluorine atoms, chlorine atoms, bromine atoms and/or iodine atoms. Substituted alkenyl groups include fully fluorinated or semifluorinated alkenyl groups, such as alkenyl groups having one or more hydrogen atoms replaced with one or more fluorine atoms. Compositions of some embodiments of the invention comprise alkenyl groups as terminating groups, such as polymer backbone terminating groups and/or polymer side chain terminating groups.

Aryl groups include groups having one or more 5-, 6-7-, or 8-member aromatic rings, including heterocyclic aromatic rings. The term heteroaryl specifically refers to aryl groups having at least one 5-, 6-7-, or 8-member heterocyclic aromatic rings. Aryl groups can contain one or more fused aromatic rings, including one or more fused heteroaromatic rings, and/or a combination of one or more aromatic rings and one or more nonaromatic rings that may be fused or linked via covalent bonds. Heterocyclic aromatic rings can include one or more N, O, or S atoms in the ring. Heterocyclic aromatic rings can include those with one, two or three N atoms, those with one or two O atoms, and those with one or two S atoms, or combinations of one or two or three N, O or S atoms. Aryl groups are optionally substituted. Substituted aryl groups include among others those that are substituted with alkyl or alkenyl groups, which groups in turn can be optionally substituted. Specific aryl groups include phenyl, biphenyl groups, pyrrolidinyl, imidazolidinyl, tetrahydrofuryl, tetrahydrothienyl, furyl, thienyl, pyridyl, quinolyl, isoquinolyl, pyridazinyl, pyrazinyl, indolyl, imidazolyl, oxazolyl, thiazolyl, pyrazolyl, pyridinyl, benzoxadiazolyl, benzothiadiazolyl, and naphthyl groups, all of which are optionally substituted. Substituted aryl groups include fully halogenated or semihalogenated aryl groups, such as aryl groups having one or more hydrogens replaced with one or more fluorine atoms, chlorine atoms, bromine atoms and/or iodine atoms. Substituted aryl groups include fully fluorinated or semifluorinated aryl groups, such as aryl groups having one or more hydrogens replaced with one or more fluorine atoms. Aryl groups include, but are not limited to, aromatic group-containing or heterocylic aromatic group-containing groups corresponding to any one of the following: benzene, naphthalene, naphthoquinone, diphenylmethane, fluorene, anthracene, anthraquinone, phenanthrene, tetracene, tetracenedione, pyridine, quinoline, isoquinoline, indoles, isoindole, pyrrole, imidazole, oxazole, thiazole, pyrazole, pyrazine, pyrimidine, purine, benzimidazole, furans, benzofuran, dibenzofuran, carbazole, acridine, acridone, phenanthridine, thiophene, benzothiophene, dibenzothiophene, xanthene, xanthone, flavone, coumarin, azulene or anthracycline. As used herein, a group corresponding to the groups listed above expressly includes an aromatic or heterocyclic aromatic group, including monovalent, divalent and polyvalent groups, of the aromatic and heterocyclic aromatic groups listed herein are provided in a covalently bonded configuration in the compounds of the invention at any suitable point of attachment. In embodiments, aryl groups contain between 5 and 30 carbon atoms. In embodiments, aryl groups contain one aromatic or heteroaromatic six-member ring and one or more additional five- or six-member aromatic or heteroaromatic ring. In embodiments, aryl groups contain between five and eighteen carbon atoms in the rings. Aryl groups optionally have one or more aromatic rings or heterocyclic aromatic rings having one or more electron donating groups, electron withdrawing groups and/or targeting ligands provided as substituents. Compositions of some embodiments of the invention comprise aryl groups as terminating groups, such as polymer backbone terminating groups and/or polymer side chain terminating groups.

Arylalkyl groups are alkyl groups substituted with one or more aryl groups wherein the alkyl groups optionally carry additional substituents and the aryl groups are optionally substituted. Specific alkylaryl groups are phenyl-substituted alkyl groups, e.g., phenylmethyl groups. Alkylaryl groups are alternatively described as aryl groups substituted with one or more alkyl groups wherein the alkyl groups optionally carry additional substituents and the aryl groups are optionally substituted. Specific alkylaryl groups are alkyl-substituted phenyl groups such as methylphenyl. Substituted arylalkyl groups include fully halogenated or semihalogenated arylalkyl groups, such as arylalkyl groups having one or more alkyl and/or aryl groups having one or more hydrogens replaced with one or more fluorine atoms, chlorine atoms, bromine atoms and/or iodine atoms. Compositions of some embodiments of the invention comprise arylalkyl groups as terminating groups, such as polymer backbone terminating groups and/or polymer side chain terminating groups.

As to any of the groups described herein which contain one or more substituents, it is understood that such groups do not contain any substitution or substitution patterns which are sterically impractical and/or synthetically non-feasible. In addition, the compounds of this invention include all stereochemical isomers arising from the substitution of these compounds. Optional substitution of alkyl groups includes substitution with one or more alkenyl groups, aryl groups or both, wherein the alkenyl groups or aryl groups are optionally substituted. Optional substitution of alkenyl groups includes substitution with one or more alkyl groups, aryl groups, or both, wherein the alkyl groups or aryl groups are optionally substituted. Optional substitution of aryl groups includes substitution of the aryl ring with one or more alkyl groups, alkenyl groups, or both, wherein the alkyl groups or alkenyl groups are optionally substituted.

Optional substituents for any alkyl, alkenyl and aryl group includes substitution with one or more of the following substituents, among others:
halogen, including fluorine, chlorine, bromine or iodine;
pseudohalides, including —CN;
—COOR where R is a hydrogen or an alkyl group or an aryl group and more specifically where R is a methyl, ethyl, propyl, butyl, or phenyl group all of which groups are optionally substituted;
—COR where R is a hydrogen or an alkyl group or an aryl group and more specifically where R is a methyl, ethyl, propyl, butyl, or phenyl group all of which groups are optionally substituted;
—CON(R)₂ where each R, independently of each other R, is a hydrogen or an alkyl group or an aryl group and more specifically where R is a methyl, ethyl, propyl, butyl, or phenyl group all of which groups are optionally substituted; and where R and R can form a ring which can contain one or more double bonds and can contain one or more additional carbon atoms;
—OCON(R)₂ where each R, independently of each other R, is a hydrogen or an alkyl group or an aryl group and more specifically where R is a methyl, ethyl, propyl, butyl, or phenyl group all of which groups are optionally substituted; and where R and R can form a ring which can contain one or more double bonds and can contain one or more additional carbon atoms;
—N(R)₂ where each R, independently of each other R, is a hydrogen, or an alkyl group, or an acyl group or an aryl group and more specifically where R is a methyl, ethyl, propyl, butyl, phenyl or acetyl group, all of which are optionally substituted; and where R and R can form a ring which can contain one or more double bonds and can contain one or more additional carbon atoms;
—SR, where R is hydrogen or an alkyl group or an aryl group and more specifically where R is hydrogen, methyl, ethyl, propyl, butyl, or a phenyl group, which are optionally substituted;
—SO₂R, or —SOR where R is an alkyl group or an aryl group and more specifically where R is a methyl, ethyl, propyl, butyl, or phenyl group, all of which are optionally substituted;
—OCOOR where R is an alkyl group or an aryl group;
—SO₂N(R)₂ where each R, independently of each other R, is a hydrogen, or an alkyl group, or an aryl group all of which are optionally substituted and wherein R and R can form a ring which can contain one or more double bonds and can contain one or more additional carbon atoms; and
—OR where R is H, an alkyl group, an aryl group, or an acyl group all of which are optionally substituted. In a particular example R can be an acyl yielding —OCOR″ where R″ is a hydrogen or an alkyl group or an aryl group and more specifically where R″ is methyl, ethyl, propyl, butyl, or phenyl groups all of which groups are optionally substituted.

Specific substituted alkyl groups include haloalkyl groups, particularly trihalomethyl groups and specifically trifluoromethyl groups. Specific substituted aryl groups include mono-, di-, tri, tetra- and pentahalo-substituted phenyl groups; mono-, di-, tri-, tetra-, penta-, hexa-, and hepta-halo-substituted naphthalene groups; 3- or 4-halo-substituted phenyl groups, 3- or 4-alkyl-substituted phenyl groups, 3- or 4-alkoxy-substituted phenyl groups, 3- or 4-RCO-substituted phenyl, 5- or 6-halo-substituted naphthalene groups. More specifically, substituted aryl groups include acetylphenyl groups, particularly 4-acetylphenyl groups; fluorophenyl groups, particularly 3-fluorophenyl and 4-fluorophenyl groups; chlorophenyl groups, particularly 3-chlorophenyl and 4-chlorophenyl groups; methylphenyl groups, particularly 4-methylphenyl groups; and methoxyphenyl groups, particularly 4-methoxyphenyl groups.

As to any of the above groups which contain one or more substituents, it is understood that such groups do not contain any substitution or substitution patterns which are sterically impractical and/or synthetically non-feasible.

Many of the molecules disclosed herein contain one or more ionizable groups. Ionizable groups include groups from which a proton can be removed (e.g., —COOH) or added (e.g., amines) and groups that can be quaternized (e.g., amines). All possible ionic forms of such molecules and salts thereof are intended to be included individually in the disclosure herein. With regard to salts of the compounds herein, one of ordinary skill in the art can select from among a wide variety of available counterions that are appropriate for preparation of salts of this invention for a given application. In specific applications, the selection of a given anion or cation for preparation of a salt can result in increased or decreased solubility of that salt.

The compounds of this invention can contain one or more chiral centers. Accordingly, this invention is intended to include racemic mixtures, diastereomers, enantiomers, tautomers and mixtures enriched in one or more stereoisomer. The scope of the invention as described and claimed encompasses the racemic forms of the compounds as well as the individual enantiomers and non-racemic mixtures thereof.

As used herein, the term “isomers” refers to compounds having the same number and kind of atoms, and hence the same molecular weight, but differing in respect to the structural arrangement or configuration of the atoms.

The term “tautomer,” as used herein, refers to one of two or more structural isomers which exist in equilibrium and which are readily converted from one isomeric form to another. It will be apparent to one skilled in the art that certain compounds of this invention may exist in tautomeric forms, all such tautomeric forms of the compounds being within the scope of the invention.

Unless otherwise stated, structures depicted herein are also meant to include all stereochemical forms of the structure; i.e., the R and S configurations for each asymmetric center. Therefore, single stereochemical isomers as well as enantiomeric and diastereomeric mixtures of the present compounds are within the scope of the invention.

Unless otherwise stated, structures depicted herein are also meant to include compounds which differ only in the presence of one or more isotopically enriched atoms. For example, compounds having the present structures except for the replacement of a hydrogen by a deuterium or tritium, or the replacement of a carbon by ¹³C- or ¹⁴C-enriched carbon are within the scope of this invention.

The compounds of the present invention may also contain unnatural proportions of atomic isotopes at one or more of the atoms that constitute such compounds. For example, the compounds may be radiolabeled with radioactive isotopes, such as for example tritium (³H), iodine-125 (¹²⁵I), or carbon-14 (¹⁴C). All isotopic variations of the compounds of the present invention, whether radioactive or not, are encompassed within the scope of the present invention.

The symbol “ [symbol] ” denotes the point of attachment of a chemical moiety, functional group, atom, ion, unpaired electron, or other chemical species to the represented molecule, compound, or chemical formula. For example, in the formula

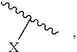

“X” represents a molecule or compound, the symbol “ [symbol] ” denotes a point of attachment of a chemical moiety, functional group, atom, ion, unpaired electron, or other chemical species to X (where X corresponds to the represented molecule, compound, or chemical formula) via covalent bonding. As used herein, the various functional groups represented will be understood to have a point of attachment at the functional group having the hyphen or dash (-) or a dash used in combination with an asterisk (*). In other words, in the case of —CH₂CH₂CH₃ or —CH₂CH₂CH₃, it will be understood that the point of attachment is the CH₂ group at the far left. If a group is recited without an asterisk or a dash, then the attachment point is indicated by the plain and ordinary meaning of the recited group.

Where substituent groups are specified by their conventional chemical formulae, written from left to right, they equally encompass the chemically identical substituents that would result from writing the structure from right to left, e.g., —CH₂O— is equivalent to —OCH₂—.

Various potentially useful and optional descriptions, background information, applications or uses of embodiments and aspects herein, terminology (to the extent not inconsistent with the terms as defined herein), mechanisms, compositions (such as artificial melanin nanoparticles and compositions and characteristics thereof), methods, techniques, measurements, calculations, definitions, and other embodiments and aspects are found in: International Patent App. No. PCT/US2017/041596 (published as International Pat. Pub. No. WO2018013609A2), International Patent App. No. PCT/US2020/039769 (published as International Pat. Pub. No. WO2021021350A3), International Patent App. No. PCT/US2020/057902 (published as International Pat. Pub. No. WO2021087076A1), International Patent App. No. PCT/US2020/057939 (published as International Pat. Pub. No. WO2021096692A1), International Patent App. No. PCT/US2023/012182 (published as International Pat. Pub. No. WO2023150205A1), International Patent App. No. PCT/US2022/026669 (published as International Pat. Pub. No. WO2022232356A1), and International Patent App. No. PCT/US2021/064842 (published as International Pat. Pub. No. WO2022140532A2), each of which is incorporated herein by reference in its entirety, to the extent not inconsistent herewith.

The term “composite” or “nanocomposite” as used herein, refers to a compound or material comprising two or more distinct components. In embodiments, a composite comprises two or more distinct components where at least one component serves as a matrix material and at least one other component serves as a performance enhancing material. In embodiments, the composite exhibits improved or altered mechanical, thermal, chemical, and/or other performance characteristics due to the interaction of the components. In embodiments, the matrix material comprises a polymer. In embodiments, the performance enhancing material comprises an additive, such as a nanoadditive. In embodiments, the performance enhancing material comprises a synthetic melanin-inspired nanoparticle.

As used herein, the term “nanomaterial” refers to a compound or material that contains one or more components with at least one dimension on the nanoscale less than 1 μm, typically ranging from 1 to 1000 nanometers, such as 1 to 500 nanometers or 1 to 250 nanometers. The nanomaterial may be composed of particles, such as nanoparticles, fibers, tubes, or other structures. In aspects, nanomaterial refers to a material comprising two or more nanoparticles. In aspects, nanomaterial refers to a material comprising two or more melanin-like nanoparticles. In aspects, a nanomaterial may be incorporated into a matrix material, such as a polymer, to enhance mechanical, chemical, and/or structural properties of the matrix material.

The term “nanoparticle” as used herein, refers to a physical particle having at least one size characteristic or physical dimension less than 1 μm, typically ranging from 1 to 1000 nanometers, such as 1 to 100 nanometers. Optionally, term “nanoparticle” as used herein, refers to a physical particle whose longest size characteristic or physical dimension is less than 1 μm, typically ranging from 1 to 1000 nanometers, such as 1 to 500 nanometers or 1 to 250 nanometers.

As used herein, the term “additive” refers to an agent, component, constituent, material, or composition that is combined with (e.g., mixed, blended, dissolved, dispersed, suspended, alloyed, compounded, coated, grafted, reacted, or otherwise introduced into) another agent, component, constituent, material, composition, formulation, or reaction mixture. Unless the context clearly indicates otherwise, an additive may be organic or inorganic, polymeric or non-polymeric, particulate or non-particulate, and may be provided in any suitable physical form. For example, in aspects, the additive is provided as a solid, a liquid, a gel, a dispersion, an emulsion, a slurry, a powder, a granule, a pellet, a solution, a concentrate, or any combination thereof. An additive may be incorporated into a system in any effective amount, including trace, minor, or major amounts. In aspects, the additive modifies, enhances, enables, or tunes one or more properties or performance characteristics of the resulting system. In this context, the terms modifies, enhances, enables, tunes, and like terms refer to any measurable or perceivable change (e.g., increase) in the performance, efficiency, or outcome of the system in relation to a baseline (i.e., the outcome of the system without the additive). In aspects, the additive is biocompatible. In aspects, the additive is biologically inert. In aspects, the additive is a nanoadditive.

The term, “nanoadditive” refers to an additive that is or comprises a nanomaterial. In aspects, the nanoadditive is incorporated into another material, composition, formulation, or reaction mixture to enhance or modify its properties or performance characteristics. In aspects, a nanoadditive is incorporated into another material or composition to improve properties such as long-term stability, chemical or biological reactivity, and/or other properties. In aspects, a nanoadditive is incorporated into a biological system (e.g., a biological reaction mixture) to improve the quality of a reaction, such as by stabilizing the reaction environment or optimizing conditions to achieve more favorable or consistent results. For example, incorporating a nanoadditive into a cell-free genetic expression system may improve or enhance free radical scavenging activity, and/or introduce broadband absorptive qualities. In aspects, the nanoadditive is incorporated into a cell-free system (such as an E. coli-based cell-free system). In aspects, the nanoadditive is characterized by broadband light absorption properties. In aspects, the nanoadditive is characterized by high radical scavenging activity. In aspects, the nanoadditive is or comprises an artificial melanin nanomaterial and/or a melanin-like nanomaterial. In aspects, the nanoadditive is or comprises an artificial allomelanin nanomaterial and/or an allomelanin-like nanomaterial.

In the context of introducing an additive (e.g., a nanoadditive) to a biological system (e.g., biological reaction, a cell-free reaction), the terms “maintain”, “protect”, “retain”, and like terms, refer to the preservation of at least one structural or functional property or characteristic (e.g., reaction stability or genetic expression) of the biological system under specific conditions (e.g., with and without UV exposure). In aspects, introducing an additive to a cell-free synthesis reaction to generate a cell-free reaction mixture, protects the reaction from damage caused by UV irradiation exposure. For example, the resulting cell-free reaction mixture retains at least 25% (e.g., at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99%, or 100%, optionally between about 25%-100% or about 25%-50%) of the synthesis activity of an unexposed cell-free synthesis tested under like conditions. In aspects, the damage is at least partially caused by an exposure to solar light, ultraviolet (UV) irradiation, gamma irradiation, x-ray irradiation, or any combination thereof. In aspects, the additive may act as a protective component by potentially introducing new properties to the resulting biological system, such as improved resistance to environmental factors (e.g., radiation and/or temperature). In the context of composites, mixtures, formulations, and/or solutions, the terms “improve”, “enhance”, and like terms refer to a measureable or perceivable (e.g., observable cracks on a surface of a material) increase or preservation in the performance of a material after it interacts, contacts, or combines with a different material in relation to a baseline. “Baseline” refers to the performance of a material before an interaction, contact, or combination with a different material. For example, contacting a polymeric material with a nanoadditive may improve or enhance the thermomechanical properties of the polymeric material by providing additional structural support, enhancing free radical scavenging activity, and/or introducing broadband absorptive qualities. For example, a protective component (e.g., a nanoparticle or nanoadditive) may disperse, adhere, or react with a matrix material component (e.g., a polymeric material), potentially introducing new properties, such as improved resistance to environmental factors (e.g., radiation and/or temperature), increased conductivity, or reduced weight. In aspects, the combination of distinct materials to generate a composite synergistically enhances the overall performance of the composite beyond what either material could achieve independently.

As used herein, “optical pigmentation” refers to observable and/or measurable optical characteristics of a material (e.g., an additive or a melanin material). In aspects, the optical characteristics of a material at least partially determines how the material appears (e.g., by human eyesight or by instrument) and/or how the material impacts optical measurements of a system in which it is present. In aspects, optical pigmentation may be expressed as, or characterized by, any one or more of: color, whiteness, blackness, hue, shade, tone, tint, brightness, lightness/darkness, opacity, transparency, translucency, haze, clarity, fluorescence, luminescence, or any combination thereof. In aspects, optical pigmentation may be expressed as, or characterized by, one or more underlying optical characteristics, including, but not limited to, absorbance, optical density, extinction, coefficient, reflectance, transmittance, and/or scattering (e.g., Mie or Rayleigh scattering). In aspects, optical pigmentation is wavelength-dependent, and may encompass interactions across one or more spectral regions including ultraviolet (UV) (e.g., UVC/UVB/UVA), visible, and/or near-infrared wavelengths. In aspects, optical pigmentation may results from any combination of intrinsic (e.g., electronic structure, bandgap, chromophores, defects) and extrinsic factors (e.g., particle size distribution, aggregation state, concentration/loading, pathlength, dispersion quality, surface coatings, and the optical properties of a surrounding reaction medium or matrix). Accordingly, in aspects, optical pigmentation may be assessed for a material in isolation and/or present in a reaction mixture or composition, including a cell-free reaction mixture, and may be described with reference to one or more detection wavelengths used to observe a fluorometric and/or colorimetric response. In aspects, optical pigmentation of a nanoadditive refers to the optical characteristics of synthetic nanoparticles and/or nanoparticle-containing dispersions, including pigmentation arising from absorption and/or scattering by the nanoparticles. In aspects, optical pigmentation includes broadband UV absorption, including at wavelength corresponding to UVC, UVB, and UVA, optionally while maintaining relatively low absorbance and/or low extinction at one or more visible or assay-detectable wavelengths. In some aspects, optical pigmentation is evaluated relative to one or more wavelengths used to observe a fluorometric and/or colorimetric response, such that a material may be described as having optical pigmentation that is “sufficiently light” when, at the relevant detection wavelength(s), the material's optical pigmentation does not prevent or substantially interfere with observation of the response. In some aspects, optical pigmentation is defined with respect to observation by unaided visual inspection and/or by instrumented detection, including, but not limited to, a camera, plate reader, spectrophotometer, photodiode-based detector, point-of-care optical reader, and is not limited to any single measure modality.

As used herein, the terms “AMNP” and “ANP” are equivalent and interchangeable and refer to artificial allomelanin nanoparticles.

As used herein, the terms “artificial” and “synthetic” in reference to melanin (e.g., “artificial melanin” and “synthetic melanin”, respectively) are equivalent and interchangeable.

As used herein, unless otherwise stated explicitly, the term “NP” refers to nanoparticle.

As used herein, the term “SMP” refers to synthetic (or artificial) melanin particle. In some aspects and examples, an SMP is a synthetic/artificial polydopamine (PDA) particle. In some aspects and examples, an SMP is a synthetic/artificial allomelanin (ANMP or ANP) particle.

As used herein, the terms “melanin-inspired” and “melanin-like” are equivalent and interchangeable to describe certain compounds and materials disclosed herein. A melanin-inspired or melanin-like compound or material refers to a compound or material that contains a structural isomer of one or more artificial melanin precursors, such as a structural isomer of one or more melanin monomers. For example, contacting a plurality of structural isomers of one or more artificial melanin precursor with an oxidizing agent can result in oxidative oligomerization (or, polymerization) among the structural isomers thereby forming a melanin-inspired material.

As used herein, the terms “allomelanin-inspired” and “allomelanin-like” are equivalent and interchangeable to describe certain compounds and materials disclosed herein. Allomelanin refers to a group of melanins that consist of nitrogen-free precursors such as catechol and 1,8-dihydroxynaphthalene (1,8-DHN). An allomelanin-inspired or allomelanin-like compound or material refers to a compound or material that contains a structural isomer of one or more allomelanin precursors, such as a structural isomer of one or more allomelanin monomers (e.g., 1,8-DHN). In some aspects, the structural isomer of the allomelanin monomer refers to 1,3-DHN, 1,4-DHN, 1,5-DHN, 1,6-DHN, 1,7-DHN, 2,3-DHN, 2,6-DHN, or 2,7-DHN. In some aspects, the structural isomer of the allomelanin monomer refers to 2,3-DHN, 2,6-DHN, or 2,7-DHN. For example, contacting a plurality of structural isomers of one or more allomelanin precursor with an oxidizing agent can result in oxidative oligomerization (or, polymerization) among the structural isomers thereby forming an allomelanin-inspired material.

In an embodiment, a composition or compound of the invention, such as an alloy or precursor to an alloy, is isolated or substantially purified. In an embodiment, an isolated or purified compound is at least partially isolated or substantially purified as would be understood in the art. In an embodiment, a substantially purified composition, compound or formulation of the invention has a chemical purity of 95%, optionally for some applications 99%, optionally for some applications 99.9%, optionally for some applications 99.99%, and optionally for some applications 99.999% pure.

### DETAILED DESCRIPTION OF THE INVENTION

Though it is often recommended to carry cell-free reactions out in a closed environment, the use of nanoparticles as additives, such as melanin nanoparticles, can reduce the need for light protective packaging and allow users in point-of-need environments to carry these reactions out in the sun without worrying about substantially impacting reaction efficiency.

In the following description, numerous specific details of the devices, device components and methods of the present invention are set forth in order to provide a thorough explanation of the precise nature of the invention. It will be apparent, however, to those of skill in the art that the invention can be practiced without these specific details.

### Aspects of the Invention

Various aspects are contemplated herein, several of which are set forth in the paragraphs below. It is explicitly contemplated that any aspect or portion thereof can be combined to form an aspect. Furthermore, although the aspects below are subdivided into aspects A, B, C, D, and so forth, it is explicitly contemplated that aspects in each of subdivisions A, B, C, D, etc. can be combined in any manner. Moreover, the term “any preceding aspect” means any aspect that appears prior to the aspect that contains such phrase (in other words, the sentence “Aspect B13: The method of any one of aspects B1-B12, or any preceding aspect, . . . ” means that any aspect prior to aspect B13 is referenced, including aspects B1-B12 and all of the “A” aspects, including any subdivision thereof (e.g., A1, A2, A3, A1a, A1b, A1c, etc.). For example, it is contemplated that, optionally, any method, polymer or composition of any of the below aspects may be useful with or combined with any other aspect provided below. Further, for example, it is contemplated that any embodiment described elsewhere herein, including above this paragraph, may optionally be combined with any of the below listed aspects. In some instances in the aspects below, or elsewhere herein, two open ended ranges are disclosed to be combinable into a range. For example, “at least X” is disclosed to be combinable with “less than Y” to form a range, in which X and Y are numeric values. For the purposes of forming ranges herein, it is explicitly contemplated that “at least X” combined with “less than Y” forms a range of X—Y inclusive of value X and value Y, even through “less than Y” in isolation does not include Y. Unless otherwise stated, ranges recited herein are explicitly contemplated to include the modifier “about” (as defined herein) for each value. For example, a range of “between 1 and 5” is intended to include a range of “between about 1 and about 5”.

Aspect A1: An additive, wherein the additive comprises a synthetic melanin nanomaterial, a synthetic melanin-like nanomaterial, or a combination thereof.

Aspect A1a: The additive of aspect A1, wherein the additive is a nanoadditive, and the nanoadditive is a nanoparticle.

Aspect A1b: An additive, wherein the additive comprises a synthetic melanin nanoparticle, wherein the synthetic melanin nanoparticle comprises a synthetic melanin oligomer or a synthetic melanin polymer.

Aspect A2: The additive of aspect A1, wherein the additive is configured to protect a cell-free reaction from damage, optionally wherein the damage is at least partially caused by an exposure to solar light, ultraviolet (UV) irradiation, gamma irradiation, x-ray irradiation, or any combination thereof.

Aspect A3: The additive of any one of the preceding aspects, wherein the synthetic melanin nanoparticle is biocompatible.

Aspect A3a: The additive of any one of aspects A1-A3, wherein the synthetic melanin nanoparticle is biologically inert.

Aspect A4: The additive of any one of aspects A2-A3, or any preceding aspect, wherein the additive is present in the cell-free reaction at a concentration of between 0.25 mg/mL and 15 mg/mL, optionally between 0.25 mg/mL and 7.5 mg/mL, optionally between 0.25 mg/mL and 5 mg/mL, optionally between 0.25 mg/mL and 3 mg/mL, and/or optionally no greater than 3 mg/mL.

Aspect A5: The additive of any one of aspects A2-A4, or any preceding aspect, wherein the exposure comprises an exposure duration of between 5 minutes and 120 minutes, optionally between 5 minutes and 90 minutes, optionally between 5 minutes and 60 minutes, optionally between 1 day and 30 days, between 1 day and 15 days, or between 1 day and 5 days.

Aspect A6: The additive of any one of aspects A2-A5, or any preceding aspect, wherein the cell-free reaction comprises a cell-free peptide synthesis reaction, a cell-free metabolic pathway reaction, a cell-free biosensor reaction, a cell-free gene expression system, an E. coli-based cell-free system, or any combination thereof.

Aspect A6a: The additive of any one of aspects A2-A6, or any preceding aspect, wherein the cell-free reaction comprises a cell-free aqueous solution.

Aspect A7: The additive of any one of aspects A1-A6, wherein the synthetic melanin nanoparticle comprises a synthetic allomelanin nanoparticle, a synthetic eumelanin nanoparticle, a synthetic polydopamine nanoparticle, a synthetic selenomelanin nanoparticle, a synthetic pheomelanin, or any combination thereof.

Aspect A8: The additive of any one of aspects A1-A7, wherein the synthetic melanin nanoparticle comprises a synthetic allomelanin nanoparticle, a synthetic eumelanin nanoparticle, a synthetic polydopamine nanoparticle, or any combination thereof.

Aspect A9: The additive of any one of aspects A1-A8, wherein the synthetic melanin oligomer or synthetic melanin polymer comprises a plurality of covalently-bonded melanin base units.

Aspect A10: The additive of aspect A9, or any preceding aspect, wherein the plurality of covalently-bonded melanin base units comprises 3,4-dihydroxydopamine monomer units, 3,4-dioxydopamine monomer units, 3,4-dihydroxynaphthalene monomer units, 1,3-dihydroxynapthalene monomer units, 1,4-dihydroxynapthalene monomer units, 1,5-dihydroxynapthalene monomer units, 1,6-dihydroxynapthalene monomer units, 1,7-dihydroxynapthalene monomer units, 1,8-dihydroxynapthalene monomer units, 2,3-dihydroxynapthalene monomer units, 2,6-dihydroxynapthalene monomer units, 2,7-dihydroxynapthalene monomer units, I-3,4-dihydroxyphenylalanine monomer units, catechol units, or any combination of these.

Aspect A11: The additive of aspect A9 or A10, or any preceding aspect, wherein the plurality of covalently-bonded melanin base units comprises a nitrogen-free precursor of allomelanin, optionally catechol units, 1,8-dihydroxynapthalene monomer units or an isomer thereof, or a combination thereof.

Aspect A12: The additive of any one of aspect A9-A11, or any preceding aspect, wherein the plurality of covalently-bonded melanin base units comprises catechol units, 2,3-DHN monomer units, 2,6-DHN monomer units, 2,7-DHN monomer units, or any combination thereof.

Aspect A13: The additive of any one of aspects A8-A12, or any preceding aspect, wherein the plurality of covalently-bonded melanin base units comprises a 1:1 molar ratio of 1,8-dihydroxynapthalene: 2,3-dihydroxynapthalene monomer units.

Aspect A14: The additive of any one of aspects A1-A13, wherein the synthetic melanin oligomer or synthetic melanin polymer comprises a homopolymer or a copolymer, optionally wherein the oligomer or polymer comprises a random copolymer.

Aspect A15: The additive of aspect A14, or any preceding aspect, wherein the homopolymer comprises a 2,3 DHN homopolymer or a 2,6-DHN homopolymer.

Aspect A16: The additive of aspect A14 or A15, or any preceding aspect, wherein the homopolymer comprises a 2,6-DHN homopolymer.

Aspect A17: The additive of any one of aspects A14-A16, or any preceding aspect, wherein the copolymer comprises two or more monomers selected from the group consisting of: catechol, 2,7-DHN, 2,6-DHN, and 2,3-DHN.

Aspect A18: The additive of any one of aspects A14-A17, or any preceding aspect, wherein the copolymer comprises 2,3-DHN and 2,7-DHN.

Aspect A19: The additive of any one of aspects A14-A18, or any preceding aspect, wherein the copolymer comprises 2,6-DHN and 2,7-DHN.

Aspect A20: The additive of any one of aspects A14-A19, or any preceding aspect, wherein the copolymer comprises catechol and 2,3-DHN.

Aspect A21: The additive of any one of aspects A14-A20, or any preceding aspect, wherein the copolymer comprises catechol monomer units and 2,6-DHN monomer units, optionally wherein the copolymer comprises between about 1-30 wt % (e.g., between about 1-30 wt %, between about 1-25 wt %, between about 1-20 wt %, between about 5-30 wt %, between about 5-25 wt %, between about 5-20 wt %, between about 10-30 wt %, between about 10-20 wt %) of catechol and about 70-99 wt % of 2,6-DHN, optionally wherein the copolymer comprises about 16.7 wt % of catechol and about 83.3 wt % of 2,6-DHN.

Aspect A21a: The additive of any one of aspects A14-A20, or any preceding aspect, wherein the copolymer comprises catechol monomer units and 2,6-DHN monomer units, optionally wherein the copolymer comprises between about 10-20 wt % and about 80-90 wt % of 2,6-DHN, optionally wherein the copolymer comprises about 16.7 wt % of catechol and about 83.3 wt % of 2,6-DHN.

Aspect B1: A method for protecting a cell-free reaction from damage, the method comprising contacting a cell-free reaction with an additive to generate a cell-free reaction mixture, wherein the additive comprises a synthetic melanin nanomaterial, a synthetic melanin-like nanomaterial, or a combination thereof.

Aspect B1a: A method for protecting a cell-free reaction from damage, the method comprising contacting a cell-free reaction with an additive, wherein the additive comprises a synthetic melanin nanoparticle, wherein the synthetic melanin nanoparticle comprises a synthetic melanin oligomer or a synthetic melanin polymer.

Aspect B2: The method of aspect B1, or any preceding aspect, wherein the damage is at least partially caused by an exposure to solar light or ultraviolet (UV) irradiation.

Aspect B3: The method of aspect B1 or B2, or any preceding aspect, wherein the cell-free reaction comprises a cell-free peptide synthesis reaction, a cell-free metabolic pathway reaction, a cell-free biosensor reaction, a cell-free gene expression system, an E. coli-based cell-free system, or any combination thereof.

Aspect B4: The method of any one of aspects B1-B3, or any preceding aspect, wherein the synthetic melanin nanoparticle comprises a synthetic allomelanin nanoparticle, a synthetic eumelanin nanoparticle, a synthetic polydopamine nanoparticle, a synthetic selenomelanin nanoparticle, a synthetic pheomelanin, or any combination thereof.

Aspect B5: The method of any one of aspects B1-B4, or any preceding aspect, wherein the synthetic melanin nanoparticle comprises a synthetic allomelanin nanoparticle, a synthetic eumelanin nanoparticle, a synthetic polydopamine nanoparticle, or any combination thereof.

Aspect B6: The method of any one of aspects B1-B5, or any preceding aspect, wherein the synthetic melanin oligomer or polymer comprises a plurality of covalently-bonded melanin base units.

Aspect B7: The method of aspect B6, or any preceding aspect, wherein the plurality of covalently-bonded melanin base units 3,4-dihydroxydopamine monomer units, 3,4-dioxydopamine monomer units, 3,4-dihydroxynaphthalene monomer units, 1,3-dihydroxynapthalene monomer units, 1,4-dihydroxynapthalene monomer units, 1,5-dihydroxynapthalene monomer units, 1,6-dihydroxynapthalene monomer units, 1,7-dihydroxynapthalene monomer units, 1,8-dihydroxynapthalene monomer units, 2,3-dihydroxynapthalene monomer units, 2,6-dihydroxynapthalene monomer units, 2,7-dihydroxynapthalene monomer units, I-3,4-dihydroxyphenylalanine monomer units, catechol units, or any combination of these.

Aspect B8: The method of aspect B6 or B7, or any preceding aspect, wherein the plurality of covalently-bonded melanin base units comprises a nitrogen-free precursor of allomelanin, optionally catechol units, 1,8-dihydroxynapthalene monomer units or an isomer thereof, or a combination thereof.

Aspect B9: The method of any one of aspects B6-B8, or any preceding aspect, wherein the plurality of covalently-bonded melanin base units comprises about 1:1 molar ratio of 1,8-dihydroxynapthalene: 2,3-dihydroxynapthalene monomer units.

Aspect B9a: The method of any one of aspects B6-B8, or any preceding aspect, wherein the plurality of covalently-bonded melanin base units comprises a 1:1 molar ratio of 1,8-dihydroxynapthalene: 2,3-dihydroxynapthalene monomer units.

Aspect B10: The method of any one of B6-B9, or any preceding aspect, wherein the plurality of covalently-bonded melanin base units comprises catechol units, 2,3-DHN monomer units, 2,6-DHN monomer units, 2,7-DHN monomer units, or any combination thereof.

Aspect B11: The method of any one of aspects B6-B10, or any preceding aspect, wherein the plurality of covalently-bonded melanin base units comprises about a 1:1 molar ratio of 2,7-dihydroxynapthalene: 2,3-dihydroxynapthalene monomer units.

Aspect B11a: The method of any one of aspects B6-B10, or any preceding aspect, wherein the plurality of covalently-bonded melanin base units comprises a 1:1 molar ratio of 2,7-dihydroxynapthalene: 2,3-dihydroxynapthalene monomer units.

Aspect B12: The method of any one of aspects B1-B11, or any preceding aspect, wherein the synthetic melanin oligomer or synthetic melanin polymer comprises a homopolymer or a copolymer.

Aspect B13: The method of aspect B12, or any preceding aspect, wherein the homopolymer comprises a 2,3 DHN homopolymer or a 2,6-DHN homopolymer.

Aspect B14: The method of aspect B12 or B13, or any preceding aspect, wherein the homopolymer comprises a 2,6-DHN homopolymer.

Aspect B14a: The method of aspect B12 or B13, or any preceding aspect, wherein the homopolymer comprises a 2,3-DHN homopolymer.

Aspect B15: The method of any one of aspects B12-B14, or any preceding aspect, wherein the copolymer comprises two or more monomers selected from the group consisting of: catechol, 2,7-DHN, 2,6-DHN, and 2,3-DHN.

Aspect B16: The method of any one of aspects B12-B15, or any preceding aspect, wherein the copolymer comprises 2,3-DHN and 2,7-DHN.

Aspect B16a: The method of any one of aspects B12-B15, or any preceding aspect, wherein the copolymer comprises about a 1:1 molar ratio of 2,3-DHN: 2,7-DHN monomer units.

Aspect B17: The method of any one of aspects B12-B16, or any preceding aspect, wherein the copolymer comprises 2,6-DHN and 2,7-DHN.

Aspect B17a: The method of any one of aspects B12-B16, or any preceding aspect, wherein the copolymer comprises about a 1:1 molar ratio of 2,6-DHN: 2,7-DHN monomer units.

Aspect B18: The method of any one of aspects B12-B17, or any preceding aspect, wherein the copolymer comprises catechol and 2,3-DHN.

Aspect B18a: The method of any one of aspects B12-B17, or any preceding aspect, wherein the copolymer comprises between about a 1:1 molar ratio and about a 1:10 molar ratio of catechol: 2,3-DHN monomer units (e.g., about a 1:1 molar ratio, about a 1:2 molar ratio, about a 1:3 molar ratio, about a 1:4 molar ratio, about a 1:5 molar ratio, about a 1:6 molar ratio, about a 1:7 molar ratio, about a 1:8 molar ratio, about a 1:9 molar ratio, or about a 1:10 molar ratio of catechol: 2,3-DHN monomer units).

Aspect B18b: The method of any one of aspects B12-B17, or any preceding aspect, wherein the copolymer comprises about a 1:5 molar ratio of catechol: 2,3-DHN monomer units.

Aspect B19: The method of any one of aspects B12-B18, or any preceding aspect, wherein the copolymer comprises catechol and 2,6-DHN.

Aspect B19a: The method of any one of aspects B12-B18, or any preceding aspect, wherein the copolymer comprises between about a 1:1 molar ratio and about a 1:10 molar ratio of catechol: 2,6-DHN monomer units (e.g., about a 1:1 molar ratio, about a 1:2 molar ratio, about a 1:3 molar ratio, about a 1:4 molar ratio, about a 1:5 molar ratio, about a 1:6 molar ratio, about a 1:7 molar ratio, about a 1:8 molar ratio, about a 1:9 molar ratio, or about a 1:10 molar ratio of catechol: 2,6-DHN monomer units).

Aspect B19b: The method of any one of aspects B12-B18, or any preceding aspect, wherein the copolymer comprises about a 1:5 molar ratio of catechol: 2,6-DHN monomer units.

Aspect B20: The method of any one of aspects B1-B19, or any preceding aspect, wherein the method further comprises freeze-drying the cell-free reaction after contacting the cell-free reaction with the additive.

Aspect B21: The method of any one of aspects B1-B20, or any preceding aspect, wherein the method further comprises observing an optical change in the color of the cell-free reaction.

Aspect B21a: The method of aspect B21, or any preceding aspect, wherein the observing an optical change comprises the use of a biosensor, optionally a plate reader assay.

Aspect B22: The method of aspect B21, or any preceding aspect, wherein the method does not comprise removing the additive to observe the optical change, optionally wherein the method does not comprise removing the additive by a centrifugation step or a filtration step to observe the optical change.

Aspect B23: The method of any one of aspects B1-B22, or any preceding aspect, wherein the method results in an observable increase in the visible light absorption of the cell-free reaction.

Aspect B24: The method of any one of aspects B1-B23, or any preceding aspect, wherein the cell-free reaction comprises a cell-free gene expression system.

Aspect B24a: The method of aspect B23, or any preceding aspect, wherein the damage is caused by an exposure to solar light or ultraviolet (UV) irradiation, and wherein the method results in preserving at least 30% (e.g., at least 30%, at least 35%, at least 40%, at least 45%, at least 50%, at least 55%, at least 60%, at least 65%, at least 70%, at least 75%, at least 80%, at least 85%, at least 90%, at least 95%, at least 99%, or 100%, optionally less than 150%, optionally less than 125%) of the genetic expression levels of the cell-free reaction prior the exposure to solar light or UV irradiation.

Aspect C1: A method for protecting a cell-free reaction from damage, optionally wherein the damage is caused by an exposure to solar light or ultraviolet (UV) irradiation, the method comprising contacting a cell-free reaction with the additive of any one of aspects A1-A21, or any preceding aspect.

Aspect D1: A cell-free reaction mixture comprising:
a cell-free reaction system; and
an additive comprising a synthetic melanin nanomaterial, a synthetic melanin-like nanomaterial, or a combination thereof, optionally wherein the additive is the additive of any one of the preceding claims,
   wherein the additive has an optical pigmentation that is sufficiently light at one or more detection wavelengths associated with a fluorometric response, a colorimetric response, or a combination thereof, of the cell-free reaction system such that, when present in the reaction mixture, the additive does not prevent or substantially interfere with observation of said fluorometric and/or colorimetric response.

Aspect D2: The cell-free reaction mixture of aspect D1, or any preceding aspect, wherein “does not substantially interfere” means that, relative to an otherwise identical reaction mixture lacking the additive, the additive does not cause a material reduction in at least one of:
(i) a signal-to-background ratio;
(ii) a signal-to-noise ratio; or
(iii) an ability to visually distinguish a change corresponding to the fluorometric response and/or the colorimetric response; or
(iv) any combination of (i)-(iii).

Aspect D3: The cell-free reaction mixture of aspect D1 or D2, or any preceding aspect, wherein the fluorometric response and/or the colorimetric response is observable without performing an additional purification step, filtration step, or centrifugation step to remove the additive from the reaction mixture prior to said observation.

Aspect D4: The cell-free reaction mixture of any one of aspects D1-D3, or any preceding aspect, wherein the additive exhibits broadband absorption in the ultraviolet (UV) range.

Aspect D4a: The cell-free reaction mixture of any one of aspects D1-D3, or any preceding aspect, wherein the additive exhibits broadband absorption in the ultraviolet (UV) range, including at least one of:
(i) UVC (200-280 nm);
(ii) UVB (280-230 nm);
(iii) UVA (320-400 nm); or
(iv) any combination of (i)-(iii).

Aspect D5: The cell-free reaction mixture of any one of aspects D1-D4, or any preceding aspect, wherein the additive has radical scavenging activity effective to quench free radicals generated by exposure of the reaction ionizing radiation, UV radiation, or a combination thereof.

Aspect D6: The cell-free reaction mixture of any one of aspects D1-D5, or any preceding aspect, wherein additive comprises a synthetic melanin nanoparticle comprising a synthetic allomelanin nanoparticle, a synthetic eumelanin nanoparticle, a synthetic polydopamine nanoparticle, or any combination thereof.

Aspect E1: The additive of any preceding aspect, wherein the additive exhibits broadband absorption in the ultraviolet (UV) range.

Aspect E1a: The additive of any preceding aspect, wherein the additive exhibits broadband absorption in the ultraviolet (UV) range, including at least one of:
(v) UVC (200-280 nm);
(vi) UVB (280-230 nm);
(vii) UVA (320-400 nm); or
(viii) any combination of (i)-(iii).

Aspect E2: The additive of any preceding aspect, wherein the additive has radical scavenging activity effective to quench free radicals generated by exposure of the reaction ionizing radiation, UV radiation, or a combination thereof.

The invention can be further understood by the following non-limiting examples.

### Example 1

Melanin is a biological black-brown pigment found in a variety of living organisms. There are various classes of melanin in nature including eumelanin, pheomelanin, neuromelanin, pyomelanin, and allomelanin.¹ Natural melanin derivatives such as eumelanin, pheomelanin, and pyomelanin are derived from tyrosine metabolic pathways that convert the amino acid into different melanogenic derivatives with tyrosinase and laccase enzymes or deamination and oxidation in the case of pyomelanins.²⁻⁴ Allomelanins, which are dihydroxynaphthalene derivatives can be produced from malonyl-CoA through polyketide synthase pathways.⁴ Levodopa (L-DOPA) is a particularly well studied eumelanin derived from tyrosine that has been biologically produced both natively and through metabolic engineering efforts in a variety of organisms including E. coli.⁵⁻⁸ Production of these melanin's have recently been used in synthetic biology for self-pigmenting textiles.⁹ Melanin's characteristic property is its radiation protective qualities that results from its ability to sequester free radicals generated from radiation and conversion of light-radiation to heat via its prominent photothermal properties. These hallmark properties of melanin benefit melanized organisms through various mechanisms including protection from radiation¹⁰⁻¹³, thermoregulation¹⁴, immunoprotection¹⁵ mechanical support¹⁶, and exposure chemical insult.^{17,18}

The favorable properties of natural melanins have motivated the development of synthetic analogues with controlled, uniform morphologies. For instance, synthetic analogues of eumelanin including polydopamine (PDA) and L-DOPA nanoparticles have been used in the literature for their radical scavenging activity and photothermal properties.¹⁹ PDA nanoparticles have been shown to be biocompatible and act as a wound healing agent by rescuing superoxide dismutase through its antioxidant activity.¹⁸ Synthetic allomelanin analogues derived from oxidatively polymerizing 1,8 dihydroxynapthalene (1,8 DHN) have also been developed and used for their radical scavenging activity as well as their intrinsic microporosity.^{17,20} These particles are biologically inert and safe for use as biocompatible materials.

However, it is believed that use of synthetic eumelanin and allomelanin analogues as additives in cell-free biological reactions has not yet been explored. This example evaluates synthetic melanin for use as additives for protection against biologically damaging UV exposure, for example, for cell-free biological reactions.

E. coli-based cell-free expression systems have been applied for point-of-use technologies such as antibody and peptide production as well as for rapid biosensors for environmental water quality monitoring. These systems are compatible with freeze-drying, and previous studies have assessed additives for lyoprotection and long-term storage.^{21,23-25} This offers an opportunity to expand cell-free additives for the prevention of biological damage caused by UV exposure events, which has not yet been characterized for E. coli cell free expression systems. In previous work, these reactions were packaged to reduce or remove exposure to light. While the effects of UV exposure are not well-studied for cell-free systems, UV has damaging effects on biological systems. UV radiation can be divided into three types by their wavelength ranges: UVC (200-280 nm), UVB (280-320 nm), and UVA (320-400 nm). The highest-energy UVC radiation may be used for germicidal sterilization. UV irradiation generates lesions in DNA through photochemical reactions with dipyrimidine sites, with UVC being 100-fold more efficient to generate these lesions than UVB and UVB being around 1000-fold more efficient than UVA. Natural sunlight is primarily made up of 95% UVA and 5% UVB as most of the UVC and UVB radiation are removed by the earth's ozone layer. The literature suggests that an hour of sunlight can generate an estimated 520 lesions per 106 normal bases per J/cm² of UVB exposure in human skin.²⁹ Furthermore, UV irradiation also generates reactive oxygen species (ROS) that can rapidly react with biomolecules and lead to negative physiological effects (e.g., production of undesirable side-products or induces mutations in DNA³⁰). In general, biological systems are particularly vulnerable to ionizing radiation due to the presence of water which ionizes readily to form ROS, accounting for approximately two-thirds of the biological damage produced by X-rays.³¹

Here, it is shown that using biologically compatible synthetic melanin nanoparticles may help protect cell-free reactions from environmental exposure, including UV exposure. To explore the use of these nanoparticles, it was evaluated: how introducing a nanoparticle as an additive to a cell-free reaction affected the biological activity of the reaction; how light exposure events affected reaction efficiency with or without the additive; and whether the reactions having the additive demonstrated compatibility and light protection with lyophilization protocols, which allows for point-of-use applications. This may be important for point-of-use applications where solar exposure may be high, may allow for reactions to be kept in the sun for reaction incubation, and may additionally be adapted for new applications where it is desirable to use UV light while retaining biocompatibility. Furthermore, success in protection against UV-irradiation may potentially translate to protection of cell-free systems against other types of irradiation sources such as gamma and x-rays.

### Example 2: Synthesis and Characterization of Synthetic Melanin Nanoparticles and Compatibility with Cell-Free Reactions

### Example 2A: Synthesis and Characterization

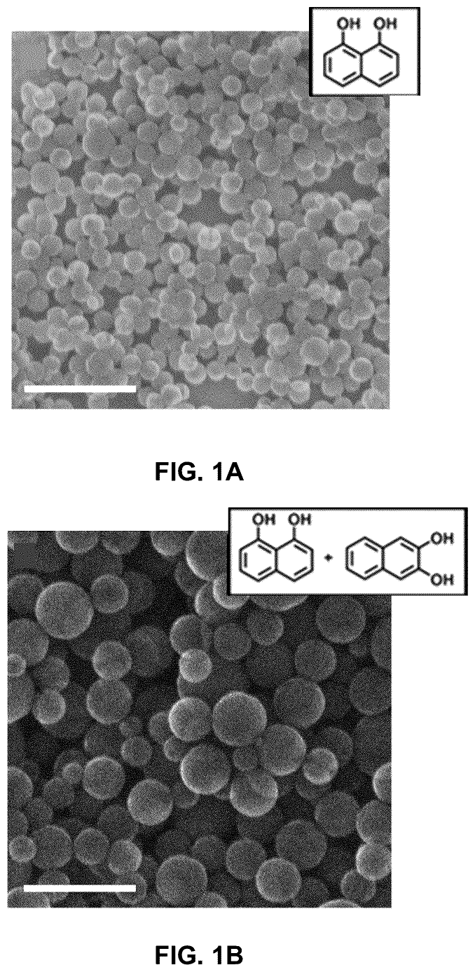

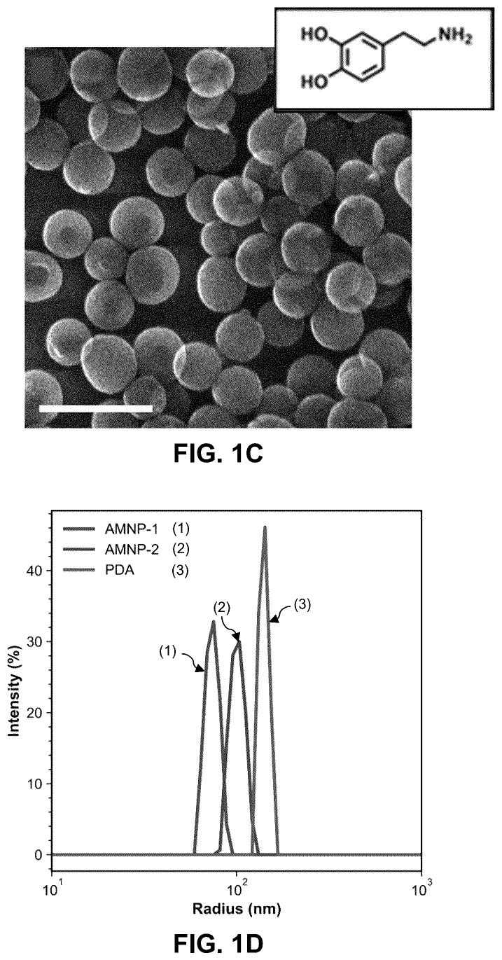

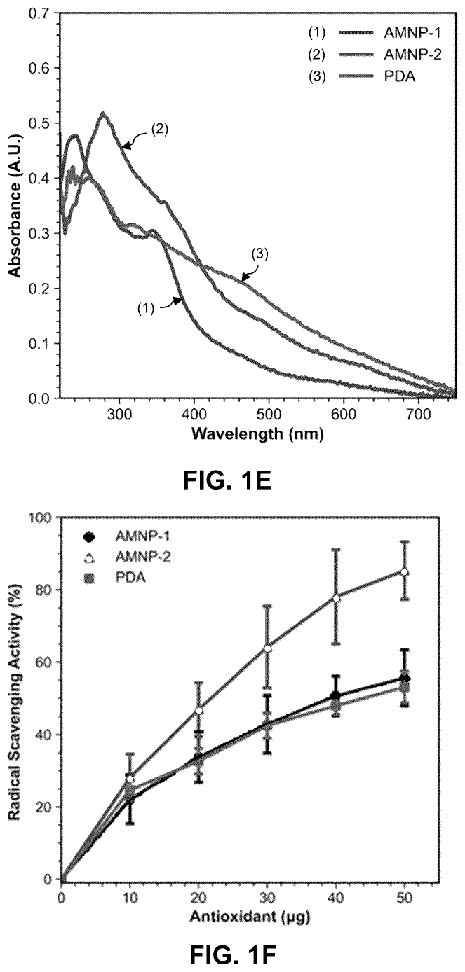

Synthetic eumelanin was prepared as described in Biyashev, D. et al., (npj Regener. Med. 2023, 8 (1), No. 61)³², via oxidative polymerization of dopamine with sodium hydroxide to yield polydopamine (PDA) nanoparticles. Two variations of synthetic allomelanin nanoparticles were prepared. The first variation of allomelanin nanoparticles (AMNP-1) was prepared as described in Zhou, X. et al., (ACS Nano 2019, 13 (1), 10980-10990)²⁰, via polymerization of 1,8 DHN monomer with sodium periodate oxidant. The second variation of allomelanin nanoparticles (AMNP-2) was prepared via the co-polymerization of 1,8 DHN and 2,3 DHN monomer in a 1:1 molar ratio. The rationale for inclusion of the 2,3 DHN monomer was that incorporation of the catechol groups would contribute toward the enhancement of radical scavenging activity. Synthesis of PDA, AMNP-1, and AMNP-2 yielded well-defined, colloidally stable spherical nanoparticles as determined by SE-STEM, dynamic light scattering (DLS), and zeta potential (see, FIGS. 1A-1D, and Table 1, below). Furthermore, each of the resulting synthetic melanin nanoparticles, AMNP-1, AMNP-2, and PDA, showed characteristic broadband absorption in their UV-vis absorption spectra, including for wavelengths between 200 nm and 400 nm (FIG. 1E). Another hallmark property of melanin is its antioxidant properties, which were assessed with a 2,2′-azinobis(3-ethylbenzothiazoline-6-sulfonic acid) (ABTS) radical scavenging assay across a range of melanin nanoparticle concentrations from 0 μg to 50 μg (recited as “Antioxidant (μg)” in FIG. 1F). As depicted in FIG. 1F, AMNP-2 exhibited the highest radical scavenging activity relative to AMNP-1 and PDA. For example, (i) at 20 μg: AMNP-2 demonstrated about 35%-55% radical scavenging activity, and each of AMNP-1 and PDA demonstrated about 25%-40%, and (ii) at 50 μg: AMNP-2 demonstrated greater than about 70% (e.g., 70%-95%) radical scavenging activity, and each of AMNP-1 and PDA demonstrated about 45%-70%.

TABLE 1
Characterization of Synthetic Melanin Nanoparticles.

| | Diameter (nm) | Zeta Potential (mV) |
|---|---|---|
| AMNP1 | 148 ± 13 | −30 ± 3 |
| AMNP2 | 204 ± 18 | −30 ± 4 |
| PDA | 254 ± 25 | −25 ± 0.6 |

### Example 2B: Compatibility of Synthetic Melanin Nanoparticles with Cell-Free Transcription and Translation

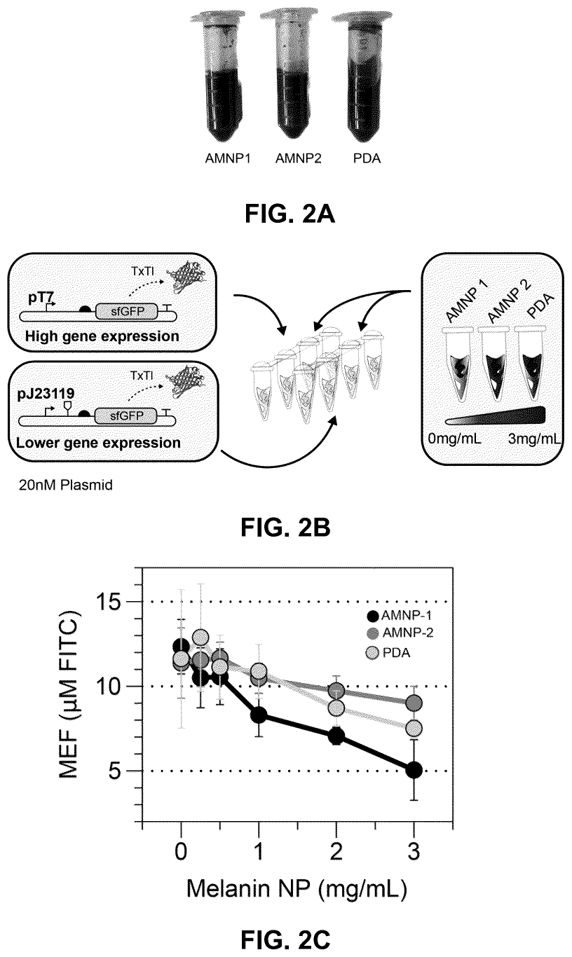

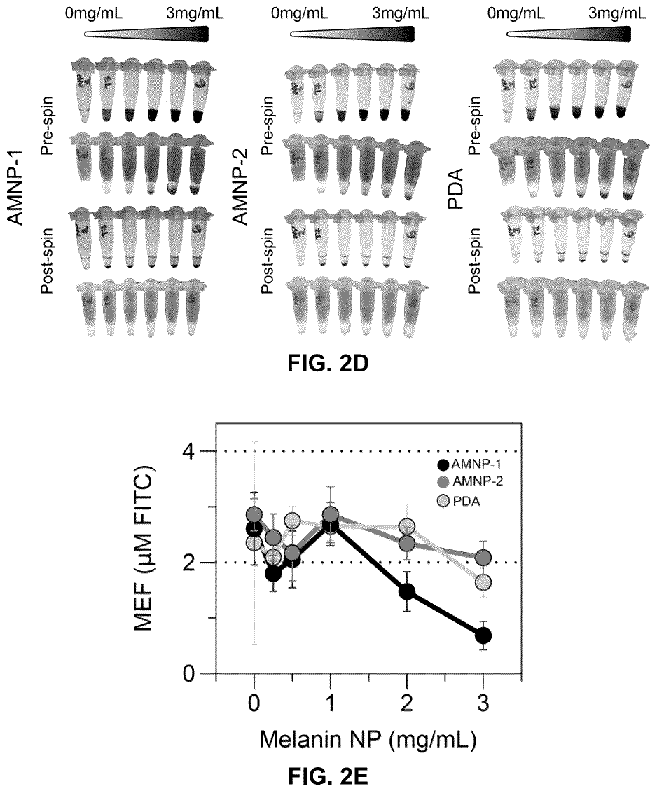

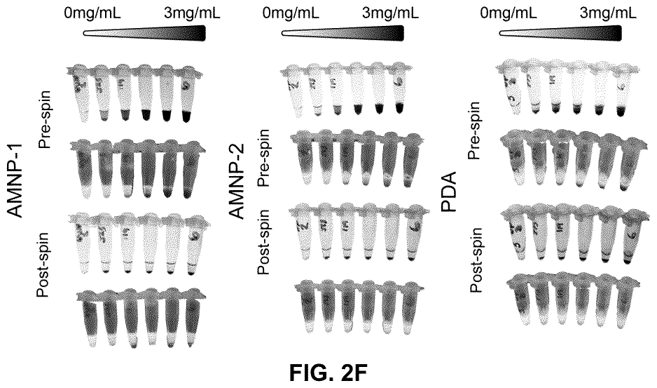

The nanoparticles synthesized in Example 2A were dark in coloration when prepared in 10 mg/mL solutions (see, FIG. 2A). To determine the compatibility of these materials with cell-free reactions, melanin nanoparticles were titrated into E. coli lysate-based cell-free reactions at concentrations of 0.25, 0.5, 1, 2, and 3 mg/mL (FIG. 2B). Super folder GFP (sfGFP) production was then assessed post-incubation for 20 h with differing concentrations of the nanoparticles under a strong T7 RNAP promoter (pT7) and weaker E. coli J23119 RNAP promoter (pJ23119). At low concentrations (0.25, 0.5 mg/mL) the reactions were minimally affected by the introduction of nanoparticles (FIG. 2C and FIG. 2E). AMNP-1 shows the most toxicity to cell-free reactions at higher concentrations for both promoter types. Expression plasmids with pT7 still retained highly visualizable fluorescence protein production even at high concentrations of melanin nanoparticles (FIG. 2C). For instance, at 1 mg/mL nanoparticles, the relative pT7-sfGFP production in the presence of AMNP-1, AMNP-2, and PDA was 8.3±1.3, 10.5±0.9, and 10.9±1.6 mean equivalent fluorescence (MEF) (μM fluorescein isothiocyanate (FITC)), respectively. Due to the lower overall activity of pJ23119-driven expression in comparison to pT7, higher concentrations of nanoparticles reduced normalized fluorescence values to low ranges (under ~2 MEF) (see, FIG. 2E). However, these reactions still produce a visualizable signal even at 3 mg/mL (FIG. 2E). Fluorescence was difficult to visually interpret in the presence of higher concentrations of nanoparticles, and reactions appear darker in color under normal lighting conditions as the concentration was increased (FIG. 2D and FIG. 2F). Overall, these experiments showed that the synthetic melanin nanoparticles were compatible with the transcriptional and translational machinery contained within the studied cell-free reactions.

### Example 3: Protective Properties of Melanin Nanoparticles and Other Materials

### Example 3A: Protective Properties of Melanin Nanoparticles

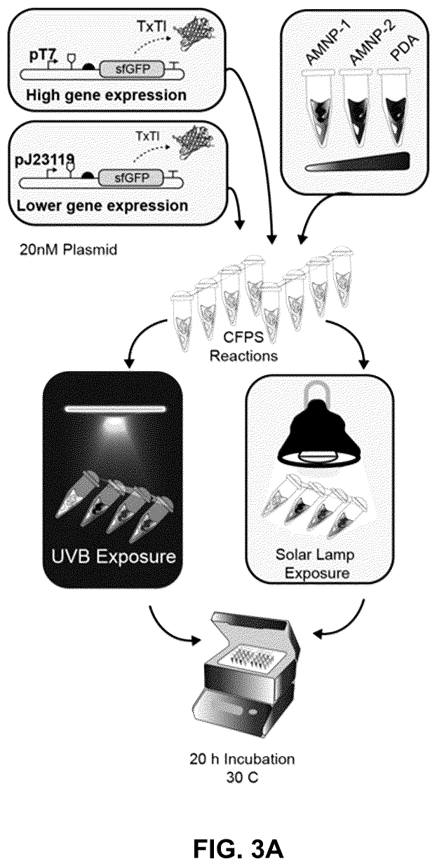

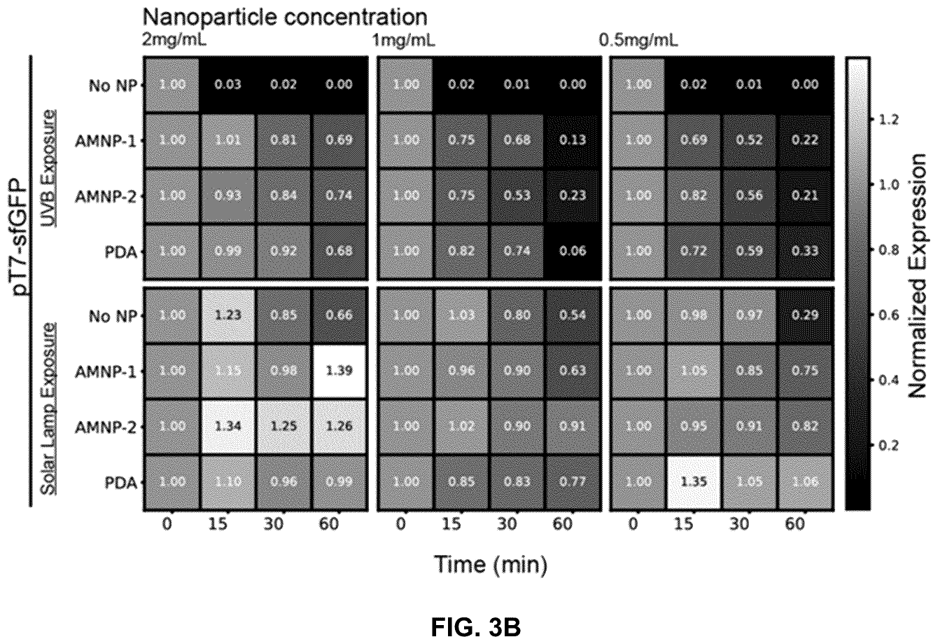

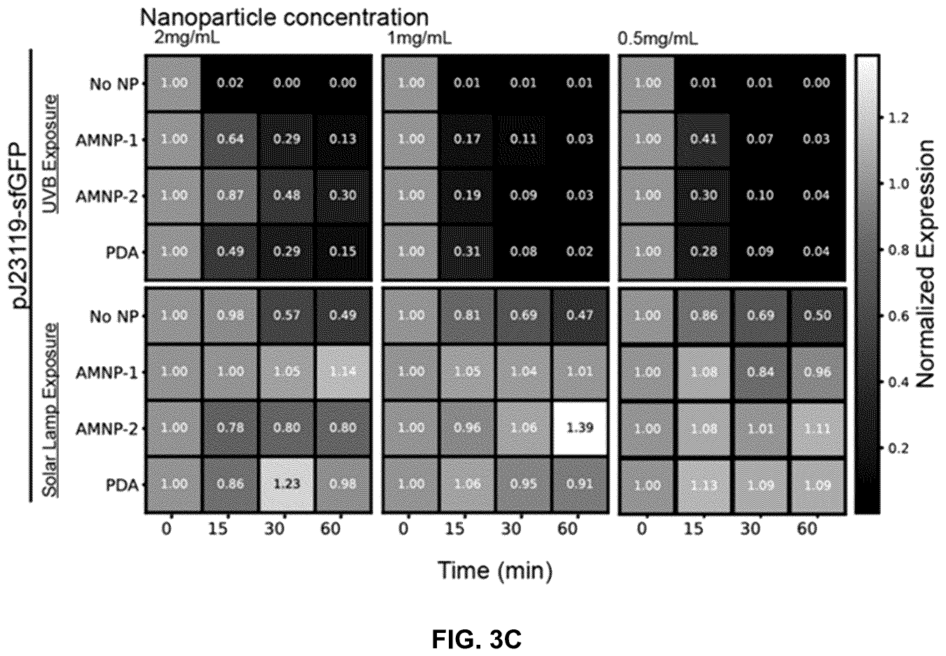

Next, we sought to characterize how light exposure affects cell-free gene expression and protection from exposure events in the presence and absence of the nanoadditives, AMNP-1, AMNP-2, and PDA. 20 μL cell-free reactions were constructed with pT7 or pJ23119 sfGFP expression plasmids and melanin additives at 0.5 mg/mL, 1 mg/mL, and 2 mg/mL and compared to that of with no additives (FIG. 3A). These reactions were then exposed to UVB or solar lamp light sources for 0, 15, 30, and 60 min on a cooling plate set to 4° C. Separate reactions were used for each of the time intervals so that each condition had continuous exposure for the given time condition. Reactions were then incubated at 30° C. for 20 h and characterized for sfGFP fluorescence (FIG. 3B and FIG. 3C).

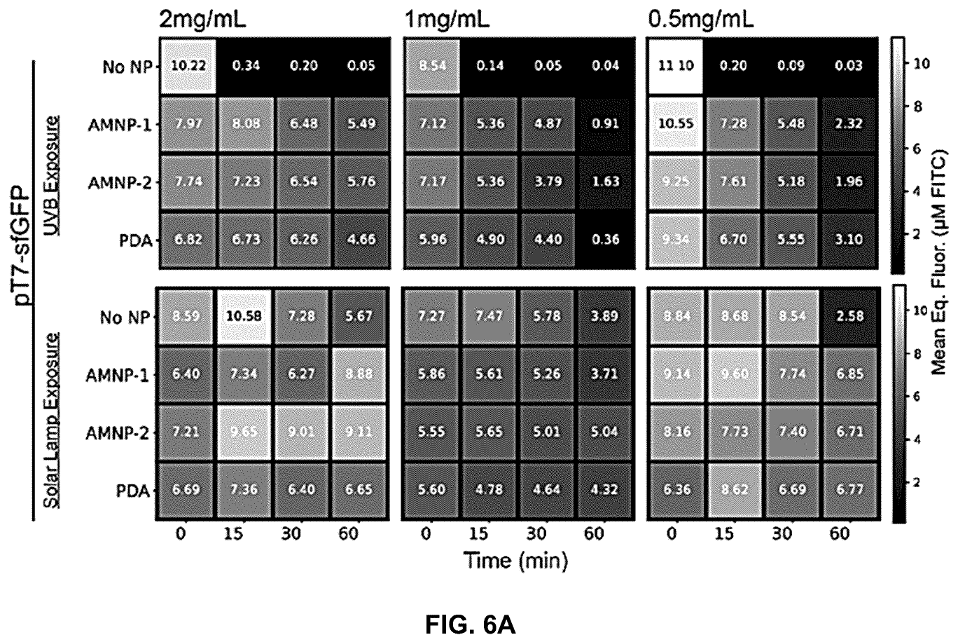

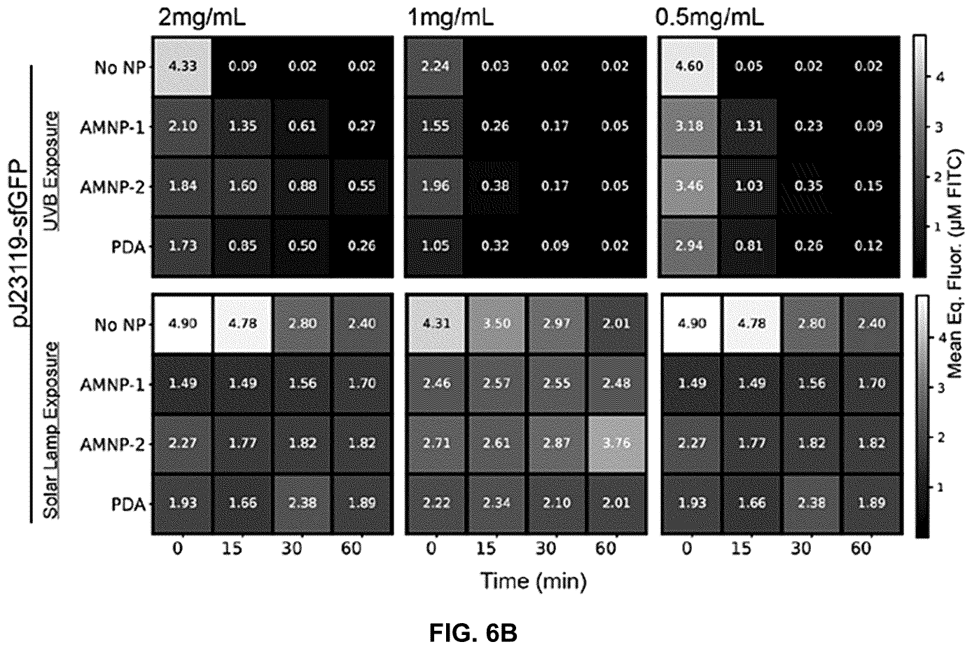

In both exposure settings, the addition of nanoparticles provided increased protection over an extended period in pT7 and pJ23119-sfGFP expression (FIGS. 3B and 3C), with normalized protection significantly better for the pT7 construct, especially for UVB exposure. It is noted that the T7 promoter is stronger than the J23119 promoter. This leads to pT7 reactions that retain a higher percentage of expression over the exposure time compared to pJ23119 reactions, indicating that while there are protective effects of the nanoparticles, the increased expression from pT7 constructs is likely the largest contributor to this retained expression over the exposure periods. As with pJ23119-sfGFP expression (depicted in FIG. 3C and FIG. 6B), the addition of 2 mg/mL nanoparticles to the pT7 reactions provided extended protective effects at the loss of overall expression (FIG. 3B and FIG. 6A). pT7 reactions exposed to UVB light retained greater than about 50% expression up to 30 min across all nanoparticle concentrations (FIG. 3B and FIG. 6A). For solar exposure, the reactions were well protected across all concentrations of nanoparticles, though solar lamp exposure causes less reaction degradation without nanoparticles due to the lower intensity exposure of UV radiation, particularly within the UVB region.

Solar lamp exposure had weaker effects on pJ23119 reactions than when exposed to UV, which showed almost complete reduction of expression to about 1% levels compared to unexposed signal after only 15 min of exposure (FIG. 3C and FIG. 6B). In both tested exposure settings, the addition of synthetic melanin nanoparticles provided increased protection from 15 to 60 min exposure periods compared with reactions not containing particles (FIG. 3C and FIG. 6B). For UVB exposure, the addition of 2 mg/mL nanoparticle concentrations caused reactions to retain 29-48% of unexposed sfGFP expression at 30 min compared to lower concentrations, where sfGFP expression at 30 min was reduced to around 10% of the unexposed signal. While protection was improved in reactions containing 2 mg/mL nanoparticles, this concentration reduced the overall activity of the reactions without light exposure compared to reactions containing no melanin nanoparticles (FIG. 6B). For solar lamp exposures, 2, 1, and 0.5 mg/mL nanoparticle additions across all exposure times (0, 15, 30, and 60 minutes) showed that reactions showed at most ~20% signal loss compared to controls.

### Example 3B: Synthetic Melanin Nanoparticles are More Biocompatible and Effective than Common UV Protectants

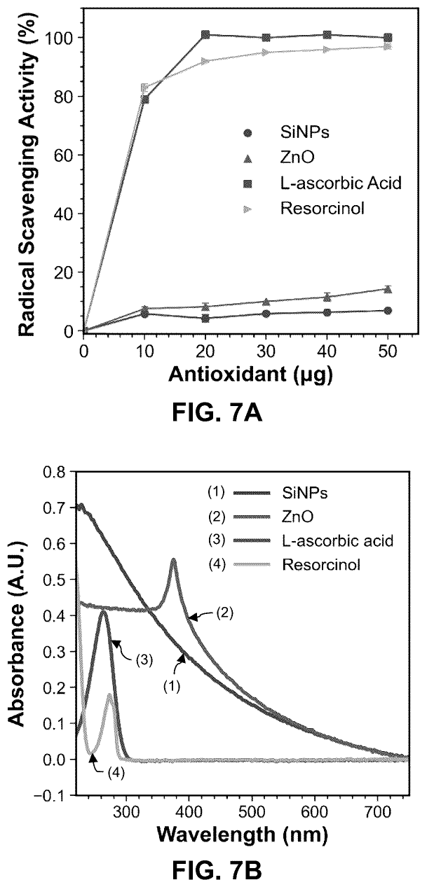

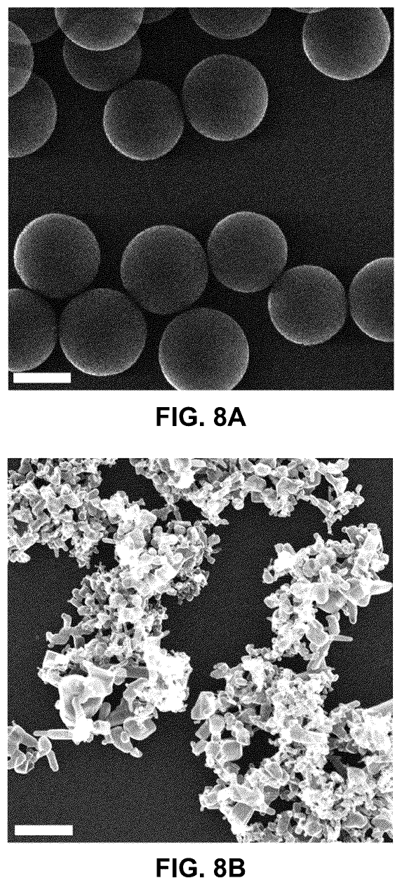

While synthetic melanins are excellent radical scavengers and broadband absorbers, there are alternative materials that could be used to protect against UV-radiation. We tested a variety of protective materials such as zinc oxide (ZnO), silica nanoparticles (SiNPs), L-ascorbic acid, and resorcinol, which all have different UV-protective properties. Carbon black exhibits broadband absorption analogous to melanin but is limited by low solubility in aqueous solutions, making it incompatible with cell-free systems. L-ascorbic acid and resorcinol were chosen as strong radical scavengers (see FIG. 7A and FIG. 7B). Metal oxide nanomaterials such as silica nanoparticles (SiNP) and zinc oxide (ZnO) have low radical scavenging activity but can also protect against UV-radiation through scattering effects³³ (see FIGS. 7A, 7B, 8A, and 8B).

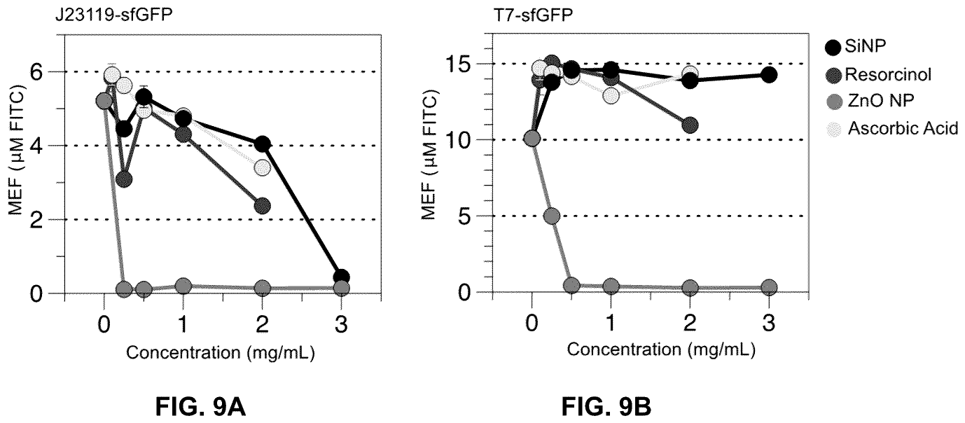

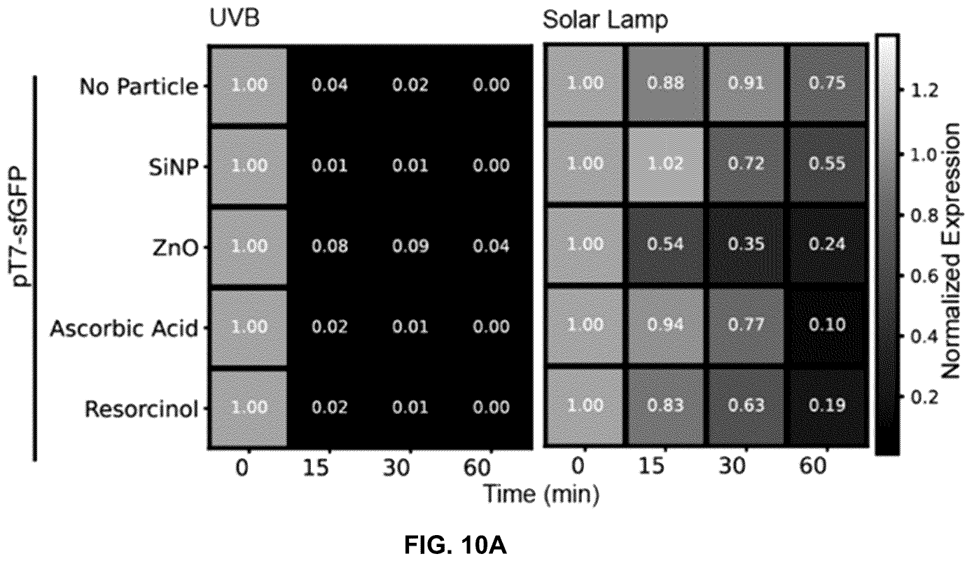

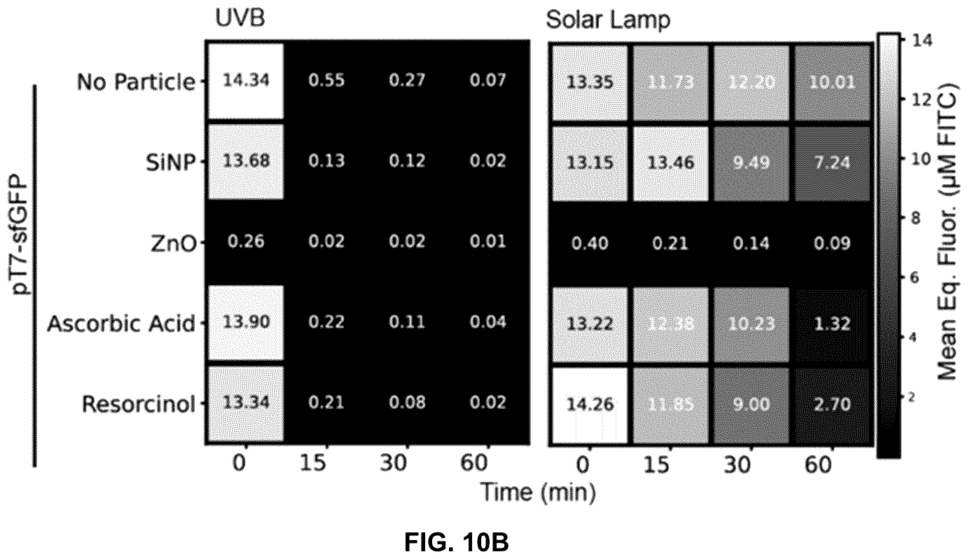

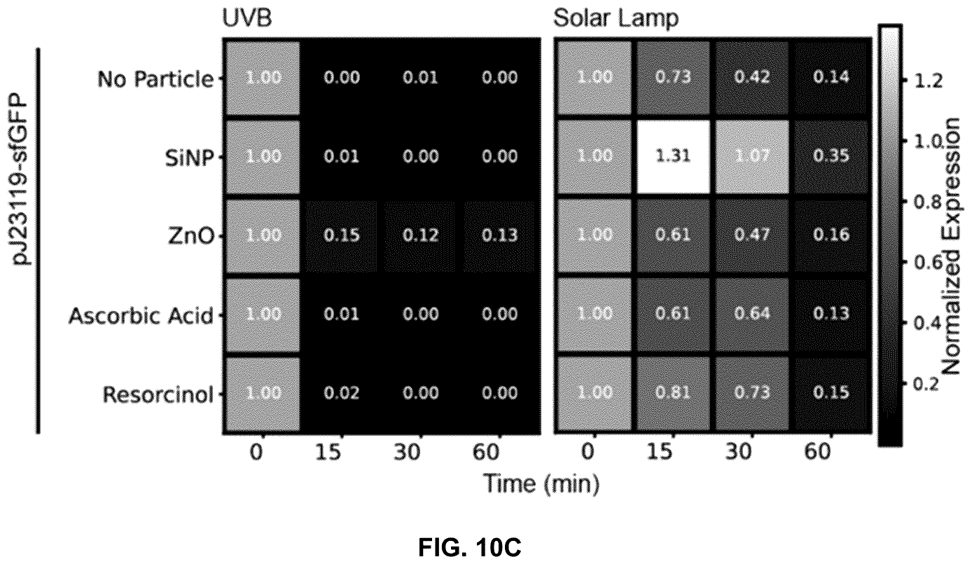

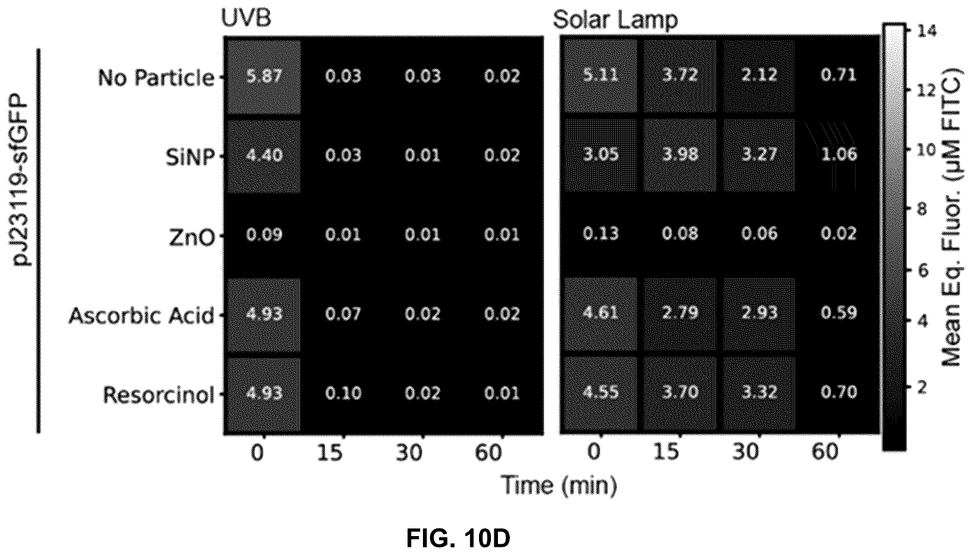

To determine whether alternative materials could also protect cell-free systems from UV-radiation and draw insight into the protection mechanism of cell-free systems against light irradiation, the same irradiation experiments described in Example 3A, above, were carried out with the addition SiNP, L-ascorbic acid, ZnO, and resorcinol. All the alternative radiation protectant additives showed reaction compatibility comparable to synthetic melanin nanoparticles except for the ZnO nanopowder, which significantly reduced sfGFP production at all observed concentrations for both pJ23119 (FIG. 9A) and pT7 (FIG. 9B) sfGFP expression. After initial compatibility tests, UVB and solar irradiation was applied on the cell-free systems with these alternative UV-protective additives, and resulting pJ23119 or pT7 sfGFP expression was measured. All the studied alternative radiation protectant additives were not sufficient to protect the pT7 (FIGS. 10A and 10B) and pJ23119 (FIGS. 10C and 10D) sfGFP expression against the UVB lamp and solar irradiation thus further demonstrating the benefits of using the synthetic melanin nanoparticles as opposed to other additives.

### Example 3C: Relationship Between Synthetic Melanin Nanoparticle Protection and Gene Expression Levels

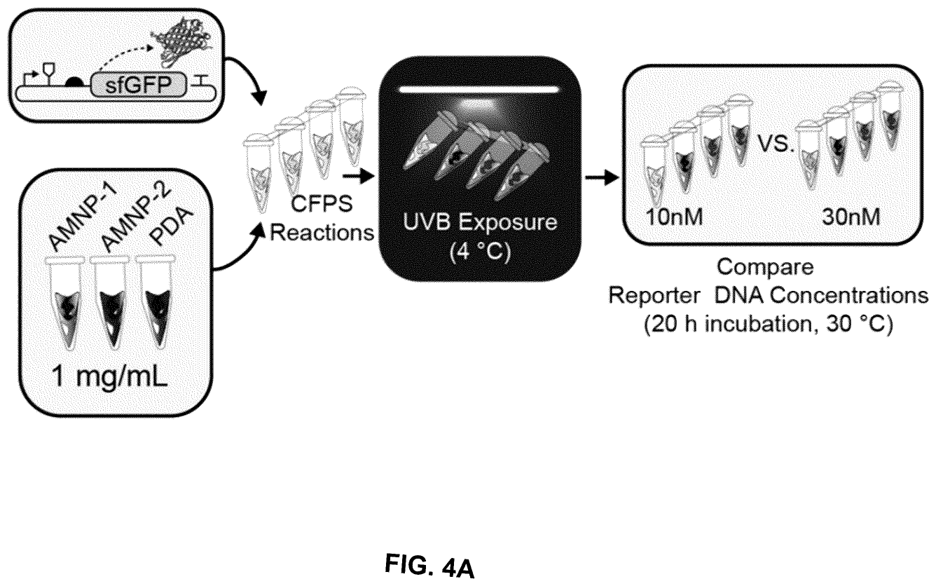

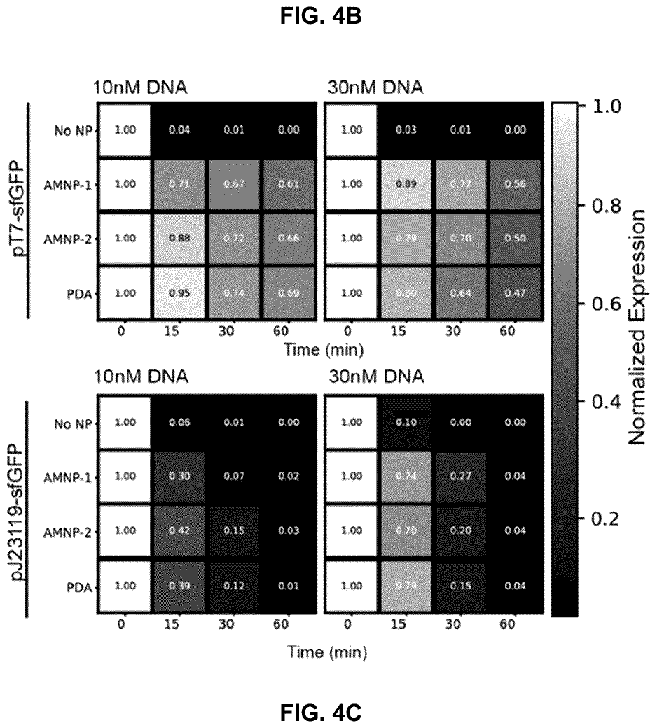

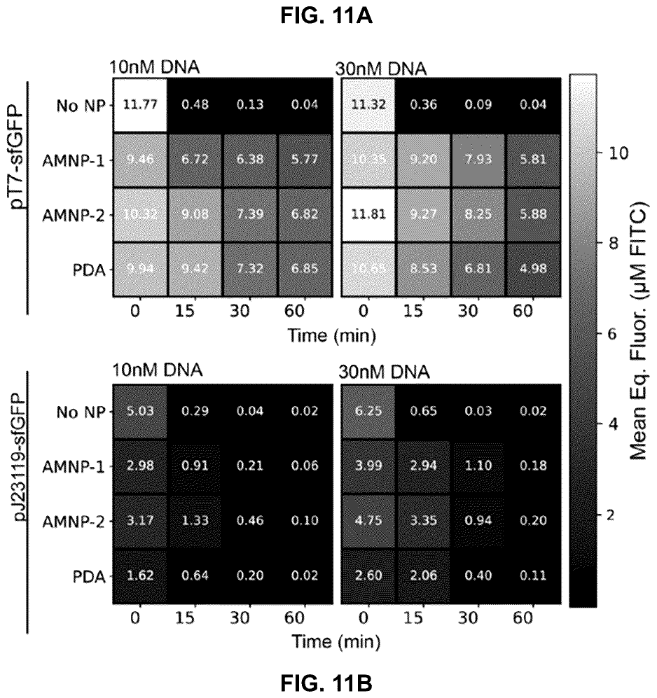

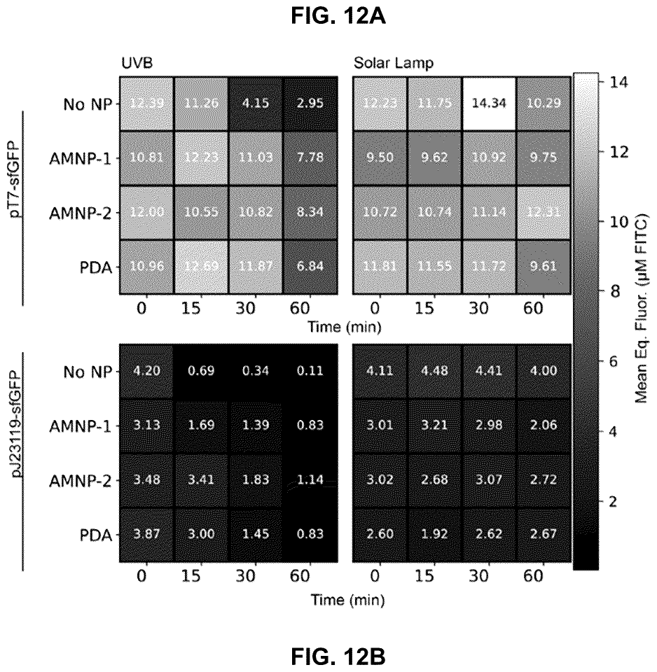

To further assess if changes in reaction conditions would lead to different outcomes on the protective effects of the synthetic melanin nanoparticles, we compared lower and higher sfGFP expressing DNA concentrations, 10 and 30 nM, respectively, at 1 mg/mL of nanoparticles, under exposure conditions (FIG. 4A). For pT7 expression, no clear improvement with the addition of higher DNA concentrations was observed (FIG. 4B), as 10 nM and 30 nM DNA concentrations had similar endpoint fluorescence values in zero exposure conditions (FIG. 4B and FIG. 11A, respectively). For pJ23119 expression, increasing the concentration of DNA resulted in more extended UVB resistance, where 30 nM DNA was able to retain 70-79% of unexposed signal after 15 min compared to 10 nM which was able to retain 30-42% of unexposed signal (FIG. 4C and FIG. 11B). This is likely due to higher gene expression for conditions containing 30 nM sfGFP plasmid that works in conjunction with the protective effects imparted by the synthetic melanin nanoparticles (FIG. 11B). Overall, for reactions containing DNA components with weaker gene expression, tuning the DNA concentration can allow for improved function in UV exposure environments with the addition of synthetic melanin nanoparticles.

### Example 3D: Cell-Free Reactions Comprising Synthetic Melanin Nanoparticles are Compatible with Lyophilization

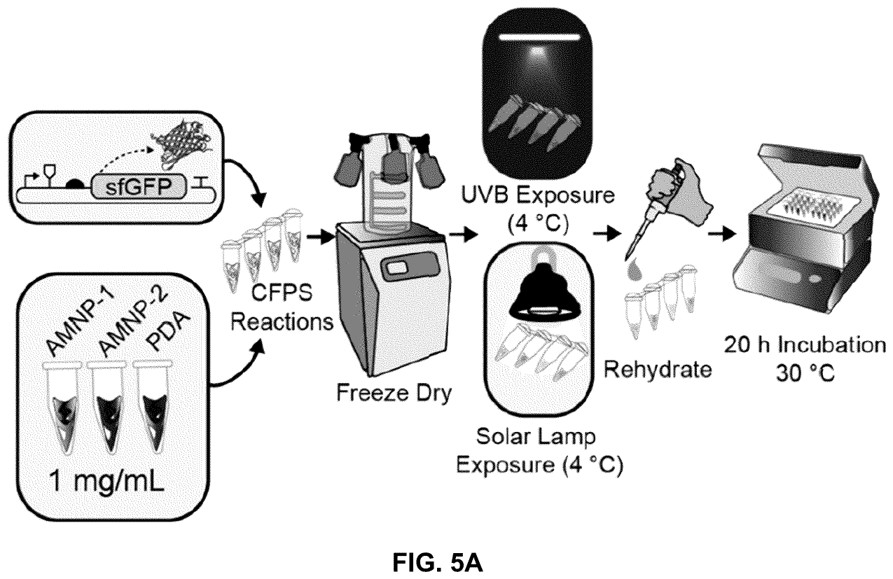

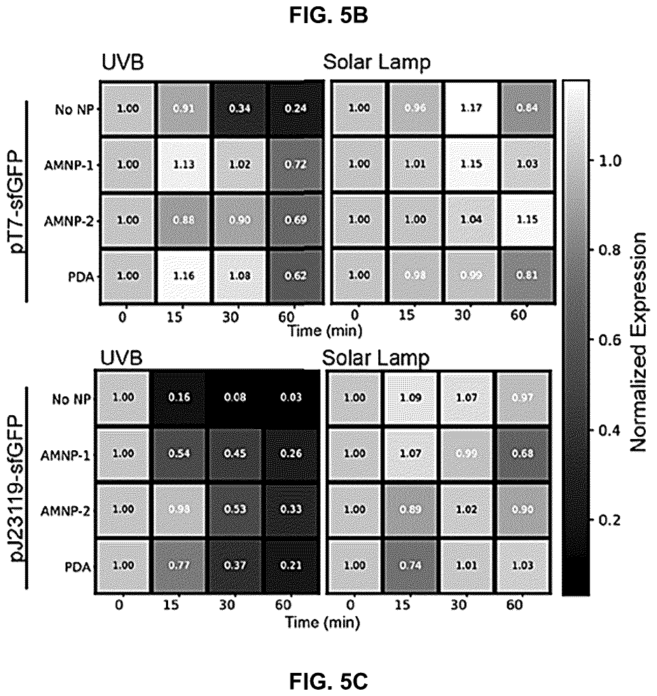

A benefit of cell-free reactions is that they can be freeze-dried and rehydrated at the point-of-need without use of cold chain shipment. Therefore, compatibility of synthetic eumelanin and allomelanin nanoparticles to freeze drying was assessed, as well as their protective capabilities in this state (FIG. 5A). Reactions containing 1 mg/mL synthetic melanin nanoparticle, 20 nM pJ23119-sfGFP or pT7-sfGFP expression plasmid, and cell-free reaction components were lyophilized overnight and then exposed to UVB or solar lamp radiation at 15-minute intervals. Under both promoters, lyophilization imparted a protective effect on the reactions without the presence of nanoparticles (FIG. 5B (pT7) and FIG. 5C (pJ23119)). For UVB exposure, nanoparticles were still able to impart a protective effect that worked in conjunction with the protective effects of freeze-drying. In these experiments, freeze-drying protected against solar lamp exposure for both promoters.

### Discussion Corresponding to Examples 1-3

Though there have been many studies about the effects of UV and light exposure on specific biochemical and biological systems, to the best of our knowledge there has been no characterization of exposure on E. coli cell-free reaction systems using a PANOx reaction system, which describes the use of phosphoenolpyruvate, amino acids, NAD+, and oxalic acid reaction reagents. Here, it was observed that solar and UV irradiation substantially affect the reaction performance. The reduced activity due to solar and UV exposure notably impacts point-of-use applications for cell-free manufacturing or sensing, two of the largest technological application areas of such systems.

Though it is often recommended to carry these reactions out in a closed environment, the use of melanin nanoparticles as additives can reduce the need for light protective packaging and allow users in point-of-need environments to carry these reactions out in the sun without substantially impacting reaction efficiency. It is also generally recommended to run reactions at the point-of-need on one's person, e.g., by placing reactions on bodily areas to provide enough heat to run the reaction. Here, reactions could be placed in the sun to heat them without significant loss of activity. This allows simpler use of reactions containing melanin nanoparticles, an important aspect of point-of-use technologies. It is known in the art that exposure limits, storage conditions, and other factors may impact the protective effect of an additive to a reaction mixture. However, it is hypothesized that the melanin nanoparticles discussed herein may result in protection beyond the 60 minute duration discussed in the examples herein, for example, ranges of days (e.g., 1-5 days, 1-30 days, etc.) are conceivable for sunlight or UV radiation exposure, depending on exposure limits and storage conditions.

In aspects, the use of the additives disclosed herein are valuable in agricultural or environmental sensing application areas, where it is often desirable to deploy biological/cell-free reactions in the environment to carry out beneficial functions. In aspects, the use of the additives disclosed herein are valuable for biological applications in space.

The examples discussed herein additionally further the application space for melanin-derived synthetic nanoparticles. In aspects, the synthetic melanin nanoparticles are used as a model for their protective capabilities, optionally wherein the model is broadly applied to retaining biological functions in different reaction conditions. Though these particles have shown the ability to scavenge oxygen released by UV reactions and UV-protective effects have been characterized, aspects discussed herein describe their low toxicity on biological systems and/or demonstrate their protective effects on cell-free reactions. Furthermore, other radiation protective additives including antioxidants (L-ascorbic acid and resorcinol) and high-Z light scatterers (SiNPs and ZnO) did not show the same level of protection against radiation as the synthetic melanin nanoparticles tested in Examples 1-3, indicating the utility of these specific materials as protective additives.

While each of the synthetic melanin nanoparticles used in Examples 1-3 demonstrated UV protection for cell-free systems, in some embodiments, it was difficult to draw direct comparisons between the performance of the different melanin nanoparticles. Even slightly different arrangements of the systems underneath the UV or solar lamps could lead to slightly different UV exposures, which would affect the relative expression. Even though AMNP-2 showed significantly higher levels of radical scavenging activity than AMNP-1 and PDA, all three synthetic melanin nanoparticles had similar trends of UV-protective capabilities within the cell-free systems. This further indicates that at these exposures, broadband absorption facilitated by the synthetic melanin nanoparticles may play a role in its UV-protective capabilities over radical scavenging activity. This is also hypothesized to explain why antioxidants like L-ascorbic acid and resorcinol had limited effects on protecting cell-free systems from radiation.

A potential limitation of the synthetic melanin nanoparticles generated in Examples 1-3 (e.g., AMNP-1 and AMNP-2) is that their coloration/pigmentation may make it difficult to use these nanoparticles for biosensor reactions or cell-free applications that are reliant on color changes, as opposed to a fluorescent or protein product output. However, some fluorescence can still be seen within reactions, even in the presence of the synthetic melanin nanoparticles, particularly at lower concentrations. Indeed, the reactions described above in Example 3B yielded observable MEF signals from a cell-free reaction (production of sfGFP) even in the presence of the synthetic eumelanin and allomelanin nanoparticles, particularly at lower concentrations (e.g., less than or equal to 3 mg/mL). For cell-free applications in protein production, proteins would need to be purified or processed from the reactions, with which the particles could be easily removed by centrifugation and other approaches.

In aspects, the radiation protection imparted by synthetic melanin nanoparticles is useful for application of cell-free protein synthesis in environments with high UV indices as well as potentially other extreme environments with ionizing radiation, including areas with intense solar irradiation, space/extraterrestrial, or nuclear facilities. Deployment where solar exposure may be high can allow for melanin nanoparticle-containing reactions to be kept in the sun for reaction incubation and can additionally be adapted for new applications, where it is desirable to use UV light while retaining biocompatibility. Furthermore, success in protection against UV irradiation may potentially translate to protection of cell-free systems against other types of irradiation sources such as γ and X-rays.

### Exemplary Experimental Aspects for Examples 1-3

Materials: 1,8-Dihydroxynapthalene (1,8-DHN) (95+%, Catalog No. 76152) was purchased from Matrix Scientific. 2,3-Dihydroxynapthalene (2,3-DHN) (98%, Catalog No. A11307) was purchased from Alfa Aesar. Dopamine hydrochloride (99%, Catalog No. A11136.22), 2,2-azino-bis-3-ethylbenzothiazoline-6-sulfonic acid (ABTS), sodium periodate (NaIO₄) (98.5%), sodium hydroxide (NaOH), potassium persulfate, high-performance liquid chromatography (HPLC) grade acetonitrile (ACN), and HPLC grade ethanol were purchased from Thermo Fisher Scientific. Zinc oxide (ZnO) nanopowder (<100 nm particle size), ammonium hydroxide (28-30%), and L-ascorbic acid were purchased from Sigma-Aldrich. Tetraethyl orthosilicate (TEOS) was purchased from Acros Organics. Grids for transmission electron microscopy (TEM) were purchased from Electron Microscopy Sciences (EMS).

Instrumentation: Scanning transmission electron microscopy (STEM) micrographs were taken with an SE-detector on Hitachi HD2300 at an accelerating voltage of 200 kV. Hydrodynamic radii were measured with a Wyatt Technology DynaPro NanoStar dynamic light scattering (DLS). Zeta potential of the nanoparticles were measured using Malvern Instruments Ltd, Nano Zetasizer in ultapure water at room temperature. UV-Vis spectra of the synthetic melanin nanoparticles, L-ascorbic acid, and resorcinol were measured at 0.01 mg/mL in ultrapure water and SiNPs and ZnO at 0.1 mg/mL in ultrapure water on a ThermoScientific Nanodrop 2000c spectrophotometer. ABTS colorimetric radical scavenging assay measurements were acquired using PerkinElmer Enspire Multimode Plate Reader.

Synthesis of AMNP-1. AMNP-1 was synthesized according to the procedure outlined in Zhou et al.²⁰ Briefly, about 150 mg of 1,8DHN was dissolved in about 7.5 mL of ACN and stirred for a few minutes before adding about 142.5 mL of ultrapure water. Then, this mixture was injected with a 1 mL solution of NaIO₄ (0.47 mmol, 100.15 mg) in ultrapure water and left to stir on a stir plate for about 20 hours. The particles were washed three times via centrifugation with ultrapure water (11000 rpm, 10 minutes).

Synthesis of AMNP-2. AMNP-2 was synthesized in a similar fashion to that of AMNP-1. Briefly, about 75 mg of 1,8DHN and about 75 mg of 2,3DHN was dissolved in about 7.5 mL of ACN and stirred for a few minutes before adding about 142.5 mL of ultrapure water. Then, this mixture was injected with a 1 mL solution of NaIO₄ (0.47 mmol, 100.15 mg) in ultrapure water and left to stir on a stir plate for about 20 hours. The particles were washed three times via centrifugation with ultrapure water (11000 rpm, 10 minutes).

Synthesis of PDA. The synthesis of PDA was based off a previously reported procedure described in Biyashev, D., et al.³² with modifications. Briefly, about 150 mg of dopamine-hydrochloride monomer was dissolved in about 100 mL of ultrapure water, injected with a solution of NaOH (1M, 0.750 mL) and polymerized for about 20 hours before being centrifuged and washed three times via centrifugation with ultrapure water (11000 rpm, 10 minutes).

Synthesis of Silica Nanoparticles. SiNPs were synthesized using the modified Stöber method based on a previously reported procedure in Zhou et al.²⁰ Briefly, silica seeds were synthesized by mixing tetraethyl orthosilicate (TEOS, 0.48 mL) with ethanol (8 mL) for about 10 minutes. This solution was subsequently injected with a solution composed of ammonium hydroxide (28-30%, 0.7 mL), ethanol (8 mL) and ultrapure water (1.2 mL) dropwise at room temperature for 6 hours before being washed three times via centrifugation (11000 rpm, 10 minutes). The silica seeds were lyophilized. The silica seeds (12.6 mg) were then dispersed in ultrapure water (0.6 mL), then mixed with ethanol (4 mL) and ammonium hydroxide (28-30%, 0.4 mL). Then a separate solution of TEOS (0.24 mL) and ethanol (4 mL) was prepared then added to the silica seed solution and stirred for about 85 minutes. The resulting SiNPs were then washed three times via centrifugation (11000 rpm, 10 minutes).

ABTS Radical Scavenging Assay. An 2,2′-azino-bis(3-ethylbenzothiazoline-6-sulfonic acid) diammonium salt (ABTS) radical scavenging activity of the melanin nanoparticles was based off a previously reported procedure in Noman et al.⁴² with modifications. Briefly, a bulk solution of potassium persulfate (2.45 mM) and ABTS (7 mM) was prepared and left in the dark for 16 hours. The ABTS solution was diluted such that the final stock solution was 0.2 mM ABTS. Melanin nanoparticles were used 2 months after synthesis. Two duplicate sets of melanin nanoparticle solutions constituting of 10, 20, 30, 40, and 50 μg of nanoparticles in 100 μL of ultrapure water were prepared. In one set, 1.5 mL of the diluted ABTS stock solution (0.2 mM) was added and left in the dark for about 20 minutes. After about 20 minutes, all solutions were read on a plate reader at 734 nm (A₁) To the other set of melanin nanoparticle solutions, 1.5 mL of water was added to each make “blank” solutions and its absorbance at 734 nm was measured (A₂). Finally, 1.5 mL of the diluted ABTS stock solution (0.2 mM) was added to 100 μL of water and its absorbance at 734 nm was measured (A₀) To calculate the radical scavenging activity at each concentration of nanoparticles added, the following equation was utilized:

$$A B T S ( \% ) = 1 - ( \frac{( {A}_{1} - {A}_{2} )}{{A}_{0}} ) \times 100 \%$$

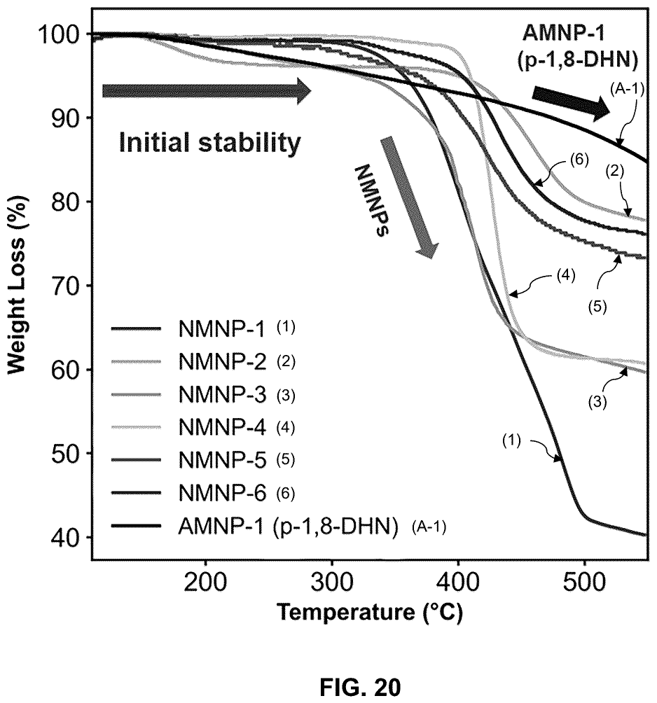

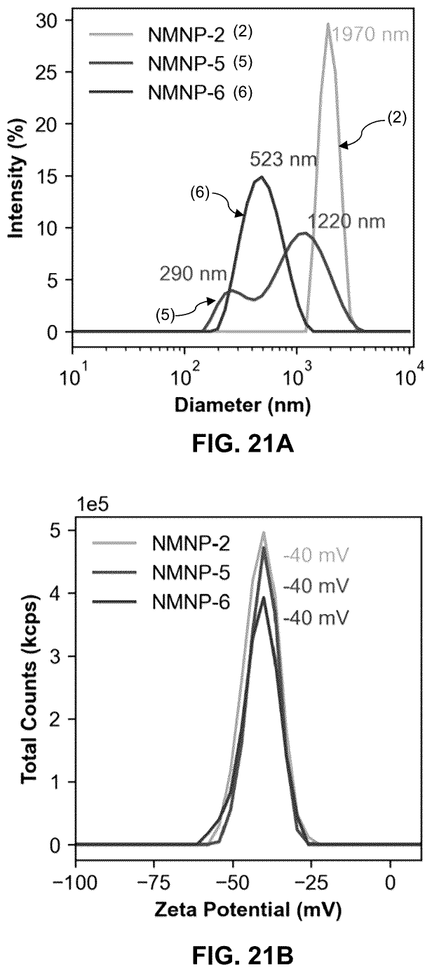

Cell Free Expression Reactions. E. coli lysate was prepared using BL21 (DE3)* with previously published protocols. Cell-free reactions were prepared using a PANOx reagent system. This system uses the following approximate amounts; salt solution containing 8 mM magnesium glutamate, 10 mM ammonium glutamate, 130 mM potassium glutamate; transcription master mix with 1.2 mM ATP; 0.850 mM GTP, UTP, and CTP, 72 μM folinic acid, 0.171 mg/mL tRNA; amino acids solution with 2 mM amino acids; energy solution of 30 mM PEP; and cofactor solution with 0.33 mM NAD, 0.27 mM CoA, 4 mM oxalic acid, 1 mM putrescine, 1.5 mM spermidine, 57 mM HEPES. This mixture accounts for about 30% of the reaction volume. Cell extract was also used at about 30% of the reaction volume. For each experiment, DNA and the melanin nanoparticles were diluted in the water component of the reaction to make up the additional reaction volume (about 40%). Except for FIGS. 4A-4C, 20 nM of pJ23119-sfGFP and pT7-sfGFP DNA was used in all experiments. All reactions were carried out at a total volume of about 20 μL and incubated at about 30° C. for about 20 h post light exposure. The reactions are then rehydrated and spun on a centrifuge for about 20 minutes at 3700 rpm to remove the particles from the reaction. The supernatant is then collected and processed on a plate reader set to excitation/emission 485/520 nm. For reactions that were lyophilized we used a Labconco FreeZone 2.5 Liter-84C Benchtop Freeze Dryer for about 24 hours before rehydrating the reactions, spinning them with a centrifuge, mixing, exposing them to UVB or solar lamp conditions, and then incubating for about an additional 20 hours.

Cell-Free Gene Expression Reactions. Cell-free gene expression reactions with and without melanin nanoparticles were prepared by assembling all components on ice at a total volume of 20 UL and incubated at 30° C. for 20 h post light exposure described in the UVB and Solar Lamp Exposure section. The reactions were then spun on a centrifuge for 20 min at 3700 rpm to remove the particles from the reaction. 15 μL of supernatant was then collected in separate PCR tubes to ensure minimal residual nanoparticles were plated. 5 μL of each replicate was then plated in a low volume 384-well plate (Corning CLS3842) and fluorescence was measured in a plate reader set to excitation/emission 485/520 nm and read from the bottom at gain 50 or 60 and ambient temperature.

Lyophilized Reactions. Lyophilized reactions were prepared on ice as above, snap-cooled in liquid N₂, and then lyophilized using a Labconco FreeZone 2.5 Liter-84C Benchtop Freeze-Dryer for 16 h. Reactions were then immediately exposed to light conditions in the “UVB and Solar Lamp Exposure” paragraph below. Reactions were then rehydrated with 20 μL of Milli-Q water, spun with a centrifuge, mixed with a pipet, and then incubated for an additional 20 h at 30° C.

Data was calibrated to mean equivalent fluorescence (MEF) using FITC calibration, and this FITC-calibrated data was used to calculate the normalized expression. Normalized expression is calculated as the end point GFP expression of the light exposure condition divided by the non-light-exposed (t=0 min) condition for no nanoparticle, AMNP-1, AMNP-2, and PDA-containing samples. This provides a fractional reduction in expression due to light exposure. All experiments were done in triplicate. Data was then analyzed in python (see the “Data Processing” paragraph).

UVB and Solar Lamp Exposure. In each of the experiments with UVB or solar lamp exposure, reactions were placed on a cooling plate set to 4° C. to reduce the effects of heating the reaction with light and prevent gene expression before incubation, which could lead to unintended photobleaching of the reporter protein. UV/AB exposure of all sources was measured with a Sper Scientific UV/AB Light Meter (850009). UVB lamp (Spectronics X-15B, 15 W, 312 nm) was used, and reactions were placed directly on the cooling plate with the lamp sitting approximately 2.5 cm above, providing an average UV/AB exposure between approximately 6000-7000 μW/cm², with some variability in measurement. A box was used to cover the exposure setup. For solar lamp exposure, a solar lamp (MIXJOY Reptile Heat Lamp, 160 W) was used with the lamp placed 30 cm above the reactions corresponding to approximately 2500-3500 μW/cm² UV/AB exposure and temperature of 31° C. (88° F.) due to radiative heat from the lamp. This solar simulating lamp features a brightness of 1200 lm and covers the full UVA, UVB, visible, and infrared spectrum. This solar lamp condition was chosen to best represent solar exposure on a hot, sunny day with a high UVB exposure of 265 W/cm² according to the manufacturer's description. For reference, in particularly hot climates, UVB exposure can reach up to 350-450 μW/cm². Notably, in nature, UVB exposure can vary greatly by location, weather, and time of day. Reactions for each time point were made separately, so that each condition represents continuous exposure. The reactions were then placed on the cooling plate starting with the 60 min time point reactions, and then after 30 min of time passing, the 30 min exposure condition was added; after an additional 15 min of time passing, the reactions to be exposed for 15 min were added and sat under the light condition for the remainder of the time. Reactions were then processed in accordance with the methods described in the “Cell-Free Gene Expression Reactions” of the above paragraph.

Data Processing. Python files used to generate figures from the data files are provided in the Brief Description of the Drawings herein. All heat maps are presented as fold change normalized to the no light exposure controls for each figure to make it easier to distinguish patterns in the data, raw plate reader values are presented similarly in the Brief Description of the Drawings herein.

Plasmids Used in This Study. pJL1 (Addgene #69496) was used for pT7 synthesis of sfGFP. pJBL7010 (Addgene #136942) was used for pJ23119 sfGFP expression.

### REFERENCES CORRESPONDING TO EXAMPLES 1-3

(1) Cao, W., et al., Unraveling the Structure and Function of Melanin through Synthesis. J. Am. Chem. Soc. 2021, 143 (7), 2622-2637.
(2) Guo, L., et al., Recent Advances and Progress on Melanin: From Source to Application. Int J Mol Sci 2023, 24 (5), 4360.
(3) Tran-Ly, A. N., et al., Microbial Production of Melanin and Its Various Applications. World J Microbiol Biotechnol 2020, 36 (11), 170.
(4) Mattoon, E. R.; Cordero, R. J. B.; Casadevall, A. Fungal Melanins and Applications in Healthcare, Bioremediation and Industry. J Fungi (Basel) 2021, 7 (6), 488.
(5) Fordjour, E., et al., Metabolic Engineering of Escherichia Coli BL21 (DE3) for de Novo Production of I-DOPA from d-Glucose. Microb Cell Fact 2019, 18 (1), 74.
(6) Surwase, S. N.; Jadhav, J. P. Bioconversion of L-Tyrosine to I-DOPA by a Novel Bacterium Bacillus Sp. JPJ. Amino Acids 2011, 41 (2), 495-506.
(7) Wei, T.; Cheng, B.-Y.; Liu, J.-Z. Genome Engineering Escherichia Coli for L-DOPA Overproduction from Glucose. Sci Rep 2016, 6 (1), 30080.
(8) Yoshida, H.; Tanaka, Y.; Nakayama, K. Production of 3,4-Dihydroxyphenyl-I-Alanine (I-DOPA) by Pseudomonas melanogenum. Agricultural and Biological Chemistry 1974, 38 (2), 455-462.
(9) Walker, K. T., et al., Self-Pigmenting Textiles Grown from Cellulose-Producing Bacteria with Engineered Tyrosinase Expression. Nat Biotechnol 2024, 1-10.
(10) El-Bialy, H. A., et al., Microbial Melanin Physiology under Stress Conditions and Gamma Radiation Protection Studies. Radiation Physics and Chemistry 2019, 162, 178-186.
(11) Corani, A., et al., Superior Photoprotective Motifs and Mechanisms in Eumelanins Uncovered. J. Am. Chem. Soc. 2014, 136 (33), 11626-11635.
(12) Dadachova, E., et al., Ionizing Radiation Changes the Electronic Properties of Melanin and Enhances the Growth of Melanized Fungi. PLOS ONE 2007, 2 (5), e457.
(13) Dadachova, E.; Casadevall, A. Melanin and Resistance to lonizing Radiation in Fungi. In Extremophiles Handbook; Horikoshi, K., Ed.; Springer Japan: Tokyo, 2011; pp 1147-1157.
(14) Galván, I.; Solano, F. Bird Integumentary Melanins: Biosynthesis, Forms, Function and Evolution. IJMS 2016, 17 (4), 520.
(15) Koike, S.; Yamasaki, K. Melanogenesis Connection with Innate Immunity and Toll-Like Receptors. IJMS 2020, 21 (24), 9769.
(16) Lichtenegger, H. C., et al., High Abrasion Resistance with Sparse Mineralization: Copper Biomineral in Worm Jaws. Science 2002, 298 (5592), 389-392.
(17) McCallum, N. C., et al., Allomelanin: A Biopolymer of Intrinsic Microporosity. J. Am. Chem. Soc. 2021, 143 (10), 4005-4016.
(18) Biyashev, D., et al., Topical Application of Synthetic Melanin Promotes Tissue Repair. npj Regen Med 2023, 8 (1), 61.
(19) Huang, Y., et al., Mimicking Melanosomes: Polydopamine Nanoparticles as Artificial Microparasols. ACS Cent. Sci. 2017, 3 (6), 564-569.
(20) Zhou, X., et al., Artificial Allomelanin Nanoparticles. ACS Nano 2019, 13 (10), 10980-10990.
(21) Warfel, K. F., et al., A Low-Cost, Thermostable, Cell-Free Protein Synthesis Platform for On-Demand Production of Conjugate Vaccines. ACS Synth. Biol. 2023, 12 (1), 95-107.
(22) DeWinter, M. A., et al., Point-of-Care Peptide Hormone Production Enabled by Cell-Free Protein Synthesis. ACS Synth. Biol. 2023, 12 (4), 1216-1226.
(23) Thavarajah, W., et al., Point-of-Use Detection of Environmental Fluoride via a Cell-Free Riboswitch-Based Biosensor. ACS Synth. Biol. 2020, 9 (1), 10-18.
(24) Gregorio, N. E., et al., Unlocking Applications of Cell-Free Biotechnology through Enhanced Shelf Life and Productivity of E. Coli Extracts. ACS Synth. Biol. 2020, 9 (4), 766-778.
(25) Thavarajah, W., et al., The Accuracy and Usability of Point-of-Use Fluoride Biosensors in Rural Kenya. npj Clean Water 2023, 6 (1), 1-8.
(26) Raeiszadeh, M.; Adeli, B. A Critical Review on Ultraviolet Disinfection Systems against COVID-19 Outbreak: Applicability, Validation, and Safety Considerations. ACS Photonics 2020, 7 (11), 2941-2951.
(27) Pereira, R. V., et al., Evaluation of the Effects of Ultraviolet Light on Bacterial Contaminants Inoculated into Whole Milk and Colostrum, and on Colostrum Immunoglobulin G. Journal of Dairy Science 2014, 97 (5), 2866-2875.
(28) Yemmireddy, V., et al., Effect of Ultraviolet Light Treatment on Microbiological Safety and Quality of Fresh Produce: An Overview. Front. Nutr. 2022, 9, 871243.
(29) Lee, J. W., et al., Deciphering UV-induced DNA Damage Responses to Prevent and Treat Skin Cancer. Photochem & Photobiology 2020, 96 (3), 478-499.
(30) De Jager, T. L.; Cockrell, A. E.; Du Plessis, S. S. Ultraviolet Light Induced Generation of Reactive Oxygen Species. In Ultraviolet Light in Human Health, Diseases and Environment; Ahmad, S. I., Ed.; Advances in Experimental Medicine and Biology; Springer International Publishing: Cham, 2017; Vol. 996, pp 15-23.
(31) Health Effects of Exposure to Low Levels of Ionizing Radiation: BEIR V; National Academies Press: Washington, D.C., 1990; p 1224.
(32) Biyashev, D., et al., Topical Application of Synthetic Melanin Promotes Tissue Repair. npj Regen Med 2023, 8 (1), 61.
(33) Nambiar, S.; Yeow, J. T. W. Polymer-Composite Materials for Radiation Protection. ACS Appl. Mater. Interfaces 2012, 4 (11), 5717-5726.
(34) Silverman, A. D., et al., Deconstructing Cell-Free Extract Preparation for in Vitro Activation of Transcriptional Genetic Circuitry. ACS Synth. Biol. 2019, 8 (2), 403-414.
(35) Oppezzo, O. J. Contribution of UVB Radiation to Bacterial Inactivation by Natural Sunlight. Journal of Photochemistry and Photobiology B: Biology 2012, 115, 58-62.
(36) Santos, A. L., et al., Contribution of Reactive Oxygen Species to UV-B-Induced Damage in Bacteria. Journal of Photochemistry and Photobiology B: Biology 2012, 117, 40-46.
(37) Cacciari, R. D., et al., Effect of UVB Solar Irradiation on Laccase Enzyme: Evaluation of the Photooxidation Process and Its Impact over the Enzymatic Activity for Pollutants Bioremediation. Amino Acids 2020, 52 (6), 925-939.
(38) McMillan, T. J., et al., Cellular Effects of Long Wavelength UV Light (UVA) in Mammalian Cells. Journal of Pharmacy and Pharmacology 2008, 60 (8), 969-976.
(39) Kulms, D., et al., Nuclear and Cell Membrane Effects Contribute Independently to the Induction of Apoptosis in Human Cells Exposed to UVB Radiation. Proceedings of the National Academy of Sciences 1999, 96 (14), 7974-7979.
(40) Garmyn, M.; Yarosh, D. B. The Molecular and Genetic Effects of Ultraviolet Radiation Exposure on Skin Cells. In Photodermatology; CRC Press, 2007.
(41) Kocalar, S., et al., Validation of Cell-Free Protein Synthesis Aboard the International Space Station. ACS Synth. Biol. 2024, 13 (3), 942-950.
(42) Noman, A., et al., Characterization of Physicochemical Properties of Melanin Produced by Gluconobacter Oxydans FBFS 97. Fermentation 2022, 8 (11), 574.

### Example 4: Engineering Pigmentation of Allomelanin-Like Nanomaterials for the UV Protection of Cell-Free Systems

Melanins are a class of biological polymers that are found ubiquitously in nature, from plants and fungi to humans. The word melanin originates from the Green work “melanos,” which translates to black or very dark, which is reflected it its black/brown pigmentation.¹ Melanin can be classified into five different classes that are defined by their monomer precursors: eumelanin, pheomelanin, pyomelanin, neuromelanin, and allomelanin.² Eumelanin, which is polymerized from levodopa (L-DOPA), is representative of melanin found in animals such as in bird feathers and human hair and skin. Allomelanin is another important nitrogen-free melanin polymerized from a 1,8-dihydroxynapthalene (1,8-DHN) monomer precursor that is commonly found in plants and fungi.

As noted in above (e.g., Example 1), melanin exhibits several important physicochemical properties in addition to its characteristic black/brown pigmentation. One of its properties is its broadband absorption of light, and in particular UV-light, which enables melanin to protect organisms from harmful UV radiation from the sun or other sources. In a similar vein, melanin has also been shown to protect organisms from ionizing radiation through its radical scavenging activity. Harmful and highly reactive free radicals generated from incidents of ionizing radiation can be effectively quenched by melanin through interaction with its hydroxyl groups and conjugated network. These two properties in tandem are what make melanin a useful radiation protectant for organisms.

E. coli-based cell-free expression systems have been applied in point-of-use technologies, and such systems are attractive because of their transportability and long-term stability enabled by addition of specific additives and their lyocompatiblity.^{3,5-7} These point-of-use technologies are often in employed in rural areas or non-ideal climates, which may impact the effectiveness to deploy these cell-free systems. In Examples 1-3, above, the detrimental effect that UV exposure, including simulated solar radiation, had on the E. coli-based cell-free expression of sfGFP was demonstrated. In that work, it was also demonstrated that synthetic melanin nanoparticle additives could mitigate the harmful effects of UV radiation.

A potential limitation of synthetic melanin nanoparticles for generalized use in the protection of cell-free systems is its dark black/brown pigmentation that limits its utilization in cell-free biosensors, which requires observation of a real-time optical change to be functional. Having dark synthetic melanin nanoparticles suspended in the cell-free solution may hinder users from being able to observe that optical change to indicate whether the system is “on” or “off”. While the synthetic melanin nanoparticles can be removed via centrifugation or filtration to observe this optical change, this adds undesirable steps to biosensor handling, which can lead to more human error and failure of the systems.

In this Example, it is shown that the pigmentation of allomelanin-inspired nanomaterials through the strategic selection and polymerization of dihydroxynapthalene isomers can be engineered. These melanin-inspired nanomaterials were shown to be compatible with E. coli-based cell-free systems and protected them against UVB and solar radiation. Furthermore, by engineering the pigmentation of these nanomaterials, these also provided UV protection of E. coli-based cell-free sensors while still enabling device-based visualization of the sensor.

### Example 4.1 Synthesis and Characterization of Neo-Melanin Nanoparticle Library

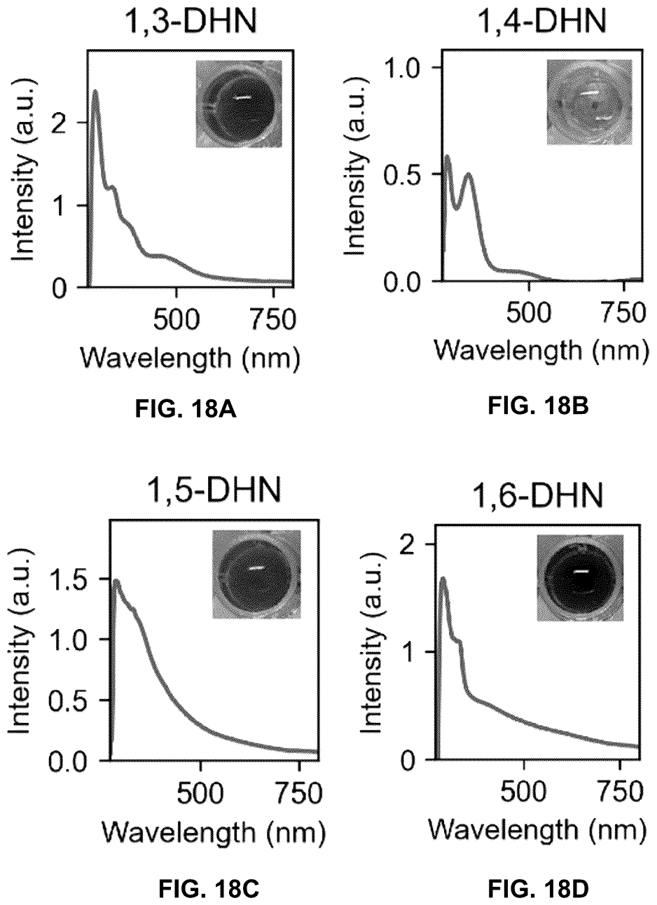

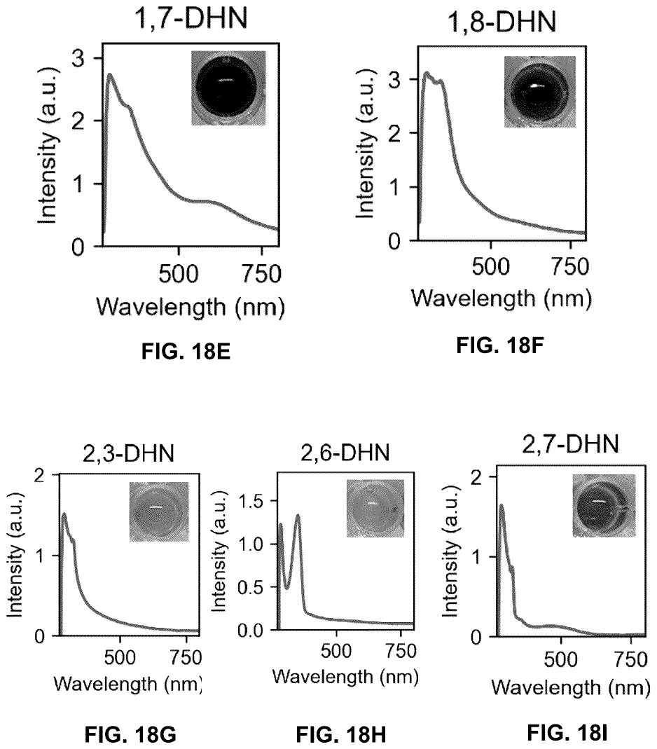

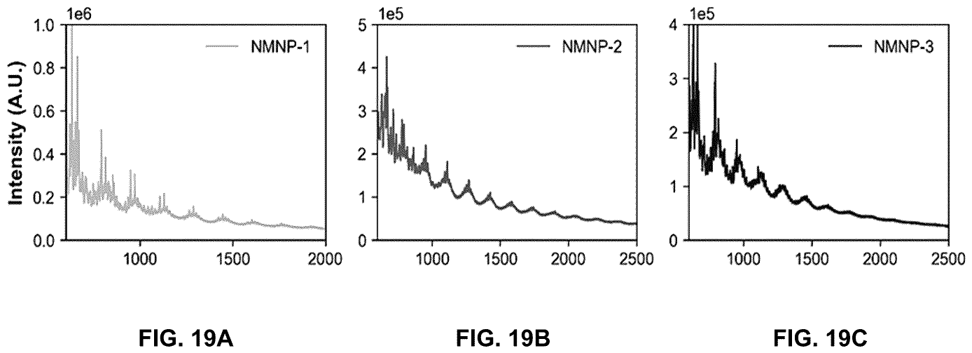

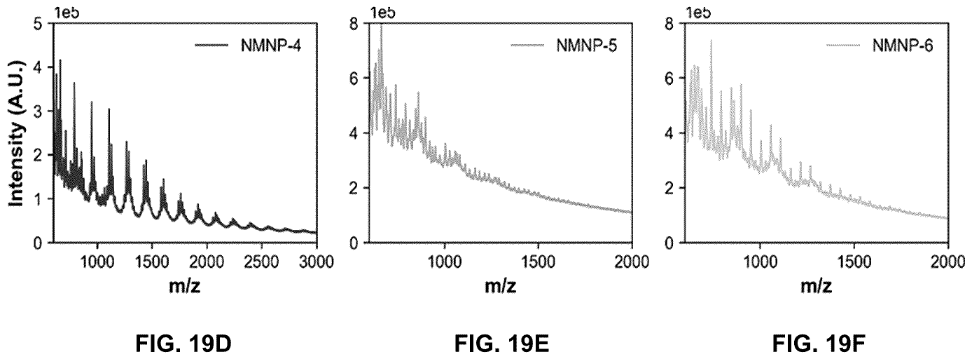

Typical synthetic allomelanin analogues are oxidatively polymerized from the 1,8-dihydroxynapthalene (1,8-DHN) monomer precursor, which self-assemble into spherical, dark pigmented nanoparticles, poly-1,8-DHN (p-1,8-DHN).¹⁰ To address the limitations of the dark pigmentation, alternate allomelanin-like nanoparticles were explored by oxidatively polymerizing commercially-available isomers of 1,8-DHN and screening for different pigmentation (see FIGS. 18A-18I, providing polymerization data for 1,3-DHN (FIG. 18A); 1,4-DHN (FIG. 18B); 1,5-DHN (FIG. 18C); 1,6-DHN (FIG. 18D); 1,7-DHN (FIG. 18E); 1,8-DHN (FIG. 18F); 2,3-DHN (FIG. 18G); 2,6-DHN (FIG. 18H); and 2,7-DHN (FIG. 18I) monomers). Our aim was to determine which isomers, upon polymerization, yielded a nanomaterial that could still enable visualization of a fluorometric or colorimetric response without any additional purification step. While oxidative polymerization of many of the isomers led to the typical dark pigmentation of the p-1,8-DHN, there were two isomers that exhibited light-colored pigmentation. These two isomers were 2,3-DHN (FIG. 18G) and 2,6-DHN (FIG. 18H). There were also isomers that, upon polymerization, led to more red pigmentation including 2,7-DHN (FIG. 18I), which also showed promise.

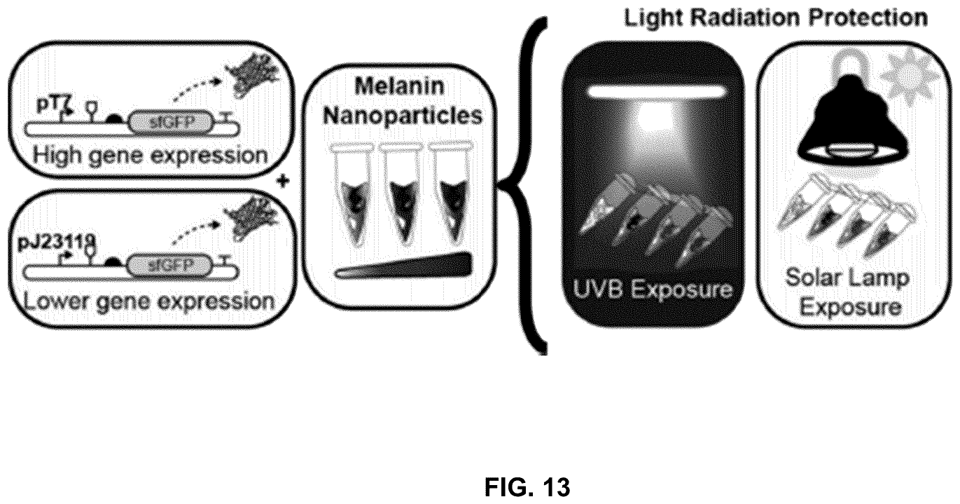

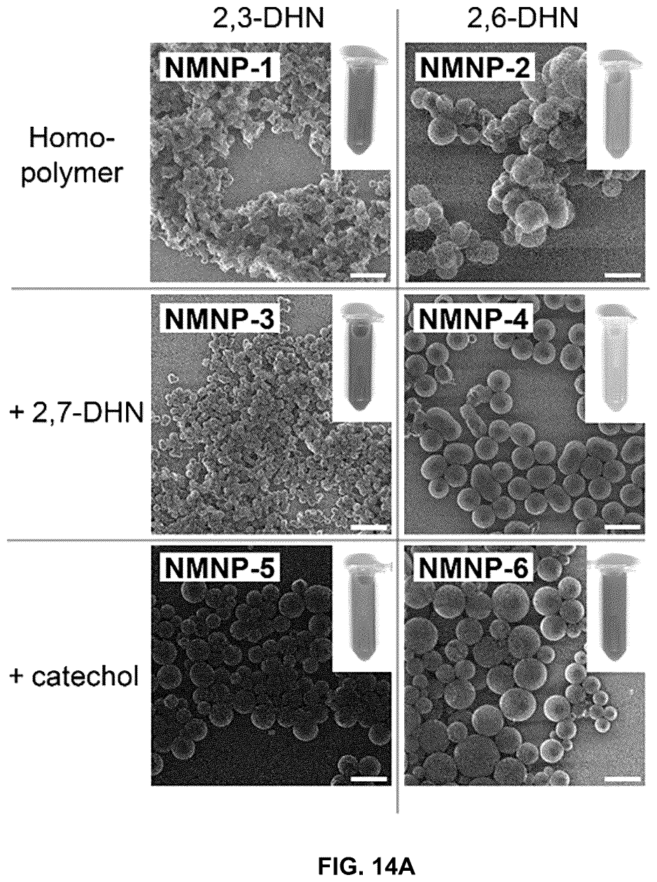

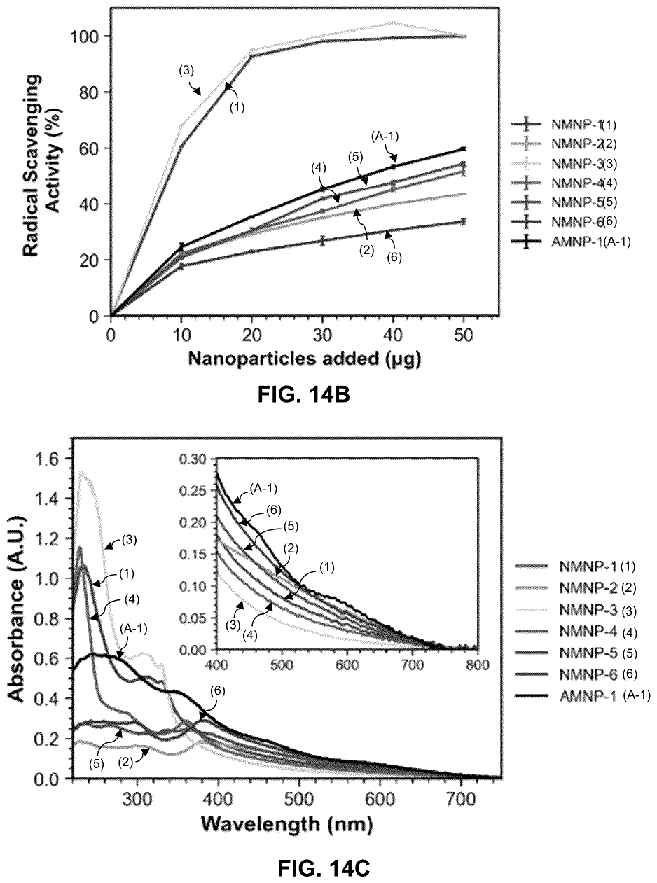

To gauge compatibility of these polymerized isomeric analogues of p-1,8-DHN, a small library of nanoparticles were generated by homopolymerizing and co-polymerizing 2,3-DHN and 2,6-DHN with other promising monomers. This library of melanin-like nanoparticles was called neo-melanin nanoparticles (NMNPs) and yielded nanoparticles with diverse optical pigmentations (FIG. 14A). NMNP-1 and NMNP-2 are homo-polymerizations of 2,3-DHN and 2,6-DHN, respectively. NMNP-3 and NMNP-4 are copolymerization of 2,7-DHN with 2,3-DHN and 2,6-DHN, respectively. Lastly, NMNP-5 and NMNP-6 are copolymerization of catechol with 2,3-DHN and 2,6-DHN, respectively. The monomers, 2,7-DHN and catechol, were strategically chosen to be co-polymerized into the 2,3-DHN and 2,6-DHN. The 2,7-DHN monomer was chosen because upon polymerization, it yielded a red pigment and it was hypothesized that it would increase the visible light absorbance of the NMNP which would enhance its UV-protective capabilities, while still making it amenable for the visualization of cell-free sensors. In a similar vein, catechol was also chosen because it would increase the light absorbance of the NMNP to improve its UV-protective capabilities, since catechol is known to polymerize into a dark, black pigment. However, by co-polymerization of small amounts of catechol, the resulting NMNPs still were lightly pigmented enough to be amenable to cell-free sensors.

To test these hypotheses, the UV-Vis absorption spectra for all NMNPs and p-1,8-DHN were characterized (FIG. 14C). In agreement with the proposed hypothesis for catechol co-polymerization, the UV-Vis absorbance spectra of FIG. 14C showed that NMNP-5 and NMNP-6 had a higher absorption in the visible region in comparison to NMNP-1 and NMNP-2, respectively. In contrast, the UV-Vis absorption spectra showed that adding 2,7-DHN did not enhance the visible light absorption for NMNP-3 and NMNP-4. Rather, there was an increased absorbance in the UV-region for NMNP-3 and NMNP-4, which could enhance the UV-light protection capabilities of these nanomaterials; however, this also indicated that there may have been high quantities of DHN monomer within the nanoparticle which can pose potential toxicity effects for the cell-free systems.

It is hypothesized that melanin's radiation protective properties originate not only from their broadband light absorption properties, but also from its high radical scavenging activity attributed to its high intrinsic radical content and its reversible redox properties that allows it to quench or stabilize free radicals. This antioxidant activity of the NMNP library generated in this example was characterized using a 2,2′-azinobis(3-ethylbenzothiazoline-6-sulfonic acid) (ABTS) radical scavenging assay (FIG. 14B). As depicted in FIG. 14B, NMNP-1 and NMNP-3 exhibited high radical scavenging activity, significantly surpassing the rest of the NMNPs tested. Notably, these two materials contained 2,3-DHN monomer. Copolymerization of the 2,3-DHN with the catechol (NMNP-5) however led to a significant decrease in radical scavenging activity in comparison to the 2,3-DHN homopolymer (NMNP-1). This was hypothesized to be attributed to the increased crosslinking within NMNP-5 facilitated by the catechol monomer that is believed to lead to not only the increase in the visible light absorption of the material but also a decrease in the interfacial interactions and quenching of the free radicals. A similar decrease in radical scavenging was also observed with co-polymerizing catechol with 2,6-DHN (NMNP-6) in comparison to the 2,6-DHN homopolymer (NMNP-2). There was also a slight increase in radical scavenging activity when 2,6-DHN and 2,7-DHN were co-polymerized (NMNP-5) in contrast to NMNP-2. With the exception of NMNP-1 and NMNP-3, the rest of the NMNPs tested showed radical scavenging activity that were comparable or below the activity of the synthetic allomelanin nanoparticles, p-1,8-DHN. Overall, the NMNPs still exhibited melanin's broadband absorption properties and antioxidant activity with the added benefit of tunable pigmentation making them promising candidates for the UV-protection of cell-free systems.

### Example 4.2 Candidate Neo-Melanin Nanoparticles Show Compatibility with Cell-Free Expression

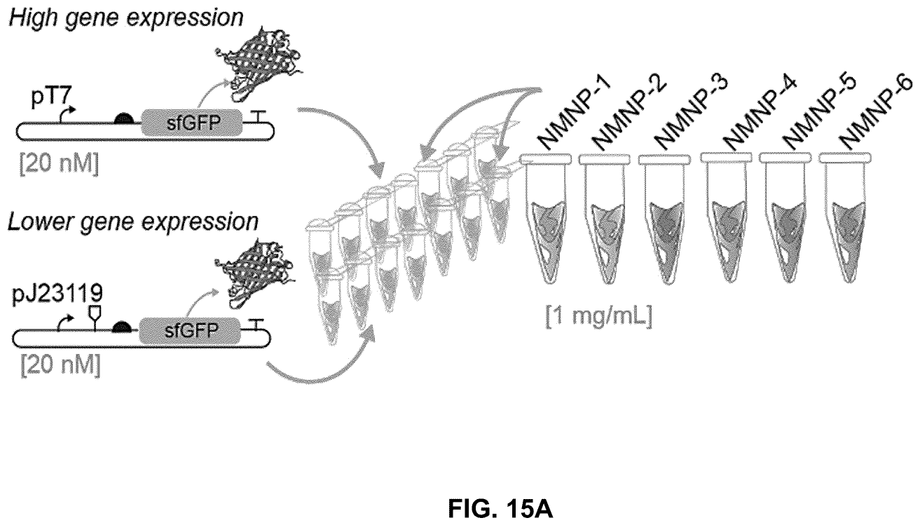

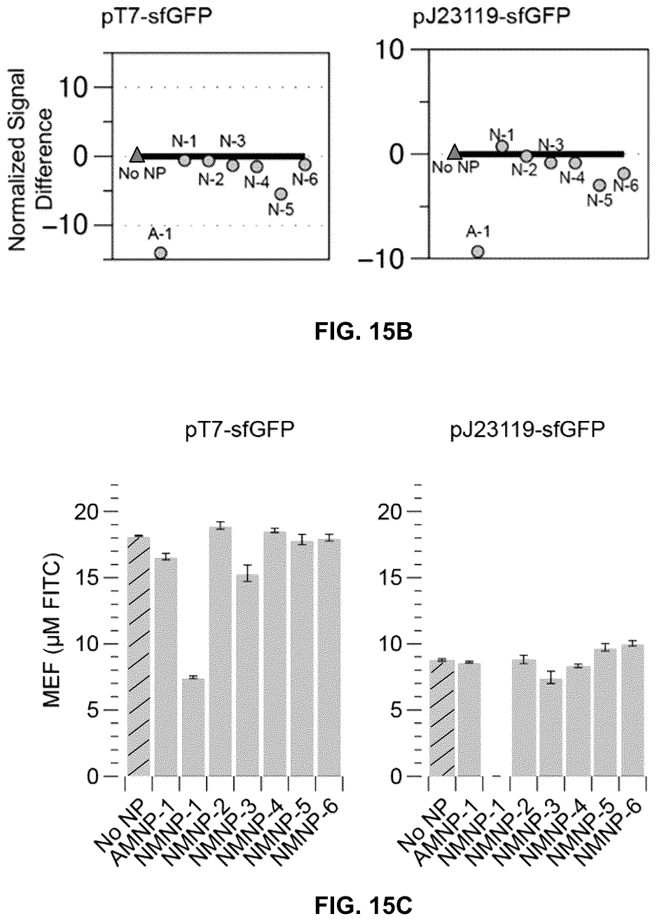

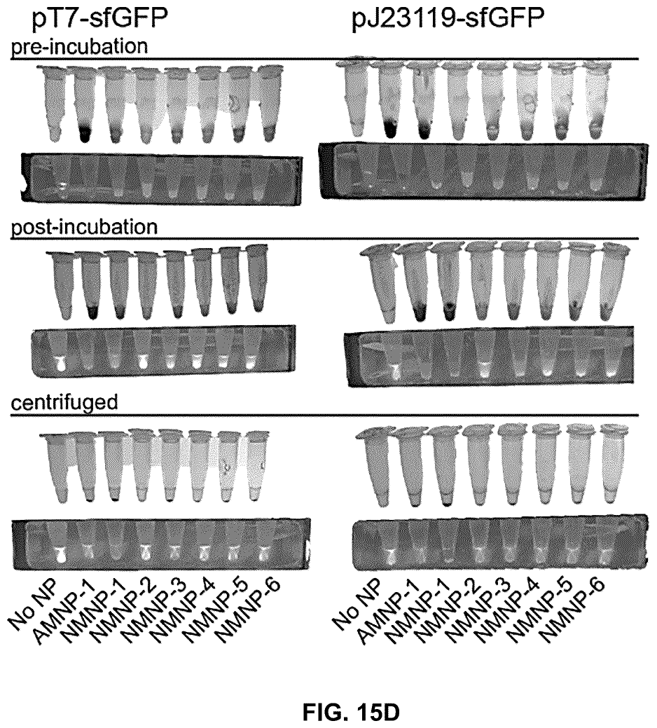

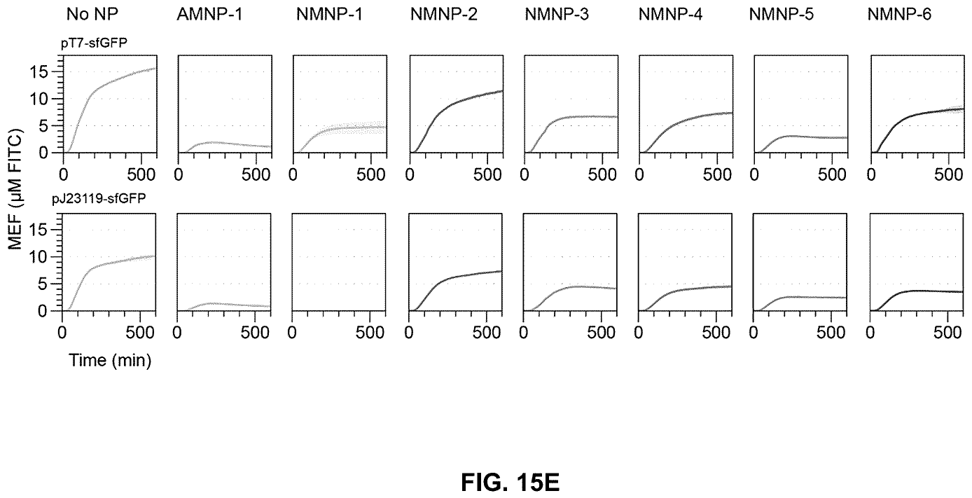

Next, compatibility of the NMNPs with cell-free expression (CFE) and their optical clarity in reactions was evaluated to select candidates for the UV-protection of cell-free sensors. As depicted in the schematic of FIG. 15A, NMNPs were added into E. coli lysate-based cell-free reactions at concentrations of 1 mg/mL where super folder GFP (sfGFP) production was evaluated post incubation for 20 h under a strong T7 RNAP promoter (pT7) and weaker E. coli J23119 RNAP promoter (pJ23119). To assess whether the neomelanin NPs were more adaptable for plate reader assays and visualizable in tubes than the darker colored allomelanin NP, AMNP-1, the signal difference between incubated reaction endpoint fluorescence values (NPs removed by centrifugation) and the endpoint fluorescence of the kinetic traces taken on a plate reader (NPs retained in reaction) was determined. These were then normalized to the no nanoparticle condition to create a threshold at zero (No NP) that describes the ability to read reactions containing melanin NPs on a plate reader (FIG. 15B). Particles closer to the zero line had a value nearer to the no NP control reactions, indicating a lesser signal difference in reactions that were analyzed after removal of nanoparticles and those analyzed with nanoparticles in solution. All NMNPs were more optically clear than AMNP-1, showing that the tested NMNPs were compatible with plate reader assays (FIG. 15B).

For the pT7 and pJ23119 reporter, NMNP-2, NMNP-3, NMNP-4, NMNP-5, and NMNP-6 all showed sfGFP expression comparable to that of the no nanoparticle (No NP) control (FIG. 15C). NMNP-1 inhibited both pT7-sfGFP and pJ23119-sfGFP expression. To further understand visualization, reactions containing each NMNP, AMNP-1, and no nanoparticles were imaged to assess if fluorescence could be observed after running the reactions (FIG. 15C). As shown in FIG. 15D, AMNP-1 blocked blue light from entering the tubes and made visualization difficult, while the tested NMNPs appeared to partially auto fluoresce pre-incubation. However, signal differences in post incubation without centrifugation were easier to visualize than in reactions containing AMNP-1. In the presence of the lighter-pigmented NMNPs, sfGFP expression could be analyzed using a plate reader, though with a reduction in apparent signal, unlike AMNP-1 previously reported (FIG. 15E). From this compatibility screen, NMNP-2 and NMNP-6 were selected as lighter and darker NP candidates, respectively, for further investigation.

### Example 4.3 Candidate Neo-Melanin Nanoparticle Protect Cell-Free Gene Expression from Light Exposure Events

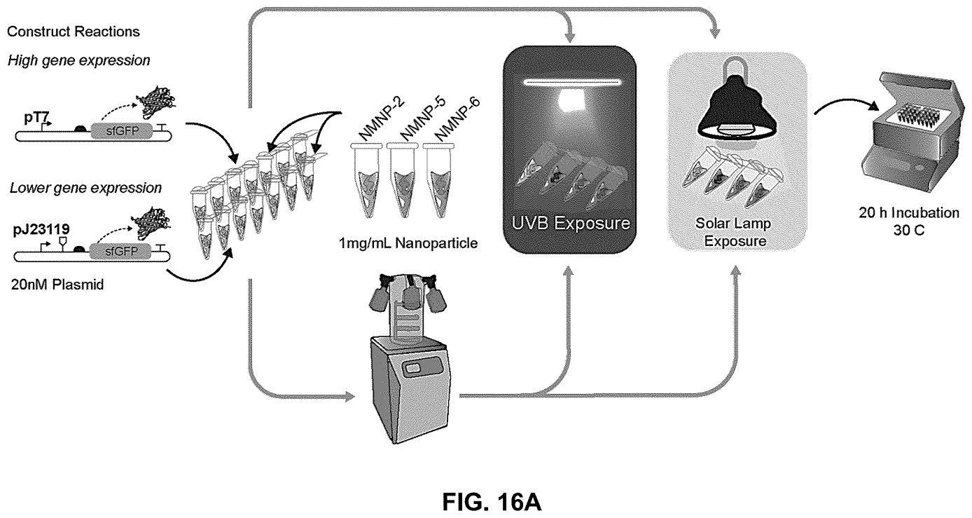

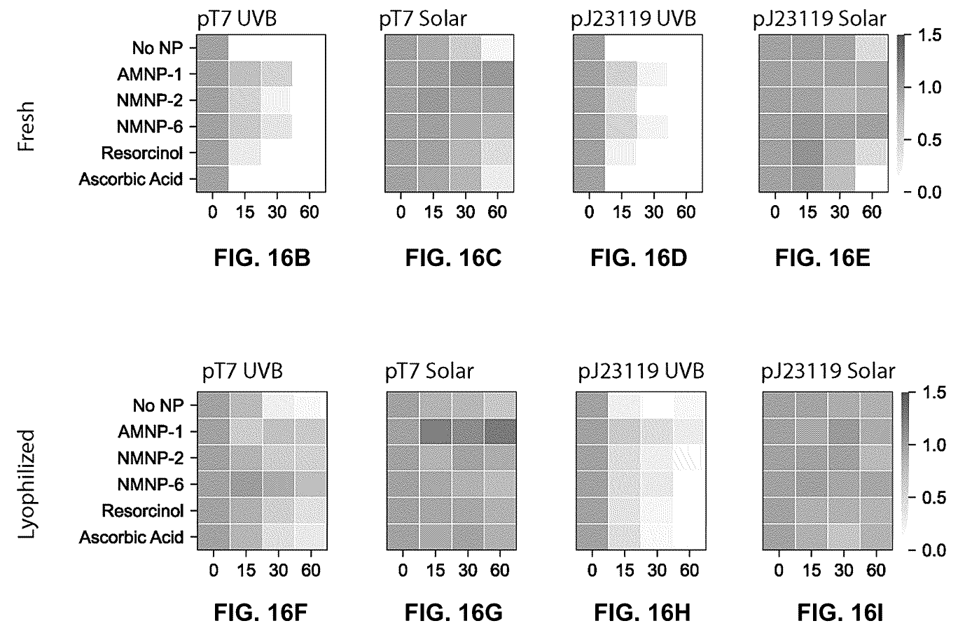

Next, NMNP-2 and NMNP-6 were evaluated for CFE protection from light exposure events. As exemplified in the schematic of FIG. 16, 20 μL cell-free reactions were constructed using pT7-sfGFP or pJ23119-sfGFP expression plasmids with the AMNP/NMNP additives at 1 mg/mL, antioxidants resorcinol and ascorbic acid at 1 mg/mL, and no additive control. These reactions were then exposed to UVB or solar lamp sources for 0, 15, 30, and 60 minutes on a cooling plate set to 4° C. as described in Examples 1-3, above. These reactions were incubated at 30° C. for 20 h post light exposure and characterized for sfGFP fluorescence (FIG. 16A).

The results show that under both UV and solar lamp exposure, the addition of NMNPs provided increased radiation protection, like AMNP-1, over an extended period in both pT7 and pJ23119 sfGFP expression as compared to the no NP condition (FIGS. 16B-16E). Additionally, in these experiments, resorcinol was shown to provide some protection, though weaker than the NP additives, while ascorbic acid offered minimal to no protection. For pT7-sfGFP UVB exposure, AMNP-1 retained about 57% of the initial signal, while NMNP-2 and NMNP-6 retained about 24% and 37% of the initial signal respectively, compared to the no NP control which retained only about 2% of its initial signal (FIG. 16B). pT7-sfGFP solar-exposed reactions containing AMNP-1, NMNP-2, and NMNP-6 also showed retained function, with initial signal retention rangom from about 87-105% at 60 min (FIG. 16C). For pJ23119 UVB exposure, AMNP-1, NMMP-2, and NMNP-6 retained about 22%, 9.7%, and 22% of their initial signals at 30 minutes of exposure, respectively (FIG. 16D). Here, resorcinol, ascorbic acid, and the no NP control retained about 7%, 0.9% and 0.9% respectively (FIG. 16D). Solar lamp-exposed pJ23119 reactions containing AMNP-1, NMNP-2, and NMNP-6 retained about 94%, 89%, and 100% initial signal at 60 minutes, respectively, while no NP and resorcinol conditions retained about 46% initial signal, with ascorbic acid showing the lowest percent initial signal at about 12% (FIG. 16E). These results suggest that neomelanin additives offered protection comparable to darker melanins and better protection than other antioxidants or no additives.

Reactions were also lyophilized and exposed to light conditions, which were previously determined to add to the protective properties of the melanin additives (FIGS. 16F-16I). Here, it was observed that AMNP-1, NMNP-2, and NMNP-6 in conjunction with lyophilization aided in protection against UVB exposure, while solar exposure sfGFP expression levels were similar between no NP and NP additive conditions. For pT7-sfGFP CFE UVB exposure, AMNP-1, NMNP-2 and NMNP-6 retained between about 59-78% initial signal after lyophilization at 60 min exposure times, with antioxidants retaining about 33-43% initial reaction compared to only about 18% retained in the no NP control (FIG. 16F). For lyophilized pT7-sfGFP reaction solar exposure, all melanin NP conditions and antioxidants retained higher signal than the no NP control at 60 min (about 73%) with a range of retained initial signals from about 80-130% (FIG. 16G). PJ23119-sfGFP reactions lyophilized and exposed to UVB irradiation were more protected by ANMNP-1, NMNP-2, and NMNP-6 than antioxidants and no NP conditions with initial signal retention ranging from about 28-46% (FIG. 16H). For lyophilized, solar light exposed, pJ23119-sfGFP reactions, all reactions retained high percentages of initial signal ranging from about 82-100% through the duration of exposure. These results indicate that NMNPs are compatible with lyophilization and offer improved light protection for freeze-dried cell-free reactions.

### Example 4.4 Candidate Neo-Melanin Nanoparticles Protect Cell-Free Sensors from Light Exposure Events

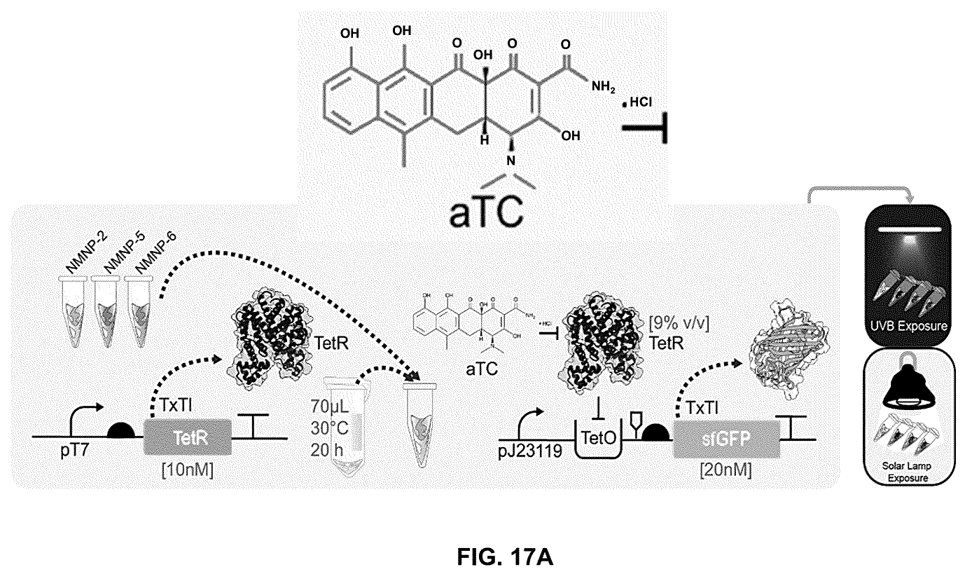

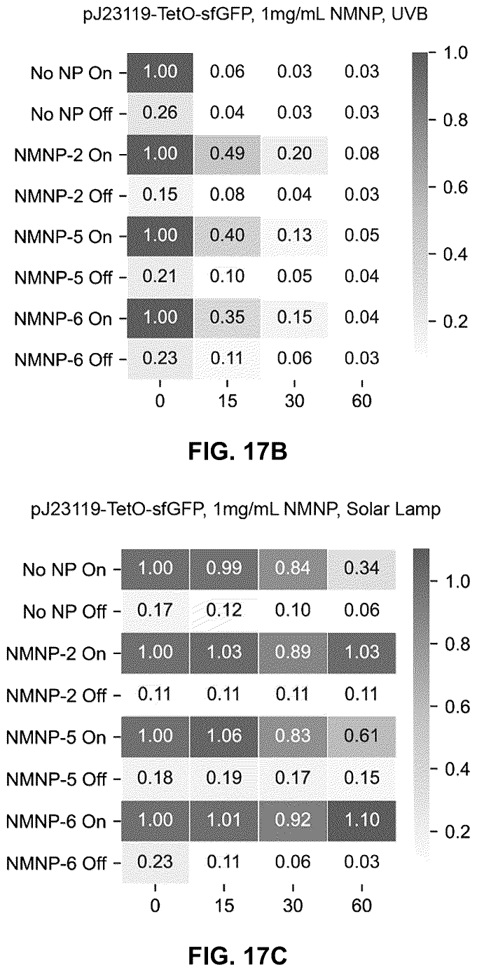

Next, the candidate NMNPs were utilized as a protective additive for cell-free sensors. It was hypothesized that the engineered pigmentation of NMNPs would enable real-time visualization of cell-free sensors. The sensor that was used in the study is TetR, which responds to anhydrotetracycline (aTC) (FIG. 17A). The mechanism here is that TetR, synthesized by overnight CFPS reactions, is added to a cell-free reaction containing reporter DNA with the TetO operator (DNA Sequence that binds TetR) controlling sfGFP expression. When the TetR is present in the absence of ligand (sensor off conditions) gene expression is repressed due to blocking of RNAP. When aTC is present, the TetR unbinds the TetO operator and allows for gene expression to occur which will make sfGFP. The entire reaction, [TetR+Reporter+aTC] and [TetR+Reporter-aTC], were constructed before exposure and then were allowed to run 20 h before being exposed to irradiation. This method of exposure was selected over irradiating just the [TetR+Reporter] to irradiation then inducing the sensor with the atC ligand because this selected method would make more sense for lyophilized reactions. The sfGFP expression were normalized to their expression in the no light exposure, “on” condition for each respective nanoparticle condition.

The results showed that the pJ23119-TetO-sfGFP sensor was more sensitive to UVB lamp exposure than solar lamp exposure, with the sensor losing near complete expression after 15 minutes of UVB exposure. The NMNP-2, NMNP-5, and NMNP-6 all exhibited similar protective effects against UVB, retaining about 49%, 40%, and 35% of sfGFP expression, respectively, in the sensor “on” condition after 15 minutes of UVB irradiation (FIG. 17B). In the solar lamp exposure condition, the no NP control showed progressive loss in sensor activity as exposure time was increased, with the sensor only exhibiting about 34% or sfGFP expression during the “on” sensor condition. Addition of NMNP additives demonstrated increased sensor expression at high solar lamp exposure times (FIG. 17C). NMNP-2 and NMNP-6 showed near complete preservation of sfGFP expression after 60 minutes of exposure time compared to their no exposure control. NMNP-5 showed slightly lesser protective effects as compared to NMNP-2 and NMNP-6, preserving about 61% of the original sfGFP expression, which was still significantly higher than the no NP control. These results supported the trend seen previously in this Example 4, where NMNP-2 and NMNP-6 showed the best radiation protective capabilities of the samples tested in this Example, followed by NMNP-5.

### Discussion Corresponding to Example 4

In some embodiments, it appears that the NMNPs that contained 2,3-DHN had lower compatibility with cell-free expression, which was reflected in the relatively lower performance of NMNP-1, NMNP-3, and NMNP-5 in comparison to the NMNP-2, NMNP-4, and NMNP-6, which contained 2,6-DHN. The results also showed that the co-polymerization of catechol with 2,3-DHN, however, improved the cell-free compatibility. On the other hand, co-polymerization of 2,7-DHN with 2,6-DHN (NMNP-6) decreased the sfGFP expression in comparison to the 2,6-DHN homopolymer (NMNP-2). Similarly to the pT7 promoter, the pJ23119-sfGFP in the presence of NMNPs could be observed optically under UV light without the need for spinning the particles down.

### Exemplary Experimental Aspects for Example 4

Materials: 1,8-dihydroxynapthalene (1,8-DHN) (95+%, Catalog No. 76152) was purchased from Matrix Scientific. 2,3 dihydroxynapthalene (2,3DHN) (98%, Catalog No. A11307) was purchased from Alfa Aesar. Dopamine hydrochloride (99%, Catalog No. A11136.22), 2,2-azino-bis-3-ethylbenzothiazoline-6-sulphonic acid (ABTS), sodium periodate (NaIO₄) (98.5%), sodium hydroxide (NaOH), potassium persulfate, HPLC grade acetonitrile (ACN) and HPLC grade ethanol were purchased from ThermoFisher Scientific. L-ascorbic acid were purchased from Sigma Aldrich. Tetraethyl orthosilicate (TEOS) was purchased from Acros Organics. Grids for transmission electron microscopy (TEM) were purchased from Electron Microscopy Sciences (EMS).

Instrumentation: Scanning transmission electron microscopy (STEM) micrographs were taken with an SE-detector on Hitachi HD2300 at an accelerating voltage of 200 kV. Hydrodynamic radii were measured with a Wyatt Technology DynaPro NanoStar dynamic light scattering (DLS). Zeta potential of the nanoparticles were measured using Malvern Instruments Ltd, Nano Zetasizer in ultrapure water at room temperature. UV-Vis spectra of the NMNPs were measured at 0.01 mg/mL in ultrapure water on a ThermoScientific Nanodrop 2000c spectrophotometer. ABTS colorimetric radical scavenging assay measurements were acquired using PerkinElmer Enspire Multimode Plate Reader.

### Synthesis of Melanin Nanoparticles:

The NMNPs were synthesized in a similar fashion to AMNP-1 in Zhou et al.²² NMNP-1 was synthesized by dissolving 150 mg of 2,3-DHN in 7.5 mL of ACN before adding 142.5 mL of ultrapure water. Then, the reaction was injected with a 1 mL solution of NaIO₄ (0.47 mmol, 100.15 mg) in ultrapure water and left to stir on a stir plate for 20 hours. NMNP-2 was synthesized by dissolving 150 mg of 2,6-DHN in 7.5 mL of ACN before adding 142.5 mL of ultrapure water. Then, the reaction was injected with a 2 mL solution of NaIO₄ (0.94 mmol, 200.3 mg) in ultrapure water and left to stir on a stir plate for 20 hours. A higher concentration of oxidant (NaIO₄ at 0.94 mmol, 200.3 mg)) was used for NMNP-2 as compared to the other nanoparticles because it improved the yield of NMNP-2. While polymerizing with a 1 mL solution of NaIO₄ (0.47 mmol, 100.15 mg) is sufficient to generate NMNP-2, a higher concentration of oxidant was used in this Example 4 for efficiency. NMNP-3 was synthesized by dissolving 75 mg of 2,3-DHN and 75 mg of 2,7-DHN in 7.5 mL of ACN before adding 142.5 mL of ultrapure water. Then, this mixture was injected with a 1 mL solution of NaIO₄ (0.47 mmol, 100.15 mg) in ultrapure water and left to stir on a stir plate for 20 hours. NMNP-4 was synthesized by dissolving 75 mg of 2,6-DHN and 75 mg of 2,7-DHN in 7.5 mL of ACN before adding 142.5 mL of ultrapure water. Then, the reaction was injected with a 1 mL solution of NaIO₄ (0.47 mmol, 100.15 mg) in ultrapure water and left to stir on a stir plate for 20 hours. NMNP-5 was synthesized by dissolving 125 mg of 2,3-DHN and 25 mg of catechol in 7.5 mL of ACN before adding 142.5 mL of ultrapure water. Then, the reaction was injected with a 1 mL solution of NaIO₄ (0.47 mmol, 100.15 mg) in ultrapure water and left to stir on a stir plate for 20 hours. NMNP-6 was synthesized by dissolving 125 mg of 2,6-DHN and 25 mg of catechol in 7.5 mL of ACN before adding 142.5 mL of ultrapure water. Then, the reaction was injected with a 1 mL solution of NaIO₄ (0.47 mmol, 100.15 mg) in ultrapure water and left to stir on a stir plate for 20 hours. All NMNP particles isolated and washed three times via centrifugation with ultrapure water (11000 rpm, 10 minutes).

Synthesis of p-1,8-DHN. p-1,8-DHN was synthesized following the procedure outlined in Zhou et al.²² p-1,8-DHN was synthesized by dissolving 150 mg of 1,8-DHN in 7.5 mL of ACN before adding 142.5 mL of ultrapure water. Then, the reaction was injected with a 1 mL solution of NaIO₄ (0.47 mmol, 100.15 mg) in ultrapure water and left to stir on a stir plate for 20 hours. The particles were isolated and washed three times via centrifugation with ultrapure water (11000 rpm, 10 minutes).

ABTS Radical Scavenging Assay: An 2,2′-Azino-bis(3-ethylbenzothiazoline-6-sulfonic acid) diammonium salt (ABTS) radical scavenging activity of the NMNPs was based off a previously reported procedure with modifications.⁴⁹ Briefly, a bulk solution containing potassium persulfate (2.45 mM) and ABTS (7 mM) was prepared and left in the dark for 16 hours. This ABTS-potassium persulfate bulk solution was then diluted with ultrapure water such that the final stock solution contained 0.2 mM ABTS. Two duplicate sets of melanin nanoparticle solutions constituting of 10, 20, 30, 40, and 50 μg of nanoparticles in 100 μL of ultrapure water were prepared. In one set, 1.5 mL of the diluted ABTS stock solution (0.2 mM) was added and left in the dark for 20 minutes. After 20 minutes, all solutions were read on a plate reader at 734 nm (A₁) To the other set of melanin nanoparticle solutions, 1.5 ml of water was added to each make “blank” solutions and its absorbance at 734 nm was measured (A₂). Finally, 1.5 mL of the diluted ABTS stock solution (0.2 mM) was added to 100 μL of water and its absorbance at 734 nm was measured (A₀) To calculate the radical scavenging activity at each concentration of nanoparticles added, the following equation was utilized:

$$A B T S ( \% ) = 1 - ( \frac{( {A}_{1} - {A}_{2} )}{{A}_{0}} ) \times 100 \%$$

### Cell Free Expression Reactions:

E. coli lysate was prepared using strain BL21 (DE3)* with previously published protocols. See, Silverman et al., (ACS Synth. Biol. 2019, 8 (2), 403-414). Cell-free reactions were prepared using a PANOx reagent system, with reaction construction excel templates and solution preparation methods given in Silverman et al.^{37,44} This systems uses a salt solution containing 8 mM magnesium glutamate, 10 mM ammonium glutamate, 130 mM potassium glutamate; transcription master mix with 1.2 mM ATP, 0.850 mM GTP, 0.850 mM UTP, 0.850 mM CTP, 72 μM folinic acid, 0.171 mg/mL tRNA; amino acids solution with 2 mM of each amino acid; energy solution of 30 mM PEP; and cofactor solution with 0.33 mM NAD, 0.27 mM CoA, 4 mM oxalic acid, 1 mM putrescine, 1.5 mM spermidine, 57 mM HEPES. This mixture accounts for 30% of the reaction volume. Cell extract was also used at 30% of the reaction volume. For each experiment, DNA and the melanin nanoparticles were diluted in the water component of the reaction to make up the additional 40% reaction volume. Except for FIGS. 17A-17C, 20 nM of plasmids pJ23119-sfGFP and pT7-sfGFP DNA was used in all experiments.

Overnight cell free gene expression reactions with and without melanin nanoparticles were prepared by assembling all components on ice at a total volume of 20 μL and incubated at 30° C. for 20 h post light exposure described in UVB and Solar Lamp Exposure, below. The reactions were then spun on a centrifuge for 20 minutes at 3700 rpm to remove the particles from the reaction. 15 μL of supernatant was then collected in separate PCR tubes to ensure minimal residual nanoparticles were plated. 5 μL of each replicate was then plated in a 384 low volume well-plate (Corning® CLS3842) and fluorescence was measured plate reader set to excitation/emission 485 nm/520 nm and read from the bottom at gain 50 or 60 and ambient temperature. For plate reader assays, CFE reactions were constructed at 20 UL and 5 μL of each reaction was plated and read for 10 h at 30° C. using excitation/emission 485 nm/520 nm.

Lyophilized reactions were prepared on ice as above, snap cooled in liquid N₂ and then lyophilized using a Labconco FreeZone 2.5 Liter-84C Benchtop Freeze Dryer for 16 hours. Reactions were then immediately exposed to light conditions in UVB and Solar Lamp Exposure. Reactions were then rehydrated with 20 μL miliQ water, spun with a centrifuge, mixed with a pipette and then incubated for an additional 20 h at 30° C.

Data was calibrated to Mean Equivalent Fluorescence (MEF) using FITC calibration and this FITC calibrated data was used to calculate the normalized expression. Normalized expression is calculated as the endpoint GFP expression of the light exposure condition divided by the non-light exposed (t=0 min) condition for no nanoparticle, AMNP-1, AMNP-2, and PDA containing samples. This provides fractional reduction in expression due to light exposure. All experiments were done in triplicates. Data was then analyzed in python (see Data Processing, below).

UVB and Solar Lamp Exposure: In each of the experiments with UVB or solar lamp exposure, reactions were placed on a cooling plate set to 4° C. to reduce effects of heating the reaction with light and prevent gene expression before incubation, which could lead to unintended photobleaching of the reporter protein. UV/AB exposure of all sources were measured with a Sper Scientific UV/AB Light Meter (850009). UVB lamp (Spectronics X-15B, 15W, 312 nm) was used and reactions were placed directly on the cooling plate with the lamp sitting approximately 2.5 cm above providing an average UV/AB exposure between approximately 6000-7000 μW/cm², with some variability in measurement. A box was used to cover the exposure set up. For solar lamp exposure a solar lamp (MIXJOY Reptile Heat Lamp, 160 W) was used with the lamp placed 30 cm above the reactions corresponding to approximately 2500-3500 μW/cm² UV/AB exposure and temperature of 31° C. (88° F.) due to radiative heat from the lamp. This solar simulating lamp features a brightness of 1200 lumens and covers the full UVA, UVB, visible and infrared spectrum. This solar lamp condition was chosen to best represent solar exposure on a hot, sunny day with a high UVB exposure of 265 μW/cm² according to the manufacturer's description. For reference, in particularly hot climates, UVB exposure can be up to 350-450 μW/cm^{2. 50-52} Notably, in nature, UVB exposure can vary greatly by location, weather, and time of day. Reactions for each time point were made separately so that each condition represents continuous exposure. The reactions were then placed on the cooling plate starting with the 60 min time point reactions, then after 30 minutes of time passing, the 30 min exposure condition were added, after an additional 15 minutes of time passing the reactions to be exposed for 15 minutes were added and sat under the light condition for the remainder of the time. Reactions were then processed in accordance with the methods described in Cell Free Gene Expression Reactions.

Data Processing: Python files used to generate figures from the data files and analyze DLS data are provided at the github in Data Availability. All heat maps are presented as normalized expression to the no light exposure controls for each figure to make it easier to distinguish patterns in the data, FITC normalized plate reader values are presented similarly in Supplemental Information files and raw plate reader values are presented in excel files within Supplementary Data.

Plasmids Used in Example 4: Plasmid pJL1 (Addgene #69496) was used for pT7-sfGFP expression, and pJBL7010 (Addgene #136942) was used for pJ23119-sfGFP expression. The TetR biosensor was purchased from IDT as gBlocks, a TetR transcription factor producting template and a Tet operator containing sfGFP reporter template. A table of plasmids and g blocks used in this study can be found in Supplemental Information.

### REFERENCES CORRESPONDING TO EXAMPLE 4

(1) Guo, L., et al., Recent Advances and Progress on Melanin: From Source to Application. Int J Mol Sci 2023, 24 (5), 4360.
(2) Tran-Ly, A. N., et al., Microbial Production of Melanin and Its Various Applications. World J Microbiol Biotechnol 2020, 36 (11), 170.
(3) Mattoon, E. R.; Cordero, R. J. B.; Casadevall, A. Fungal Melanins and Applications in Healthcare, Bioremediation and Industry. J Fungi (Basel) 2021, 7 (6), 488.
(4) Cao, W., et al., Unraveling the Structure and Function of Melanin through Synthesis. J. Am. Chem. Soc. 2021, 143 (7), 2622-2637.
(5) Fordjour, E., et al., Metabolic Engineering of Escherichia Coli BL21 (DE3) for de Novo Production of I-DOPA from d-Glucose. Microb Cell Fact 2019, 18 (1), 74.
(6) Surwase, S. N.; Jadhav, J. P. Bioconversion of L-Tyrosine to I-DOPA by a Novel Bacterium Bacillus Sp. JPJ. Amino Acids 2011, 41 (2), 495-506.
(7) Wei, T., et al., Genome Engineering Escherichia Coli for L-DOPA Overproduction from Glucose. Sci Rep 2016, 6 (1), 30080.
(8) Yoshida, H., et al., Production of 3,4-Dihydroxyphenyl-I-Alanine (I-DOPA) by Pseudomonas melanogenum. Agricultural and Biological Chemistry 1974, 38 (2), 455-462.
(9) Walker, K. T., et al., Self-Pigmenting Textiles Grown from Cellulose-Producing Bacteria with Engineered Tyrosinase Expression. Nat Biotechnol 2024, 1-10.
(10) El-Bialy, H. A., et al., Microbial Melanin Physiology under Stress Conditions and Gamma Radiation Protection Studies. Radiation Physics and Chemistry 2019, 162, 178-186.
(11) Corani, A., et al., Superior Photoprotective Motifs and Mechanisms in Eumelanins Uncovered. J. Am. Chem. Soc. 2014, 136 (33), 11626-11635.
(12) Dadachova, E., et al., Ionizing Radiation Changes the Electronic Properties of Melanin and Enhances the Growth of Melanized Fungi. PLOS ONE 2007, 2 (5), e457.
(13) Dadachova, E.; Casadevall, A. Melanin and Resistance to lonizing Radiation in Fungi. In Extremophiles Handbook; Horikoshi, K., Ed.; Springer Japan: Tokyo, 2011; pp 1147-1157.
(14) Galván, I.; Solano, F. Bird Integumentary Melanins: Biosynthesis, Forms, Function and Evolution. IJMS 2016, 17 (4), 520.
(15) Koike, S.; Yamasaki, K. Melanogenesis Connection with Innate Immunity and Toll-Like Receptors. IJMS 2020, 21 (24), 9769.
(16) Lichtenegger, H. C., et al., High Abrasion Resistance with Sparse Mineralization: Copper Biomineral in Worm Jaws. Science 2002, 298 (5592), 389-392.
(17) McCallum, N. C., et al., Allomelanin: A Biopolymer of Intrinsic Microporosity. J. Am. Chem. Soc. 2021, 143 (10), 4005-4016.
(18) Biyashev, D., et al., Topical Application of Synthetic Melanin Promotes Tissue Repair. npj Regen Med 2023, 8 (1), 61.
(19) Huang, Y., et al., Mimicking Melanosomes: Polydopamine Nanoparticles as Artificial Microparasols. ACS Cent. Sci. 2017, 3 (6), 564-569.
(20) Hu, J., et al., Polydopamine Free Radical Scavengers. Biomater. Sci. 2020, 8 (18), 4940-4950.
(21) Menichetti, A., et al., Melanin as a Photothermal Agent in Antimicrobial Systems. Int. J. Mol. Sci. 2024, 25 (16), No. 8975.
(22) Zhou, X., et al., Artificial Allomelanin Nanoparticles. ACS Nano 2019, 13 (10), 10980-10990.
(23) Warfel, K. F., et al., A Low-Cost, Thermostable, Cell-Free Protein Synthesis Platform for On-Demand Production of Conjugate Vaccines. ACS Synth. Biol. 2023, 12 (1), 95-107.
(24) DeWinter, M. A., et al., Point-of-Care Peptide Hormone Production Enabled by Cell-Free Protein Synthesis. ACS Synth. Biol. 2023, 12 (4), 1216-1226.
(25) Thavarajah, W., et al., Point-of-Use Detection of Environmental Fluoride via a Cell-Free Riboswitch-Based Biosensor. ACS Synth. Biol. 2020, 9 (1), 10-18.
(26) Gregorio, N. E., et al., Unlocking Applications of Cell-Free Biotechnology through Enhanced Shelf Life and Productivity of E. Coli Extracts. ACS Synth. Biol. 2020, 9 (4), 766-778.
(27) Thavarajah, W., et al., The Accuracy and Usability of Point-of-Use Fluoride Biosensors in Rural Kenya. npj Clean Water 2023, 6 (1), 1-8.
(28) Raeiszadeh, M.; Adeli, B. A Critical Review on Ultraviolet Disinfection Systems against COVID-19 Outbreak: Applicability, Validation, and Safety Considerations. ACS Photonics 2020, 7 (11), 2941-2951.
(29) Pereira, R. V., et al., Evaluation of the Effects of Ultraviolet Light on Bacterial Contaminants Inoculated into Whole Milk and Colostrum, and on Colostrum Immunoglobulin G. Journal of Dairy Science 2014, 97 (5), 2866-2875.
(30) Yemmireddy, V., et al., Effect of Ultraviolet Light Treatment on Microbiological Safety and Quality of Fresh Produce: An Overview. Front. Nutr. 2022, 9, 871243.
(31) Lee, J. W., et al., Deciphering UV-induced DNA Damage Responses to Prevent and Treat Skin Cancer. Photochem & Photobiology 2020, 96 (3), 478-499.
(32) IARC Working Group on the Evaluation of Carcinogenic Risks to Human, Radiation; International Agency for Research on Cancer: Lyon, France, 2012.
(33) De Jager, T. L. et al., Ultraviolet Light Induced Generation of Reactive Oxygen Species. In Ultraviolet Light in Human Health, Diseases and Environment; Ahmad, S. I., Ed.; Advances in Experimental Medicine and Biology; Springer International Publishing: Cham, 2017; Vol. 996, pp 15-23.
(34) Health Effects of Exposure to Low Levels of Ionizing Radiation: BEIR V; National Academies Press: Washington, D.C., 1990; p 1224.
(35) Nambiar, S.; Yeow, J. T. W. Polymer-Composite Materials for Radiation Protection. ACS Appl. Mater. Interfaces 2012, 4 (11), 5717-5726.
(36) Silverman, A. D., et al., Deconstructing Cell-Free Extract Preparation for in Vitro Activation of Transcriptional Genetic Circuitry. ACS Synth. Biol. 2019, 8 (2), 403-414.
(37) Oppezzo, O. J. Contribution of UVB Radiation to Bacterial Inactivation by Natural Sunlight. Journal of Photochemistry and Photobiology B: Biology 2012, 115, 58-62.
(38) Santos, A. L., et al., Contribution of Reactive Oxygen Species to UV-B-Induced Damage in Bacteria. Journal of Photochemistry and Photobiology B: Biology 2012, 117, 40-46.
(39) Cacciari, R. D., et al., Effect of UVB Solar Irradiation on Laccase Enzyme: Evaluation of the Photooxidation Process and Its Impact over the Enzymatic Activity for Pollutants Bioremediation. Amino Acids 2020, 52 (6), 925-939.
(40) McMillan, T. J., et al., Cellular Effects of Long Wavelength UV Light (UVA) in Mammalian Cells. Journal of Pharmacy and Pharmacology 2008, 60 (8), 969-976.
(41) Kulms, D., et al., Nuclear and Cell Membrane Effects Contribute Independently to the Induction of Apoptosis in Human Cells Exposed to UVB Radiation. Proceedings of the National Academy of Sciences 1999, 96 (14), 7974-7979.
(42) Garmyn, M.; Yarosh, D. B. The Molecular and Genetic Effects of Ultraviolet Radiation Exposure on Skin Cells. In Photodermatology; CRC Press, 2007.
(43) Kim, D. M.; Swartz, J. R. Regeneration of Adenosine Triphosphate from Glycolytic Intermediates for Cell-Free Protein Synthesis. Biotechnol. Bioeng. 2001, 74 (4), 309-316.
(44) Hunt, A. C., et al., Cell-Free Gene Expression: Methods and Applications. Chem. Rev. 2025, 125 (1), 91-149.
(45) Silverman, A. D., et al., Cell-Free Gene Expression: An Expanded Repertoire of Applications. Nat. Rev. Genet. 2020, 21 (3), 151-170.
(46) Kocalar, S., et al., Validation of Cell-Free Protein Synthesis Aboard the International Space Station. ACS Synth. Biol. 2024, 13 (3), 942-950.
(47) Noman, A., et al., Characterization of Physicochemical Properties of Melanin Produced by Gluconobacter Oxydans FBFS 97. Fermentation 2022, 8 (11), 574.
(48) MacCargar, B. Iguanas and Artificial Ultraviolet Light: How and How Much Made Simple—Well, Not Exactly Simple. Iguana 2003, 10 (3), 82-85.
(49) Mader, D. R.; Divers, S. J., Eds. Current Therapy in Reptile Medicine and Surgery; Saunders Elsevier; St. Louis, MO, 2014.
(50) Diehl, J. J. E., et al., A. B. A. Comparison of UV b Compact Lamps in Enabling Cutaneous Vitamin D Synthesis in Growing Bearded Dragons. Anim. Physiol. Nutr. 2018, 102 (1), 308-316.

### STATEMENTS REGARDING INCORPORATION BY REFERENCE AND VARIATIONS

All references throughout this application, for example patent documents including issued or granted patents or equivalents; patent application publications; and non-patent literature documents or other source material; are hereby incorporated by reference herein in their entireties, as though individually incorporated by reference, to the extent each reference is at least partially not inconsistent with the disclosure in this application (for example, a reference that is partially inconsistent is incorporated by reference except for the partially inconsistent portion of the reference).

The terms and expressions which have been employed herein are used as terms of description and not of limitation, and there is no intention in the use of such terms and expressions of excluding any equivalents of the features shown and described or portions thereof, but it is recognized that various modifications are possible within the scope of the invention. Thus, it should be understood that although the present invention has been specifically disclosed by preferred embodiments, exemplary embodiments and optional features, modification and variation of the concepts herein disclosed may be resorted to by those skilled in the art, and that such modifications and variations are considered to be within the scope of this invention. The specific embodiments provided herein are examples of useful embodiments of the present invention and it will be apparent to one skilled in the art that the present invention may be carried out using a large number of variations of the product, product components, methods steps set forth in the present description. As will be obvious to one of skill in the art, products and methods useful for the present invention can include a large number of optional composition and processing elements and steps.

As used herein, the singular forms “a”, “an”, and “the” include plural reference unless the context clearly dictates otherwise. Thus, for example, reference to “a cell” includes a plurality of such cells and equivalents thereof known to those skilled in the art. As well, the terms “a” (or “an”), “one or more” and “at least one” can be used interchangeably herein. It is also to be noted that the terms “comprising”, “including”, and “having” can be used interchangeably. The expression “of any of embodiments XX—YY” (wherein XX and YY refer to embodiment numbers) is intended to provide a multiple dependencies in the alternative form, and in some embodiments is interchangeable with the expression “as in any one of embodiments XX—YY.”

When a group of substituents is disclosed herein, it is understood that all individual members of that group and all subgroups, are disclosed separately. When a Markush group or other grouping is used herein, all individual members of the group and all combinations and subcombinations possible of the group are intended to be individually included in the disclosure. When a compound is described herein such that a particular isomer, enantiomer or diastereomer of the compound is not specified, for example, in a formula or in a chemical name, that description is intended to include each isomers and enantiomer of the compound described individual or in any combination.

Whenever a range is given in the specification, for example, a temperature range, a time range, or a composition or concentration range, all intermediate ranges and subranges, as well as all individual values included in the ranges given are intended to be included in the disclosure. It will be understood that any subranges or individual values in a range or subrange that are included in the description herein can be excluded from the embodiments herein.

All patents and publications mentioned in the specification are indicative of the levels of skill of those skilled in the art to which the invention pertains. References cited herein are incorporated by reference herein in their entirety to indicate the state of the art as of their publication or filing date and it is intended that this information can be employed herein, if needed, to exclude specific embodiments that are in the prior art.

As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the said embodiment. As used herein, “consisting essentially of” does not exclude materials or steps that do not materially affect the basic and novel characteristics of the embodiment. In each instance herein any of the terms “comprising”, “consisting essentially of” and “consisting of” may be replaced with either of the other two terms. The invention illustratively described herein suitably may be practiced in the absence of any element or elements, limitation or limitations which is not specifically disclosed herein.

One of ordinary skill in the art will appreciate that starting materials, biological materials, reagents, synthetic methods, purification methods, analytical methods, assay methods, and biological methods other than those specifically exemplified can be employed in the practice of the invention without resort to undue experimentation. All art-known functional equivalents, of any such materials and methods are intended to be included in this invention. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by embodiments herein.

Without wishing to be bound by any particular theory, there may be discussion herein of beliefs or understandings of underlying principles relating to the products and methods disclosed herein. It is recognized that regardless of the ultimate correctness of any mechanistic explanation or hypothesis, an embodiment of the invention can nonetheless be operative and useful.

In general, the terms and phrases used herein have their art-recognized meaning, which can be found by reference to standard texts, journal references and contexts known to those skilled in the art.

What is claimed is:

1. An additive, wherein the additive comprises a synthetic melanin nanoparticle, wherein the synthetic melanin nanoparticle comprises a synthetic melanin oligomer or a synthetic melanin polymer.

2. The additive of claim 1, wherein the synthetic melanin oligomer or synthetic melanin polymer comprises a plurality of covalently-bonded melanin base units.

3. The additive of claim 2, wherein the plurality of covalently-bonded melanin base units comprises a nitrogen-free precursor of allomelanin or an isomer thereof.

4. The additive of claim 2, wherein the plurality of covalently-bonded melanin base units comprises 3,4-dihydroxydopamine monomer units, 3,4-dioxydopamine monomer units, 3,4-dihydroxynaphthalene monomer units, 1,3-dihydroxynapthalene monomer units, 1,4-dihydroxynapthalene monomer units, 1,5-dihydroxynapthalene monomer units, 1,6-dihydroxynapthalene monomer units, 1,7-dihydroxynapthalene monomer units, 1,8-dihydroxynapthalene monomer units, 2,3-dihydroxynapthalene monomer units, 2,6-dihydroxynapthalene monomer units, 2,7-dihydroxynapthalene monomer units, I-3,4-dihydroxyphenylalanine monomer units, catechol units, or any combination thereof.

5. The additive of claim 2, wherein the plurality of covalently-bonded melanin base units comprises catechol units, 2,3-DHN monomer units, 2,6-DHN monomer units, 2.7-DHN monomer units, or any combination thereof.

6. The additive of claim 2, wherein the plurality of covalently-bonded melanin base units comprises about a 1:1 molar ratio of 1,8-dihydroxynapthalene: 2,3-dihydroxynapthalene monomer units.

7. The additive of claim 1, wherein the synthetic melanin oligomer or synthetic melanin polymer comprises a homopolymer or a copolymer.

8. The additive of claim 7, wherein the homopolymer comprises a 2,3 DHN homopolymer or a 2,6-DHN homopolymer.

9. The additive of claim 7, wherein the copolymer comprises two or more monomer units selected from the group consisting of: catechol, 2,7-DHN, 2,6-DHN, 2,3-DHN, and 1,8-DHN.

10. The additive of claim 7, wherein the copolymer comprises catechol and 2,6-DHN monomer units.

11. The additive of claim 10, wherein the copolymer comprises about 10-20 wt % of catechol monomer units and about 80-90 wt % of 2,6-DHN monomer units.

12. A pharmaceutical composition comprising the additive of claim 1 and a pharmaceutically acceptable excipient.

13. A method for protecting a cell-free reaction from damage, the method comprising contacting a cell-free reaction with the additive of claim 1.

14. A method for protecting a cell-free reaction from damage, the method comprising contacting a cell-free reaction with an additive to generate a cell-free reaction mixture, wherein the additive comprises a synthetic melanin nanoparticle, wherein the synthetic melanin nanoparticle comprises a synthetic melanin oligomer or a synthetic melanin polymer.

15. The method of claim 14, wherein the damage is at least partially caused by an exposure to solar light, ultraviolet (UV) irradiation, gamma irradiation, x-ray irradiation, or any combination thereof.

16. The method of claim 14, wherein the cell-free reaction comprises a cell-free peptide synthesis reaction, a cell-free metabolic pathway reaction, a cell-free biosensor reaction, a cell-free gene expression system, an E. coli-based cell-free system, or any combination thereof.

17. The method of claim 14, wherein the additive is present in the cell-free reaction at a concentration of between 0.25 mg/mL and 7.5 mg/mL, optionally between 0.25 mg/mL and 5 mg/mL, optionally between 0.25 mg/mL and 3 mg/mL, and/or optionally no greater than 3 mg/mL.

18. The method of claim 14, wherein the method further comprises freeze-drying the cell-free reaction after contacting the cell-free reaction with the additive.

19. The method of claim 14, wherein the synthetic melanin oligomer or polymer comprises a plurality of covalently-bonded melanin base units.

20. The method of claim 19, wherein the plurality of covalently-bonded melanin base units comprises 3,4-dihydroxydopamine monomer units, 3,4-dioxydopamine monomer units, 3,4-dihydroxynaphthalene monomer units, 1,3-dihydroxynapthalene monomer units, 1,4-dihydroxynapthalene monomer units, 1,5-dihydroxynapthalene monomer units, 1,6-dihydroxynapthalene monomer units, 1,7-dihydroxynapthalene monomer units, 1,8-dihydroxynapthalene monomer units, 2,3-dihydroxynapthalene monomer units, 2,6-dihydroxynapthalene monomer units, 2,7-dihydroxynapthalene monomer units, I-3,4-dihydroxyphenylalanine monomer units, catechol units, or any combination thereof.
